(19) United States
(12) Patent Application Publication
(10) Pub. No.: US 2026/0250675 A1
(43) Pub. Date: Aug. 27, 2026

(54) COMPOSITIONS AND METHODS FOR MODULATING C3

(71) Applicant: Leal Therapeutics, Inc., Worcester, MA (US)

(72) Inventors: Asa Abeliovich, New York, NY (US); Laura Heckman, Philadelphia, PA (US); Herve Rhinn, Paris (FR); Vinod Vathipadiekal, Stoneham, MA (US)

(73) Assignee: Leal Therapeutics, Inc., Worcester, MA (US)

(21) Appl. No.: 19/163,074

(22) PCT Filed: Mar. 8, 2024
(86) PCT No.: PCT/US2024/019104

§ 371 Date: Sep. 8, 2025

Related U.S. Application Data
(60) Provisional application No. 63/489,279, filed on Mar. 9, 2023.
(60) Provisional application No. 63/492,581, filed on Mar. 28, 2023.
(60) Provisional application No. 63/507,913, filed on Jun. 13, 2023.
(60) Provisional application No. 63/581,263, filed on Sep. 7, 2023.
(60) Provisional application No. 63/588,040, filed on Oct. 5, 2023.
(60) Provisional application No. 63/590,703, filed on Oct. 16, 2023.
(60) Provisional application No. 63/556,376, filed on Feb. 21, 2024.

(51) Int. Cl. C12N 15/113; A61K 31/712; A61K 31/7125; A61P 25/00; A61P 27/00; A61P 29/00; A61P 37/02

(52) U.S. Cl. CPC C12N 15/113; A61K 31/712; A61K 31/7125; A61P 25/00; A61P 27/00; A61P 29/00; A61P 37/02; C12N 2310/11; C12N 2310/315; C12N 2310/321; C12N 2310/322; C12N 2310/3231; C12N 2310/341; C12N 2310/346; C12N 2320/33

(57) ABSTRACT

Aspects of the disclosure relate to compositions and methods for modulating levels, transcription, splicing, and/or translation of one or more RNA transcripts (e.g., mRNA transcripts) in a cell or subject. The disclosure is based, in part, on isolated nucleic acids that bind to mRNA transcripts of genes involved in neuroinflammation, for example complement component C3 (C3). In some embodiments, compositions of the disclosure are useful for treating diseases or disorders associated with neuroinflammation, such as dry age-related macular degeneration (dry AMD) (e.g., dry AMD with geographic atrophy), multiple sclerosis (MS), peripheral neuropathies (e.g., Guillain Barr syndrome (GBS), Chronic inflammatory demyelinating polyneuropathy (CIDP), etc.), neuromyelitis optica (NO), myasthenia gravis (MG), Alzheimer's disease, frontotemporal dementia (FTD), iatrogenic neuroinflammation (e.g., inflammation associated with adeno-associated virus administration, ASO administration, antibody administration (such as Amyloid-Related Imaging Abnormalities (ARIA) neuroinflammation), etc.), and acute neuronal injury (e.g., traumatic brain injury (TBI), spinal cord injury, stroke, etc.).

### RELATED APPLICATIONS

This application is a national stage filing under 35 U.S.C. § 371 of international PCT application PCT/US2024/019104, filed Mar. 8, 2024, which claims priority under 35 U.S.C. § 119(e) to U.S. Provisional Application No. 63/489,279 filed on Mar. 9, 2023, U.S. Provisional Application No. 63/492,581 filed on Mar. 28, 2023, U.S. Provisional Application No. 63/507,913 filed on Jun. 13, 2023, U.S. Provisional Application No. 63/581,263 filed on Sep. 7, 2023, U.S. Provisional Application No. 63/588,040 filed on Oct. 5, 2023, U.S. Provisional Application No. 63/590,703 filed on Oct. 16, 2023, and U.S. Provisional Application No. 63/556,376 filed on Feb. 21, 2024, each of which is herein incorporated by reference in its entirety.

### REFERENCE TO AN ELECTRONIC SEQUENCE LISTING

The contents of the electronic sequence listing (L090770036US07-SEQ-KZM.xml; Size: 193,728 bytes; and Date of Creation: Sep. 8, 2025) are herein incorporated by reference in its entirety.

### BACKGROUND

The complement system is a part of the mammalian innate immune system that helps enable antibodies and phagocytic cells to kill microbes by promoting inflammation and attack on the cell membranes of pathogens. Complement C3 is a protein of the complement pathway that serves as a convergence point for each of the three complement activation pathways and regulator of terminal pathway.

### SUMMARY

Aspects of the disclosure relate to isolated nucleic acids that bind to mRNA transcripts of genes involved in neuroinflammation, for example complement component C3 (C3). In some embodiments, compositions of the disclosure are useful for treating diseases or disorders associated with neuroinflammation, such as dry age-related macular degeneration (dry AMD) (e.g., dry AMD with geographic atrophy), multiple sclerosis (MS), peripheral neuropathies (e.g., Guillain Barr syndrome (GBS), Chronic inflammatory demyelinating polyneuropathy (CIDP), etc.), neuromyelitis optica (NO), myasthenia gravis (MG), Alzheimer's disease (AD), frontotemporal dementia (FTD), iatrogenic neuroinflammation (e.g., inflammation associated with adeno-associated virus administration, ASO administration, antibody administration (such as Amyloid-Related Imaging Abnormalities (ARIA), for example, Amyloid-Related Imaging Abnormalities edema (ARIA-E) neuroinflammation), etc.), and acute neuronal injury (e.g., traumatic brain injury (TBI), spinal cord injury, stroke, etc.). In some embodiments, the diseases of disorders are associated with iatrogenic neuroinflammation. In some embodiments, the iatrogenic neuroinflammation is caused by a complement-mediated immune reaction resulting from administration of certain therapeutic agents or procedures, for example vaccines, tumor-necrosis-factor-alpha inhibitors (TNFAIs), immune-checkpoint inhibitors (ICIs), immunomodulators, certain viral vectors (e.g., AAV vectors, lentiviral vectors, etc.), ASOs, antibodies, or radiation therapy (e.g., cognitive deficits after irradiation for pediatric brain tumors). In some embodiments, the iatrogenic neuroinflammation comprises ARIA (e.g. ARIA-E). The disclosure is based, in part, on compositions and methods for modulating a level, transcription, splicing, and/or translation of one or more RNA transcripts (e.g., mRNA transcripts) in a cell or subject.

Accordingly, in some aspects, the disclosure provides an isolated nucleic acid that comprises a region of complementarity with a human C3 mRNA transcript, and a nucleotide sequence that is at least 60% identical (e.g., 60-70%, 70-80%, 80-90%, 90-95%, 95-99%, or 100% identical) to any one of the nucleotide sequences set forth in SEQ ID NOs: 1-210, and upon binding to the mRNA transcript decreases a level, transcription, splicing, and/or translation of functional C3 protein from the mRNA transcript.

In some embodiments, the isolated nucleic acid comprises RNA. In some embodiments, the isolated nucleic acid is an antisense oligonucleotide (ASO).

In some embodiments, the isolated nucleic acid comprises or consists of between 10 and 40 nucleotides. In some embodiments, the isolated nucleic acid comprises or consists of between 18 and 25 nucleotides.

In some embodiments, the isolated nucleic acid comprises one or more chemical modifications. In some embodiments, the one or more chemical modifications comprise one or more nucleoside modifications and/or one or more sugar-phosphate backbone modifications. In some embodiments, the one or more nucleoside modifications comprises a 2′-O-methyl (2′-OMe) modification, 2′-fluoro modification, or a locked nucleic acid (LNA) modification. In some embodiments, the one or more sugar-phosphate backbone modifications comprises a phosphorothioate backbone modification. In some embodiments, the isolated nucleic acid is fully chemically modified (e.g., contains a fully modified sugar-phosphate backbone, and all nucleotides of the isolated nucleic acid are chemically modified).

In some embodiments, the isolated nucleic acid comprises one or more deoxyribonucleotides. In some embodiments, the isolated nucleic acid is a gapmer.

In some embodiments, the region of complementarity is located in an untranslated region of the C3 mRNA transcript. In some embodiments, the untranslated region comprises a 5′ UTR, intron, or 3′ UTR of the C3 mRNA transcript.

In some embodiments, the region of complementarity is located in a protein coding region of the C3 mRNA transcript.

In some embodiments, the region of complementarity is located on an intron-exon boundary (e.g., the region of complementarity spans an intron exon boundary, such that the isolated nucleic acid hybridizes binds to both an intron and an exon at the same time) of the C3 mRNA transcript.

In some embodiments, the region of complementarity comprises at least 10, 11, 12, 13, 14, 15, 16, 17, 18, 19, or 20 continuous nucleotides of the sequence set forth in SEQ ID NO: 211.

In some embodiments, the nucleotide sequence comprises the nucleic acid sequence set forth in any one of the nucleotide sequences set forth in Table 1.

In some aspects, the disclosure provides a method for decreasing a level, transcription, splicing, and/or translation of Complement component C3 in a cell or subject, the method comprising administering an isolated nucleic acid as described herein to a subject in need thereof.

In some embodiments, the subject is characterized as having neuroinflammation.

In some embodiments, the subject comprises one or more mutations in a gene that is associated with neuroinflammation. In some embodiments, the gene is C3.

In some embodiments, the cell or subject is a human cell or subject.

In some embodiments, the subject has or is suspected of having a disease or disorder associated with neuroinflammation. In some embodiments, the disease or disorder is dry age-related macular degeneration (dry AMD) (e.g., dry AMD with geographic atrophy), multiple sclerosis (MS), peripheral neuropathies (e.g., Guillain Barr syndrome (GBS), Chronic inflammatory demyelinating polyneuropathy (CIDP), etc.), neuromyelitis optica (NO), myasthenia gravis (MG), Alzheimer's disease, frontotemporal dementia (FTD), iatrogenic neuroinflammation (e.g., inflammation associated with adeno-associated virus administration, ASO administration, antibody administration (such as Amyloid-Related Imaging Abnormalities (ARIA), for example, Amyloid-Related Imaging Abnormalities edema (ARIA-E) neuroinflammation), etc.), and acute neuronal injury (e.g., traumatic brain injury (TBI), spinal cord injury, stroke, etc.). In some embodiments, the neuroinflammation is iatrogenic neuroinflammation. In some embodiments, the iatrogenic neuroinflammation is a result of the subject being administered a therapy selected from a vaccine, tumor-necrosis-factor-alpha inhibitor (TNFAIs), immune-checkpoint inhibitor (ICI), immunomodulator, ASO, antibody or viral vector (e.g., AAV vector, lentiviral vector, etc.). In some embodiments, the iatrogenic neuroinflammation comprises ARIA (e.g., ARIA-E). In some embodiments, the subject has been administered radiation therapy. In some embodiments, the subject has been administered one or more antibodies for treatment of Alzheimer's disease, for example aducanumab. In some embodiments, the subject has a progranulin deficiency (e.g., comprises one or more mutations in a progranulin gene that reduces expression or activity of progranulin).

In some embodiments, the administration is systemic administration. In some embodiments, the systemic administration comprises intravenous injection.

In some embodiments, the administration comprises direct administration to a target tissue of the subject. In some embodiments, the direct administration comprises direct injection to the central nervous system (CNS) of the subject. In some embodiments, the direct administration comprises direct injection to the peripheral nervous system (PNS) of the subject.

In some embodiments, the direct administration comprises direct administration to the eye of the subject (e.g., via intraocular injection, topical injection, etc.).

In some embodiments, the administration comprises placing the subject in a Trendelenburg position during the administration.

In some aspects, the disclosure provides a method for decreasing serum C3 levels in a subject, the method comprising administering an isolated nucleic acid as described herein, to a subject in need thereof.

In some embodiments, the subject is characterized as having neuroinflammation. In some embodiments, the neuroinflammation in the subject is a result of the subject being administered a therapy selected from a vaccine, tumor-necrosis-factor-alpha inhibitor (TNFAIs), immune-checkpoint inhibitor (ICI), immunomodulator, or viral vector (e.g., AAV vector, lentiviral vector, etc.). In some embodiments, the subject has been administered radiation therapy. In some embodiments, the subject has been administered one or more antibodies for treatment of Alzheimer's disease. In some embodiments, the antibody is aducanumab.

In some embodiments, the subject comprises one or more mutations in a gene that is associated with neuroinflammation. In some embodiments, the gene is C3.

In some embodiments, the cell or subject is a human cell or subject.

In some embodiments, the subject has or is suspected of having a disease or disorder associated with neuroinflammation. In some embodiments, the disease or disorder is dry age-related macular degeneration (dry AMD) (e.g., dry AMD with geographic atrophy), multiple sclerosis (MS), peripheral neuropathies (e.g., Guillain Barr syndrome (GBS), Chronic inflammatory demyelinating polyneuropathy (CIDP), etc.), neuromyelitis optica (NO), myasthenia gravis (MG), Alzheimer's disease, frontotemporal dementia (FTD), iatrogenic neuroinflammation (e.g., inflammation associated with adeno-associated virus administration, ASO administration, antibody administration (such as Amyloid-Related Imaging Abnormalities (ARIA), for example, Amyloid-Related Imaging Abnormalities edema (ARIA-E) neuroinflammation), etc.), and acute neuronal injury (e.g., traumatic brain injury (TBI), spinal cord injury, stroke, etc.). In some embodiments, the subject has a progranulin deficiency (e.g., comprises one or more mutations in a progranulin gene that reduces expression or activity of progranulin).

In some embodiments, the administration is systemic administration. In some embodiments, the systemic administration comprises intravenous injection.

In some embodiments, the administration comprises direct administration to a target tissue of the subject. In some embodiments, the direct administration comprises direct injection to the central nervous system (CNS) of the subject. In some embodiments, the direct administration comprises direct injection to the peripheral nervous system (PNS) of the subject. In some embodiments, the direct administration comprises direct administration to the eye of the subject (e.g., via intraocular injection, topical injection, etc.).

In some embodiments, the administration comprises placing the subject in a Trendelenburg position during the administration.

In some aspects, the disclosure provides a method for preventing or treating a disease or disorder associated with neuroinflammation in a subject in need thereof, the method comprising administering to the subject an isolated nucleic acid as described herein.

In some embodiments, the subject is a human.

In some embodiments, the neuroinflammation is iatrogenic neuroinflammation. In some embodiments, the iatrogenic neuroinflammation comprises ARIA (e.g., ARIA-E). In some embodiments, the disease or disorder is dry age-related macular degeneration (dry AMD) (e.g., dry AMD with geographic atrophy), multiple sclerosis (MS), peripheral neuropathies (e.g., Guillain Barr syndrome (GBS), Chronic inflammatory demyelinating polyneuropathy (CIDP), etc.), neuromyelitis optica (NO), myasthenia gravis (MG), Alzheimer's disease, frontotemporal dementia (FTD), iatrogenic neuroinflammation (e.g., inflammation associated with adeno-associated virus administration, ASO administration, antibody administration (such as Amyloid-Related Imaging Abnormalities (ARIA), for example, Amyloid-Related Imaging Abnormalities edema (ARIA-E) neuroinflammation), etc.), and acute neuronal injury (e.g., traumatic brain injury (TBI), spinal cord injury, stroke, etc.).

In some embodiments, the administration comprises direct administration to a target tissue of the subject. In some embodiments, the direct administration comprises direct injection to the central nervous system (CNS) of the subject. In some embodiments, the direct administration comprises direct injection to the peripheral nervous system (PNS) of the subject. In some embodiments, the direct administration comprises direct administration to the eye of the subject (e.g., via intraocular injection, topical injection, etc.).

### BRIEF DESCRIPTION OF DRAWINGS

FIG. 1 shows a schematic depicting modulation of RNA (e.g., mRNA, such as mature mRNA or pre-mRNA) translation by antisense oligonucleotides (ASOs). Composition “A” represents an ASO that binds to the 5′ untranslated region (5′ UTR) of an RNA. Composition “B” represents an ASO that binds to an intron of an RNA. Composition “C” represents an ASO that binds to a splice boundary (e.g., a splice junction) between an exon and intron of an RNA. Composition “D” represents an ASO that binds to an exon (e.g., protein coding region) of an RNA. Composition “E” represents a combination of an ASO binding to a 3′ UTR of an RNA, alone or with a trans-regulator. Composition “F” represents a “gapmer” ASO that binds to an exon (e.g., protein coding region) of an RNA and mediates RNaseH decay. Composition “G” represents a “gapmer” ASO that binds to a 3′ UTR of an RNA, alone or with a trans-regulator, and mediates RNaseH decay. In some embodiments, ASOs binding to an RNA result in translation of a truncated protein that has a dominant negative effect on the wild-type, full-length protein.

FIGS. 2A-2C show representative data regarding expression profiling of human complement component 3 (C3). FIG. 2A shows bulk tissue gene expression of human C3; data indicate C3 mRNA is ubiquitously expressed. FIG. 2B shows a schematic depicting exons and introns present in the C3 gene. FIG. 2C shows representative data for exon expression analysis of human C3 splice variants in tissue.

FIG. 3 is a schematic depicting the primary AUG, and several exon-exon junctions of C3 mRNA transcript (SEQ ID NO: 212).

FIG. 4 shows representative data for in vitro reduction of C3 mRNA in Hep3B cells in a 2-dose screening assay.

FIG. 5 shows representative data for representative data for in vitro reduction of C3 mRNA in Hep3B cells. The top panel shows C3 mRNA levels presented relative to mock-transfected controls in function of the dose of transfected ASOs for the 16 tested C3 ASOs (Skippers and Gapmers shown; means are presented, error bars are Standard Error for N=2 biological replicates by group. The bottom panel shows maximum inhibition (log 2, Y-axis) plotted in function of the observed EC50 (X-axis); the most potent ASOs are in the lower left part of the dot-plot.

FIG. 6 shows representative data for representative data for in vitro reduction of C3 mRNA in Hep2G cells. The top panel shows C3 mRNA levels presented relative to mock-transfected controls in function of the dose of transfected ASOs for 15 tested C3 ASOs (Skippers in dark shading, Gapmers in light shading); means are presented, error bars are Standard Error for N=2 biological replicates by group The bottom panel shows maximum inhibition (log 2, Y-axis) plotted in function of the observed EC50 (X-axis); the most potent ASOs are in the lower left part of the dot-plot.

FIGS. 7A-7D show representative data for in vivo reduction of C3 mRNA levels in mouse brain. FIG. 7A shows relative C3 mRNA levels in hippocampus tissues of mouse subjects seven days after the last dose of a three-dose (e.g., 1 dose per week) series of ICV injections of vehicle (artificial CSF), 3 ug (total) of myriocin, a non-C3-specific ASO at a total dose of 300 ug (100 ug+100 ug+100 ug), C3 ASOs 1 and 2, each at a total dose of 300 ug (100 ug+100 ug+100 ug), or C3 ASO 3 at a total dose of 200 ug (100 ug+50 ug+50 ug). FIG. 7B shows relative C3 mRNA levels in cortex tissues of mouse subjects seven days after the last dose of a three-dose (e.g., 1 dose per week) series of ICV injections of vehicle (artificial CSF), 3 ug (total) of myriocin, a non-C3-specific ASO at a total dose of 300 ug (100 ug+100 ug+100 ug), C3 ASOs 1 and 2, each at a total dose of 300 ug (100 ug+100 ug+100 ug), or C3 ASO 3 at a total dose of 200 ug (100 ug+50 ug+50 ug). FIG. 7C shows relative C3 mRNA levels in hippocampus tissues of mouse subjects one week after the last of three ICV injections through a canula, with 1 week interval, of vehicle (artificial CSF), 100 ug+100 ug+100 ug of C3 ASO 1 or C3 ASO 2, or 100 ug+50 ug+50 ug of C3 ASO 3. FIG. 7D shows relative C3 mRNA levels in cortex tissues of mouse subjects one week after the last of three ICV injections through a canula, with 1 week interval, of vehicle (artificial CSF), 100 ug+100 ug+100 ug of C3 ASO 1 or C3 ASO 2, or 100 ug+50 ug+50 ug of ASO 3. “C3 ASO 1” comprises the nucleotide sequence of SEQ ID NO: 16, a gapmer structure, and the chemical modifications as set forth in Columns A and C of row 17 of Table 1. “C3 ASO 2” comprises the nucleotide sequence of SEQ ID NO: 156, a gapmer structure, and the chemical modifications as set forth in Columns A and C of row 157 of Table 1. “C3 ASO 3” comprises the nucleotide sequence of SEQ ID NO: 71, a gapmer structure, and the chemical modifications as set forth in Columns A and C of row 72 of Table 1.

FIGS. 8A-8L show representative lesion measurement data obtained following C3 ASO administration and subsequent laser-induced choroidal neovascularization in mouse subjects. Mouse subjects received intravitreal (IVT) injection of either phosphate buffered saline on day 0 (“Vehicle, 0 ug”), a 40 μg dose of Aflibercept on day 7 (“Aflibercept, 40 ug×1”), a single 50 μg dose of C3 ASO on day 0 (“C3 ASO, 50 ug×1”), or a 50 μg dose of C3 ASO on both day 0 and day 7 (“C3 ASO, 50 ug×2”). At day 14, subjects underwent laser-induced choroidal neovascularization. Fluorescein angiography and immunohistochemistry (IHC) imaging analyses were performed at day 21. Each bar indicates either the mean or median lesion area respective to the indicated group of subjects. Each dot represents an eye. N=8-16 eyes per group. Error bars indicated standard error of the mean. “C3 ASO” refers to an ASO comprising the nucleotide sequence of SEQ ID NO: 156, a gapmer structure, and the chemical modifications as set forth in Columns A and C of row 157 of Table 1 (alternatively referred to as “C3 ASO 2” herein). FIG. 8A shows the mean of the lesion area measurements corresponding to the indicated groups of subjects which were obtained from fluorescein angiography analyses. FIG. 8B shows the median of the lesion area measurements corresponding to the indicated groups of subjects which were obtained from fluorescein angiography analyses. FIG. 8C shows the mean of the lesion area measurements corresponding to the indicated groups of subjects which were obtained from fluorescein angiography analyses and compares effects of single IVT injection to repeated IVT injection of C3 ASO. FIG. 8D shows the median of the lesion area measurements corresponding to the indicated groups of subjects which were obtained from fluorescein angiography analyses and compares effects of single IVT injection to repeated IVT injection of C3 ASO. FIG. 8E shows the mean of the lesion area measurements corresponding to the indicated groups of subjects which were obtained from IHC analyses of flat mounts choroid/retinal pigment epithelium stained for isolectin. FIG. 8F shows the median of the lesion area measurements corresponding to the indicated groups of subjects which were obtained from IHC analyses of flat mounts choroid/retinal pigment epithelium stained for isolectin. (*): p<0.1 as determined by ANOVA and Dunnett's test analysis of lesion area measurements in treatment vs. vehicle groups. FIG. 8G shows the mean of the lesion area measurements corresponding to the indicated groups of subjects which were obtained from IHC analyses of flat mounts choroid/retinal pigment epithelium stained for isolectin and compares effects of C3 ASO single IVT injection to C3 ASO repeated IVT injection. The triangle above the graph represents the trend in mean lesion measurements across the indicated groups. *: p<0.05 as determined linear regression analysis of lesion area measurements. FIG. 8H shows the median of the lesion area measurements corresponding to the indicated groups of subjects which were obtained from IHC analyses of flat mounts choroid/retinal pigment epithelium stained for isolectin and compares effects of C3 ASO single IVT injection to C3 ASO repeated IVT injection. The triangle above the graph represents the trend in median lesion measurements across the indicated groups. For C3 ASO dose response, *: p<0.05 as determined linear regression analysis of lesion area measurements. Error bars indicate standard error of the mean. FIG. 8I shows C3 protein levels within the lesion area as determined by IHC analyses. For C3 ASO dose response, **p<0.01 by linear regression analysis. Error bars indicate standard error of the mean. FIG. 8J shows statistical analyses of the IHC data shown in FIG. 8I. Impact of aflibercept assessed by ANOVA with Dunnett's test comparing treatment groups vs. vehicle. FIG. 8K shows CD68 protein levels within the lesion area as determined by IHC analyses. For C3 ASO dose response, **p<0.01 by linear regression analysis. Error bars indicate standard error of the mean. FIG. 8L shows statistical analyses of the IHC data shown in FIG. 8K. Impact of aflibercept assessed by ANOVA with Dunnett's test comparing treatment groups vs. vehicle.

FIGS. 9A-9J show representative immunostimulatory effects of C3 ASO on human peripheral blood mononuclear cells (huPBMCs) that were harvested from healthy donors. huPBMCs were either untreated (“mock” and “media”), treated with a cytokine/chemokine response control agent (XD-01024, XD00366 transfection, poly(l:c) transfection, CL097, R837, TL8-506, ODN2395 transfection, ODN2395 gymnotic, ODN2216 transfection, ODN2216 gymnotic, ODN2006 transfection, or ODN2006 gymnotic), or treated with ASO at a concentration of 1 μM, 3 μM, or 10 μM for 24 hours (indicated on x-axes). Cytokine/chemokine levels were then analyzed using the MSD-U-Plex platform (indicated by y-axes). “C3 ASO 2” comprises the nucleotide sequence of SEQ ID NO: 156, a gapmer structure, and the chemical modifications as set forth in Columns A and C of row 157 of Table 1. Plots show mean+/−standard error. Each dot represents an individual donor. N=4 donors (2 male and 2 female). FIG. 9A shows analyses of IFN-a2a levels. FIG. 9B shows analyses of IFN-b levels. FIG. 9C shows analyses of IL-1B levels. FIG. 9D shows analyses of IL-6 levels. FIG. 9E shows analyses of IL-10 levels. FIG. 9F shows analyses of IP-10 levels. FIG. 9G shows analyses of MCP-1 levels. FIG. 9H shows analyses of MIP-1a levels. FIG. 9I shows analyses of MIP-1b levels. FIG. 9J shows analyses of TNF-α levels.

FIG. 10 shows a non-limiting example of a study design wherein non-human primate subjects were administered a series of four intrathecal injections of either vehicle (artificial CSF) or ASO at a dose of 80 mg (20 mg+20 mg+20 mg+20 mg). Each intrathecal injection was performed two weeks apart (days 0, 14, 28, and 42).

FIG. 11 shows ASO levels in dorsal root ganglion (DRG), hippocampus, lumbar spinal cord, motor cortex, prefrontal cortex, and temporal cortex samples obtained from injected non-human primate subjects and assessed by liquid chromatography-tandem mass spectrometry (LC-MS/MS). Non-human primate subjects received ASO at a dose of 80 mg (20 mg+20 mg+20 mg+20 mg) by intrathecal injection as illustrated in FIG. 10. The indicated samples were obtained at two weeks post-final injection of ASO (day 56). Each dot represents a sample from obtained from a different non-human primate subject. N=3 for each of the indicated groups of samples. Bars show mean+/−standard error of the mean. “C3 ASO 2” comprises the nucleotide sequence of SEQ ID NO: 156, a gapmer structure, and the chemical modifications as set forth in Columns A and C of row 157 of Table 1.

### DETAILED DESCRIPTION

Aspects of the disclosure relate to compositions and methods for modulating a level, transcription, splicing, and/or translation of one or more RNA transcripts (e.g., mRNA transcripts) in a cell or subject. The disclosure is based, in part, on isolated nucleic acids that bind to mRNA transcripts of genes involved in neuroinflammation, for example Complement component C3 (C3). In some embodiments, compositions of the disclosure are useful for treating diseases or disorders associated with neuroinflammation, such as dry age-related macular degeneration (dry AMD) (e.g., dry AMD with geographic atrophy), multiple sclerosis (MS), peripheral neuropathies (e.g., Guillain Barr syndrome (GBS), Chronic inflammatory demyelinating polyneuropathy (CIDP), etc.), neuromyelitis optica (NO), myasthenia gravis (MG), Alzheimer's disease, frontotemporal dementia (FTD), iatrogenic neuroinflammation (e.g., inflammation associated with adeno-associated virus administration, ASO administration, antibody administration (such as Amyloid-Related Imaging Abnormalities (ARIA), for example, Amyloid-Related Imaging Abnormalities edema (ARIA-E) neuroinflammation), etc.), and acute neuronal injury (e.g., traumatic brain injury (TBI), spinal cord injury, stroke, etc.). In some embodiments, the diseases of disorders are associated with iatrogenic neuroinflammation. In some embodiments, the iatrogenic neuroinflammation comprises ARIA (e.g., ARIA-E). In some embodiments, the iatrogenic neuroinflammation is caused by a complement-mediated immune reaction resulting from administration of certain therapeutic agents or procedures, for example vaccines, tumor-necrosis-factor-alpha inhibitors (TNFAIs), immune-checkpoint inhibitors (ICIs), immunomodulators, certain viral vectors (e.g., AAV vectors, lentiviral vectors, etc.), ASOs, antibodies, or radiation therapy. In some embodiments, the iatrogenic neuroinflammation comprises ARIA (e.g., ARIA-E).

Neuroinflammation Neuroinflammation generally refers to an innate immune system-driven inflammatory response that is centralized in the tissues of the central nervous system (CNS), for example brain and spinal cord tissue. However, in some embodiments, neuroinflammation encompasses inflammation affecting other tissues, for example peripheral nervous system tissue (PNS) and certain cells of the eye (e.g., inflammation of tissue innervating the eye, such as the optic nerve).

In some embodiments, the neuroinflammation is iatrogenic neuroinflammation. As used herein, “iatrogenic neuroinflammation” refers to neuroinflammation that is induced unintentionally by a physician or surgeon or by medical treatment or diagnostic procedures. Iatrogenic inflammation may be induced by administration of several different types of therapeutics to a subject or performance of certain therapeutic procedures on a subject, for example administration of vaccines, tumor-necrosis-factor-alpha inhibitors (TNFAIs), immune-checkpoint inhibitors (ICIs), immunomodulators, certain viral vectors (e.g., AAV vectors, lentiviral vectors, etc.), ASOs, or antibodies for example as described by Kelly et al., J Neuroimmunol. 2022 Sep. 15; 370:577928. doi: 10.1016/j.jneuroim.2022.577928. In some embodiments, administration of an AAV-based therapy to a subject induces iatrogenic neuroinflammation (e.g., as described by Smith et al. Front Immunol. 2022 Sep. 16; 13:999021. doi: 10.3389/fimmu.2022.999021). In some embodiments, iatrogenic neuroinflammation results from administration of an antibody therapeutic to a subject. For example, Alzheimer's disease subjects that have been administered aducanumab have been observed to exhibit neuroinflammation (e.g., Amyloid-Related Imaging Abnormalities (ARIA), for example, Amyloid-Related Imaging Abnormalities edema (ARIA-E) neuroinflammation), e.g., as described by Crehan et al. Alzheimer's Research & Therapy (2020) 12:12. In some embodiments, iatrogenic neuroinflammation in a subject results from radiation therapy.

Neuroinflammation may also occur as a result of endogenous host processes. For example, it has been described that subjects having progranulin deficiency exhibit neuroinflammation (e.g., as described by Lui et a. Cell. 2016 May 5; 165(4):921-35. doi: 10.1016/j.cell.2016.04.001). As used herein, a “progranulin deficiency” refers to aberrant expression or activity of progranulin in a subject. A progranulin deficiency may result from a subject having one or more mutations, insertions, or deletions in a progranulin (PGRN) gene, for example as described by Martens et al. J Clin Invest. 2012 November; 122(11):3955-9. Doi: 10.1172/JCI63113 and Yu et al. Arch Neurol. 2010 February; 67(2): 161-170.

Aspects of the disclosure relate to the inventors' recognition that certain nucleic acid molecules, for example isolated nucleic acids such as RNA processing modulators (e.g., antisense oligonucleotides), can be used to tune transcription, levels, splicing, and/or translation of certain mRNA encoded by genes associated with neuroinflammation (e.g., iatrogenic neuroinflammation such as Amyloid-Related Imaging Abnormalities (ARIA), for example, Amyloid-Related Imaging Abnormalities edema (ARIA-E) neuroinflammation).

A “gene associated with neuroinflammation” refers to a gene encoding a gene product (e.g., an mRNA, protein, etc.) that is genetically, biochemically, or functionally associated with a neuroinflammatory response (e.g., an increase in inflammation in cells or tissue of the central nervous system (CNS) or peripheral nervous system (PNS), including ocular cells or tissues) in a cell or subject. In some embodiments, a neuroinflammatory response comprises activation of the innate immune system in the brain, spinal cord, or ocular tissue of a subject.

The complement system, which is also known as complement cascade enhances the ability of antibodies and phagocytic cells to clear microbes and damaged cells from an organism, promote inflammation, and attack the pathogen's cell membrane. The complement system is activated by one of three pathways: classical, lectin, or alternative pathway.

Complement C3 is a protein of the complement pathway that is a convergence point for each of the three complement activation pathways (classical, lectin, and alternative), and functions as a regulator of the complement system terminal pathway. Upon innate immune system activation, C3 is cleaved into two substituent fragments, termed 3a and 3b. The C3a fragment is a 77 residue anaphylatoxin that binds to the C3a receptor (C3aR), and mediates a pro-inflammatory response, for example by causing mast cell degranulation of histamine. The C3b fragment is a protein that is involved in several biological processes, for example marking pathogens for opsonization, and providing a feedback signal for amplifying the innate immune response. Previous studies have shown that inhibition of Complement component C3 mediates protection from neuroinflammation.

Accordingly, in some embodiments, a gene associated with neuroinflammation encodes an mRNA encoding a Complement component C3 protein. In humans, C3 protein is encoded by the C3 gene, located on chromosome 19 (e.g., encoded by Ensembl ID NO: ENSG00000125730, Chromosome 19: 6,677,704-6,730,562 reverse strand). In some embodiments, C3 encodes a peptide that is represented by NCBI Reference Sequence NP_000055.2. In some embodiments, a C3 gene encodes an mRNA comprising the sequence set forth in NCBI Reference Sequence NM_000064.4. In some embodiments, an mRNA is encoded by a C3 gene and comprises the sequence set forth below:

NM_000064.4
(SEQ ID NO: 211)
ACTCCTCCCCATCCTCTCCCTCTGTCCCTCTGTCCCTCTGACCCTGCACTGTCCCAGCACCATGGGACCCACCTCAG
GTCCCAGCCTGCTGCTCCTGCTACTAACCCACCTCCCCCTGGCTCTGGGGAGTCCCATGTACTCTATCATCACCCCC
AACATCTTGCGGCTGGAGAGCGAGGAGACCATGGTGCTGGAGGCCCACGACGCGCAAGGGGATGTTCCAGTCACTGT
TACTGTCCACGACTTCCCAGGCAAAAAACTAGTGCTGTCCAGTGAGAAGACTGTGCTGACCCCTGCCACCAACCACA
TGGGCAACGTCACCTTCACGATCCCAGCCAACAGGGAGTTCAAGTCAGAAAAGGGGCGCAACAAGTTCGTGACCGTG
CAGGCCACCTTCGGGACCCAAGTGGTGGAGAAGGTGGTGCTGGTCAGCCTGCAGAGCGGGTACCTCTTCATCCAGAC
AGACAAGACCATCTACACCCCTGGCTCCACAGTTCTCTATCGGATCTTCACCGTCAACCACAAGCTGCTACCCGTGG
GCCGGACGGTCATGGTCAACATTGAGAACCCGGAAGGCATCCCGGTCAAGCAGGACTCCTTGTCTTCTCAGAACCAG
CTTGGCGTCTTGCCCTTGTCTTGGGACATTCCGGAACTCGTCAACATGGGCCAGTGGAAGATCCGAGCCTACTATGA
AAACTCACCACAGCAGGTCTTCTCCACTGAGTTTGAGGTGAAGGAGTACGTGCTGCCCAGTTTCGAGGTCATAGTGG
AGCCTACAGAGAAATTCTACTACATCTATAACGAGAAGGGCCTGGAGGTCACCATCACCGCCAGGTTCCTCTACGGG
AAGAAAGTGGAGGGAACTGCCTTTGTCATCTTCGGGATCCAGGATGGCGAACAGAGGATTTCCCTGCCTGAATCCCT
CAAGCGCATTCCGATTGAGGATGGCTCGGGGGAGGTTGTGCTGAGCCGGAAGGTACTGCTGGACGGGGTGCAGAACC
CCCGAGCAGAAGACCTGGTGGGGAAGTCTTTGTACGTGTCTGCCACCGTCATCTTGCACTCAGGCAGTGACATGGTG
CAGGCAGAGCGCAGCGGGATCCCCATCGTGACCTCTCCCTACCAGATCCACTTCACCAAGACACCCAAGTACTTCAA
ACCAGGAATGCCCTTTGACCTCATGGTGTTCGTGACGAACCCTGATGGCTCTCCAGCCTACCGAGTCCCCGTGGCAG
TCCAGGGCGAGGACACTGTGCAGTCTCTAACCCAGGGAGATGGCGTGGCCAAACTCAGCATCAACACACACCCCAGC
CAGAAGCCCTTGAGCATCACGGTGCGCACGAAGAAGCAGGAGCTCTCGGAGGCAGAGCAGGCTACCAGGACCATGCA
GGCTCTGCCCTACAGCACCGTGGGCAACTCCAACAATTACCTGCATCTCTCAGTGCTACGTACAGAGCTCAGACCCG
GGGAGACCCTCAACGTCAACTTCCTCCTGCGAATGGACCGCGCCCACGAGGCCAAGATCCGCTACTACACCTACCTG
ATCATGAACAAGGGCAGGCTGTTGAAGGCGGGACGCCAGGTGCGAGAGCCCGGCCAGGACCTGGTGGTGCTGCCCCT
GTCCATCACCACCGACTTCATCCCTTCCTTCCGCCTGGTGGCGTACTACACGCTGATCGGTGCCAGCGGCCAGAGGG
AGGTGGTGGCCGACTCCGTGTGGGTGGACGTCAAGGACTCCTGCGTGGGCTCGCTGGTGGTAAAAAGCGGCCAGTCA
GAAGACCGGCAGCCTGTACCTGGGCAGCAGATGACCCTGAAGATAGAGGGTGACCACGGGGCCCGGGTGGTACTGGT
GGCCGTGGACAAGGGCGTGTTCGTGCTGAATAAGAAGAACAAACTGACGCAGAGTAAGATCTGGGACGTGGTGGAGA
AGGCAGACATCGGCTGCACCCCGGGCAGTGGGAAGGATTACGCCGGTGTCTTCTCCGACGCAGGGCTGACCTTCACG
AGCAGCAGTGGCCAGCAGACCGCCCAGAGGGCAGAACTTCAGTGCCCGCAGCCAGCCGCCCGCCGACGCCGTTCCGT
GCAGCTCACGGAGAAGCGAATGGACAAAGTCGGCAAGTACCCCAAGGAGCTGCGCAAGTGCTGCGAGGACGGCATGC
GGGAGAACCCCATGAGGTTCTCGTGCCAGCGCCGGACCCGTTTCATCTCCCTGGGCGAGGCGTGCAAGAAGGTCTTC
CTGGACTGCTGCAACTACATCACAGAGCTGCGGCGGCAGCACGCGCGGGCCAGCCACCTGGGCCTGGCCAGGAGTAA
CCTGGATGAGGACATCATTGCAGAAGAGAACATCGTTTCCCGAAGTGAGTTCCCAGAGAGCTGGCTGTGGAACGTTG
AGGACTTGAAAGAGCCACCGAAAAATGGAATCTCTACGAAGCTCATGAATATATTTTTGAAAGACTCCATCACCACG
TGGGAGATTCTGGCTGTGAGCATGTCGGACAAGAAAGGGATCTGTGTGGCAGACCCCTTCGAGGTCACAGTAATGCA
GGACTTCTTCATCGACCTGCGGCTACCCTACTCTGTTGTTCGAAACGAGCAGGTGGAAATCCGAGCCGTTCTCTACA
ATTACCGGCAGAACCAAGAGCTCAAGGTGAGGGTGGAACTACTCCACAATCCAGCCTTCTGCAGCCTGGCCACCACC
AAGAGGCGTCACCAGCAGACCGTAACCATCCCCCCCAAGTCCTCGTTGTCCGTTCCATATGTCATCGTGCCGCTAAA
GACCGGCCTGCAGGAAGTGGAAGTCAAGGCTGCTGTCTACCATCATTTCATCAGTGACGGTGTCAGGAAGTCCCTGA
AGGTCGTGCCGGAAGGAATCAGAATGAACAAAACTGTGGCTGTTCGCACCCTGGATCCAGAACGCCTGGGCCGTGAA
GGAGTGCAGAAAGAGGACATCCCACCTGCAGACCTCAGTGACCAAGTCCCGGACACCGAGTCTGAGACCAGAATTCT
CCTGCAAGGGACCCCAGTGGCCCAGATGACAGAGGATGCCGTCGACGCGGAACGGCTGAAGCACCTCATTGTGACCC
CCTCGGGCTGCGGGGAACAGAACATGATCGGCATGACGCCCACGGTCATCGCTGTGCATTACCTGGATGAAACGGAG
CAGTGGGAGAAGTTCGGCCTAGAGAAGCGGCAGGGGGCCTTGGAGCTCATCAAGAAGGGGTACACCCAGCAGCTGGC
CTTCAGACAACCCAGCTCTGCCTTTGCGGCCTTCGTGAAACGGGCACCCAGCACCTGGCTGACCGCCTACGTGGTCA
AGGTCTTCTCTCTGGCTGTCAACCTCATCGCCATCGACTCCCAAGTCCTCTGCGGGGCTGTTAAATGGCTGATCCTG
GAGAAGCAGAAGCCCGACGGGGTCTTCCAGGAGGATGCGCCCGTGATACACCAAGAAATGATTGGTGGATTACGGAA
CAACAACGAGAAAGACATGGCCCTCACGGCCTTTGTTCTCATCTCGCTGCAGGAGGCTAAAGATATTTGCGAGGAGC
AGGTCAACAGCCTGCCAGGCAGCATCACTAAAGCAGGAGACTTCCTTGAAGCCAACTACATGAACCTACAGAGATCC
TACACTGTGGCCATTGCTGGCTATGCTCTGGCCCAGATGGGCAGGCTGAAGGGGCCTCTTCTTAACAAATTTCTGAC
CACAGCCAAAGATAAGAACCGCTGGGAGGACCCTGGTAAGCAGCTCTACAACGTGGAGGCCACATCCTATGCCCTCT
TGGCCCTACTGCAGCTAAAAGACTTTGACTTTGTGCCTCCCGTCGTGCGTTGGCTCAATGAACAGAGATACTACGGT
GGTGGCTATGGCTCTACCCAGGCCACCTTCATGGTGTTCCAAGCCTTGGCTCAATACCAAAAGGACGCCCCTGACCA
CCAGGAACTGAACCTTGATGTGTCCCTCCAACTGCCCAGCCGCAGCTCCAAGATCACCCACCGTATCCACTGGGAAT
CTGCCAGCCTCCTGCGATCAGAAGAGACCAAGGAAAATGAGGGTTTCACAGTCACAGCTGAAGGAAAAGGCCAAGGC
ACCTTGTCGGTGGTGACAATGTACCATGCTAAGGCCAAAGATCAACTCACCTGTAATAAATTCGACCTCAAGGTCAC
CATAAAACCAGCACCGGAAACAGAAAAGAGGCCTCAGGATGCCAAGAACACTATGATCCTTGAGATCTGTACCAGGT
ACCGGGGAGACCAGGATGCCACTATGTCTATATTGGACATATCCATGATGACTGGCTTTGCTCCAGACACAGATGAC
CTGAAGCAGCTGGCCAATGGTGTTGACAGATACATCTCCAAGTATGAGCTGGACAAAGCCTTCTCCGATAGGAACAC
CCTCATCATCTACCTGGACAAGGTCTCACACTCTGAGGATGACTGTCTAGCTTTCAAAGTTCACCAATACTTTAATG
TAGAGCTTATCCAGCCTGGAGCAGTCAAGGTCTACGCCTATTACAACCTGGAGGAAAGCTGTACCCGGTTCTACCAT
CCGGAAAAGGAGGATGGAAAGCTGAACAAGCTCTGCCGTGATGAACTGTGCCGCTGTGCTGAGGAGAATTGCTTCAT
ACAAAAGTCGGATGACAAGGTCACCCTGGAAGAACGGCTGGACAAGGCCTGTGAGCCAGGAGTGGACTATGTGTACA
AGACCCGACTGGTCAAGGTTCAGCTGTCCAATGACTTTGACGAGTACATCATGGCCATTGAGCAGACCATCAAGTCA
GGCTCGGATGAGGTGCAGGTTGGACAGCAGCGCACGTTCATCAGCCCCATCAAGTGCAGAGAAGCCCTGAAGCTGGA
GGAGAAGAAACACTACCTCATGTGGGGTCTCTCCTCCGATTTCTGGGGAGAGAAGCCCAACCTCAGCTACATCATCG
GGAAGGACACTTGGGTGGAGCACTGGCCCGAGGAGGACGAATGCCAAGACGAAGAGAACCAGAAACAATGCCAGGAC
CTCGGCGCCTTCACCGAGAGCATGGTTGTCTTTGGGTGCCCCAACTGACCACACCCCCATTCCCCCACTCCAGATAA
AGCTTCAGTTATATCTCACGTGTCTGGAGTTCTTTGCCAAGAGGGAGAGGCTGAAATCCCCAGCCGCCTCACCTGCA
GCTCAGCTCCATCCTACTTGAAACCTCACCTGTTCCCACCGCATTTTCTCCTGGCGTTCGCCTGCTAGTGTG

The skilled artisan recognizes that when referring to a gene sequence encoding an mRNA, the sequence of the mRNA is identical to the recited gene sequence, except that each instance of “T” is replaced with “U”.

In some embodiments, a C3 gene (or an mRNA encoded by a C3 gene) comprises one or more nucleotide substitutions, one or more nucleotide insertions, and/or one or more nucleotide deletions relative to a wild type C3 gene (or mRNA encoded by a wild type C3 gene), and may be referred to as a “mutant” C3 gene or a C3 variant. The number of nucleotide substitutions in a C3 variant may vary. In some embodiments, a C3 variant comprises between 1 and 20, 5 and 10, 2 and 15, 10 and 30, or 20 and 100 nucleotide substitutions, nucleotide insertions, and/or nucleotide deletions relative to a wild type C3 gene (or mRNA encoded by a wild type C3 gene). In some embodiments, the one or more nucleotide substitutions, one or more nucleotide insertions, and/or one or more nucleotide deletions results in an amino acid substitution in the protein encoded by the C3 variant. In some embodiments, the one or more nucleotide substitutions, one or more nucleotide insertions, and/or one or more nucleotide deletions results in a nonsense mutation (e.g., insertion of a premature stop codon) in an mRNA encoded by the C3 variant.

In some embodiments, the one or more nucleotide substitutions, one or more nucleotide insertions, and/or one or more nucleotide deletions results in a frameshift mutation of the C3 variant relative to a wild type C3 gene. In some embodiments, a mutation or mutations present in a C3 variant result in the production of one or more splice variants of C3 mRNA. A “splice variant” may refer to a mRNA resulting from one or more mutations in a DNA sequence that occur at the boundary of an exon and an intron (splice site) of a gene. Splice site mutations generally disrupt RNA splicing and result in the loss of exons or the inclusion of introns and an altered protein-coding sequence (e.g., a “splice variant”).

Aspects of the disclosure relate to isolated nucleic acids, for example RNA processing modulators (e.g., ASOs) that bind to one or more target regions of an mRNA encoded by a gene associated with neuroinflammation. In some embodiments, the isolated nucleic acids bind to more or more splice variants of a C3 gene (e.g., a human C3 splice variant). In some embodiments, an isolated nucleic acid described by the disclosure binds to a region of a C3 splice variant (e.g., mRNA encoded by a C3 variant) selected from an untranslated region (UTR). In some embodiments, the UTR is a 5′ UTR. In some embodiments, the UTR is a 3′ UTR. In some embodiments, the UTR is an intron. In some embodiments, an isolated nucleic acid described by the disclosure binds to an intron-exon boundary of a C3 splice variant (e.g., mRNA encoded by a C3 variant). An intron-exon boundary refers to a contiguous nucleotide sequence that includes portions of an intron and exon that are adjacent to one another in the mRNA transcript. In some embodiments, an isolated nucleic acid (e.g., antisense oligonucleotide) binds to an mRNA expressed from a particular allele of C3 (e.g., binds to a target mRNA in an allele-specific manner).

### Isolated Nucleic Acids

In some embodiments of the present disclosure, a nucleic acid is an isolated nucleic acid. In some cases, nucleic acids are alternatively referred to as oligonucleotides. In some embodiments, an isolated nucleic acid comprises DNA (e.g., deoxyribonucleotides). In some embodiments, an isolated nucleic acid comprises RNA (e.g., ribonucleotides). In some embodiments, an isolated nucleic acid comprises both DNA (e.g., deoxyribonucleotides) and RNA (e.g., ribonucleotides), such as an isolated nucleic acid comprising a gapmer structure that comprises a region of deoxyribonucleotides which are flanked by regions of ribonucleotides. An isolated nucleic acid may be single stranded or double stranded. In some embodiments, the isolated nucleic acid is an RNA oligonucleotide. In some embodiments, the isolated nucleic acid is a single stranded RNA oligonucleotide (which may also be referred to as a single stranded RNA polynucleotide).

As used herein, the term “isolated” means artificially produced. Artificial production of an isolated nucleic acid may be achieved, for example, through amplification in vitro through polymerase chain reaction (PCR), recombinant cloning, or chemical synthesis. Methods of synthesizing isolated nucleic acids, for example RNAs, are known in the art, for example as described by Soukchareun et al. Preparation and characterization of antisense oligonucleotide-peptide hybrids containing viral fusion peptides. Bioconjug Chem. 1995 January-February; 6(1):43-53. doi: 10.1021/bc00031a004. PMID: 7711103.

The length of an isolated nucleic acid may vary. In some embodiments, an isolated nucleic acid (e.g., a single stranded RNA) is 10, 15, 20, 25, 30, 40, 45, 50, 55, 60, 65, 70, 75, 80, 85, 90, 95, or up to 120 nucleotides in length. In some embodiments, an isolated nucleic acid ranges from about 1 to 100, 2 to 30, 5 to 20, 10 to 40, or 20 to 80 nucleotides in length. In some embodiments, an isolated nucleic acid is between 10 and 50 nucleotides in length. In some embodiments, an isolated nucleic acid comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, an isolated nucleic acid is more than 50 nucleotides in length (e.g., 60, 70, 80, 90, 100, etc., nucleotides in length). In some embodiments, an isolated nucleic acid is no greater than 200 nucleotides in length.

In some embodiments, an isolated nucleic acid of the disclosure comprises an antisense oligonucleotide comprising the sequence set forth in any one of SEQ ID NOs: 1-210 (provided in Column A of Table 1). In some embodiments, an isolated nucleic acid of the disclosure comprises an antisense oligonucleotide comprising at least 15 nucleotides (e.g., at least 15, at least 16, at least 17, at least 18, at least 19, or at least 20 nucleotides) of the any one of the sequences set forth in SEQ ID NOs: 1-210 (provided in column A of Table 1).

In some embodiments of the present invention, an isolated nucleic acid is modified (e.g., comprises one or more modifications, for example chemical modifications, such as those in Column C of Table 1). A modified nucleic acid may refer to an oligonucleotide that has been structurally altered in a non-natural manner (e.g., a manner that does not occur in nature). Nucleic acid modifications may be used to endow the nucleic acid with specific functional characteristics relative to unmodified nucleic acids. In some embodiments, modification of an isolated nucleic acid promotes binding of the isolated nucleic acid to a target molecule or increases stability of the isolated nucleic acid (e.g., makes the isolated nucleic acid resistant to enzymatic degradation).

In some embodiments, the one or more modifications is between 1 and 50 modifications, 2 and 20, 5 and 30, 10 and 40, or 15 and 50 modifications. In some embodiments, an isolated nucleic acid comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 modifications. In some embodiments, an isolated nucleic acid comprises more than 50 modifications (e.g., up to 60, 70, 80, 90, or 100, etc., modifications). In some embodiments, an isolated nucleic acid comprises chemical modifications on each nucleotide and each sugar-phosphate backbone linkage. Such a modified isolated nucleic acid may be referred to as a “fully modified” isolated nucleic acid. In some embodiments, not all nucleotides of an isolated nucleic acid are modified.

A chemical modification may comprise a modification of a nucleobase or a nucleotide, and/or a modification of a sugar-phosphate backbone (e.g., modification of one or more sugar-phosphate backbone linkages).

In some embodiments, an isolated nucleic acid of the disclosure comprises one or more chemical modification(s) listed in Column C of Table 1.

In some embodiments, an isolated nucleic acid comprises one or more modifications to a 5′ carbon atom (e.g., a 5′-carbon atom of a sugar) and/or one or more modifications to a 5-carbon of a nucleobase. Examples of modifications include, but are not limited to, 5-(2-amino)propyl uridine, 5-bromo uridine, 5-propyne uridine, 5-propenyl uridine, 5-carboxymethylaminomethyl-2-thiouracil, and 5-carboxymethylaminomethyl uracil. In other embodiments, the nucleic acid modification is targeted to the 6-carbon atom of a nucleobase. In some embodiments, an isolated nucleic acid comprises one or more modifications to a 6-carbon atom (e.g., a 6-carbon atom of a nucleobase) for example a 6-(2-amino)propyl uridine. In some embodiments, an isolated nucleic acid comprises one or more modifications to an 8-carbon atom (e.g., an 8-carbon atom of a nucleobase). Examples of 8 modifications include, but are not limited to, 8-bromo guanosine, 8-chloro guanosine, and 8-fluoroguanosine.

In some embodiments, an isolated nucleic acid comprises one or more modifications to a 2′ carbon of the sugar group. Examples of modified sugar groups include, but are not limited to, D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-2-O-methoxyethoxy, 2′-allyloxy (—OCH2CH═CH2), 2′-propargyl, 2′-propyl, ethynyl, ethenyl, propenyl, and cyano and the like. In some embodiments, a modified sugar moiety comprises a hexose and incorporated into an oligonucleotide as described (Augustyns, K., et al., Nucl. Acids. Res. 18:4711 (1992)). Other examples of 2′ modifications include, but are not limited to, substitutions of the bound OH group with H, OR, R, F, Cl, Br, I, SH, SR, NH, NHR, NR, COOR, or OR, wherein R is a substituted or unsubstituted aliphatic group. Other 2′ modifications are found in the art. The term “aliphatic,” as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties.

In some embodiments, an isolated nucleic acid modification a sugar-phosphate backbone modification. One example of a phosphate group modifications is substitution of an oxygen atom with a sulfur atom. In other embodiments, the backbone of the nucleic acid is modified. Examples of backbone modifications include, but are not limited to, phosphorothioate, borano-phosphate, alkyl phosphonate nucleic acid, peptide nucleic acid, and morpholino. Morpholino backbones are described, for example by Corey and Abrams Genome Biol. 2001; 2(5): reviews1015.1—reviews1015.3.

Other examples of modified bases include N4,N4-ethanocytosine, 7-deazaxanthosine, 7-deazaguanosine, 8-oxo-N6-methyladenine, 4-acetylcytosine, dihydrouracil, inosine, N6-isopentenyl-adenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 2-methylthio-N6-isopentenyladenine, pseudouracil,5-methyl-2-thiouracil, 2-thiouracil, 4-thiouracil, 2-thiocytosine, and 2,6-diaminopurine. Other examples of nucleic acid modifications are described for example by Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, Duffy. BMC Bio. 2020 Sep. 2(8):112, and U.S. Pat. No. 5,684,143.

Additional modifications of isolated nucleic acids (e.g., ASOs) are described by Duffy et al. BMC Biology volume 18, Article number: 112 (2020), the entire contents of which are incorporated herein by reference.

In some embodiments, an isolated nucleic acid of the disclosure comprises a nucleic acid sequence from Column A of Table 1 and one or more chemical modifications (or combinations of chemical modifications) from Column C of Table 1, optionally where Columns A and C are from the same row of Table 1.

### RNA Processing Modulators

Aspects of the disclosure relate to compositions (e.g., isolated nucleic acids, agents, etc.) that modulate mRNAs encoded by genes associated with neuroinflammation. In some embodiments, the gene associated with neuroinflammation is C3 (e.g., a human C3 gene). In some embodiments, a composition comprises an RNA processing modulator. As used herein, an “RNA processing modulator” or “RPM” refers to an agent that binds to, and up-regulates, down-regulates, or otherwise change function or activity, of a target mRNA (e.g., an mRNA encoded by a gene associated with neuroinflammation, such as C3, or a gene product, such as a protein encoded by the mRNA) by affecting transcription, levels, splicing, and/or translation of the mRNA. An RNA processing modulator may be an isolated nucleic acid or ASO as described herein. In some embodiments, an RNA processing modulator is an isolated nucleic acid that affects transcription, levels, splicing, and/or translation of a target mRNA (e.g., an mRNA encoded by a C3 gene). In some embodiments, an RNA processing modulator is an antisense oligonucleotide that affects transcription, levels, splicing, and/or translation of a target mRNA (e.g., an mRNA encoded by a C3 gene). In some embodiments, an mRNA (e.g., a target mRNA) is a pre-mRNA (e.g., an RNA that has been transcribed from a gene, such as a C3 gene, but has not been processed to remove introns, for example by splicing). In some embodiments, an mRNA is a mature mRNA that has been processed (e.g., an mRNA transcribed from a C3 gene and that has undergone processing).

In some embodiments, an RNA processing modulator upregulates transcription, levels, splicing, and/or translation of a target mRNA. Upregulation of transcription, levels, splicing, and/or translation may comprise binding to a regulatory region (e.g., an untranslated region, such as a 5′ UTR or 3′ UTR) of a target mRNA and reducing non-productive splicing or translation initiation from alternative start codons present in the target mRNA, for example through steric blocking of non-productive splice site(s) or alternative start codons (such as “upstream alternative start codons” located in the 5′ UTR of the target mRNA), or causing a mRNA frameshift (e.g., a splice variant) resulting in translation of a protein variant from the target mRNA that lacks one or more inhibitory domains.

The amount of upregulation of transcription, levels, splicing, and/or translation mediated by an RNA processing modulator may vary. In some embodiments, an RNA processing modulator increases transcription, levels, splicing, and/or translation of a target mRNA transcript (e.g., increases relative to a cell or subject prior to the administration of the RPM, or increases relative to a control cell or subject) between 1-fold and 100-fold, 2-fold and 10-fold, 5-fold and 20-fold, 10-fold and 30-fold, 20-fold and 50-fold, or 25-fold and 100-fold, or any value therebetween. In some embodiments, an RNA processing modulator increases transcription, levels, splicing, and/or translation of a target mRNA transcript more than 100-fold, for example at least 200-fold, 400-fold, 500-fold, or 1000-fold. In some embodiments, an RNA processing modulator increases transcription, levels, splicing, and/or translation of a target mRNA transcript no more than 1000-fold. In some embodiments, upregulation of a level, transcription, splicing, and/or translation of a target mRNA is useful to increase expression of a desired (e.g., wild-type) allele encoding the target mRNA.

In some embodiments, an RNA processing modulator downregulates transcription, levels, splicing, and/or translation of a target mRNA. Downregulation of transcription, levels, splicing, and/or translation may comprise binding to a regulatory region (e.g., an untranslated region, such as a 5′ UTR or 3′ UTR) of a target mRNA and blocking transcription the target mRNA, for example through steric blocking of a transcription initiation site, binding to an mRNA and subsequently initiating RNAse H-mediated degradation (e.g., in the context of a ‘gapmer’ RNA processing modulator), or causing an mRNA frameshift (e.g., a splice variant) resulting in translation of a protein variant from the target mRNA that is inactive, or has reduced function or activity (e.g., enzymatic activity, the ability to interact with other proteins to form protein complexes, etc.). In some embodiments, the resulting protein variant is a dominant negative protein variant. In some embodiments, downregulation of a level, transcription, splicing, and/or translation of a target mRNA is useful to increase expression of an undesirable (e.g., mutant, or disease-associated) allele encoding a target mRNA.

The amount of downregulation of transcription, levels, splicing, and/or translation mediated by an RNA processing modulator may vary. In some embodiments, an RNA processing modulator decreases transcription, levels, splicing, and/or translation of a target mRNA transcript between 1-fold and 100-fold, 2-fold and 10-fold, 5-fold and 20-fold, 10-fold and 30-fold, 20-fold and 50-fold, or 25-fold and 100-fold, or any value therebetween. In some embodiments, an RNA processing modulator decreases transcription, levels, splicing, and/or translation of a target mRNA transcript more than 100-fold, for example at least 200-fold, 400-fold, 500-fold, or 1000-fold. In some embodiments, an RNA processing modulator decreases transcription, levels, splicing, and/or translation of a target mRNA transcript no more than 1000-fold.

An RNA processing modulator may alter the number and/or character of splice variants of a target mRNA. In some embodiments, an RNA processing modulator increases (relative to natural transcription or translation of a target mRNA) the number of different splice variants of an mRNA, or the ratio between different splice variants of an mRNA. In some embodiments, an RNA processing modulator decreases (relative to natural transcription or translation of a target mRNA) the number of different splice variants of an mRNA, or the ratio between different splice variants of an mRNA. In some embodiments, contacting a target mRNA with an RNA processing modulator results in 1, 2, 3, 4, 5, 6, 7, 8, 9, 10, or more splice variants of the target mRNA being transcribed and/or translated. In some embodiments, contacting a target mRNA with an RNA processing modulator results in a single splice variant of the target mRNA being transcribed and/or translated.

The binding location of an RNA processing modulator may vary. In some embodiments, an RNA processing modulator affects splicing of the target mRNA. For example, an RNA processing modulator may bind to the target mRNA at a splice junction (e.g., a location spanning an intron-exon boundary) and mediate skipping of one or more exons in the mRNA transcript. In some embodiments, skipping of one or more exons in the target mRNA results in production of a truncated protein variant of the protein encoded by the target mRNA. In another example, an RNA processing modulator may bind to the target mRNA at a splice junction and mediate alternative splicing in which an intron is translated, and a protein variant of the target gene is produced. In some embodiments, an RNA processing modulator binds a target mRNA at a location comprising a coding sequence (e.g., a protein coding sequence or an exon).

In some embodiments, an RNA processing modulator comprises an agent selected from the group consisting of nucleic acid, peptide (including polypeptide), and small molecule. Examples of small molecule RNA processing inhibitors include but are not limited to translational readthrough-inducing drugs (TRIDs), such as certain aminoglycosides, non-aminoglycoside antibiotics (e.g., negamycin), ataluren (PTC124), and amlexanox. Examples of peptides include but are not limited to activator proteins (e.g., transcription factors), suppressor proteins (e.g., inducible cAMP early repressor (ICER), bZIP repressor, SP1 repressor, certain histone deacetylases, etc.), antibodies, etc. Examples of nucleic acids include but are not limited to suppressor tRNAs, dsRNA, siRNA, micro-RNA (miRNA), artificial miRNA (ami-RNA), aptamers, and antisense oligonucleotides. In some embodiments, an RNA processing modulator comprises an antisense oligonucleotide (ASO).

As used herein, the term, “antisense nucleic acid,” or “ASO” refers to a single stranded nucleic acid that has sequence complementarity to a target sequence and is specifically hybridizable, e.g., under stringent conditions, with a nucleic acid having the target sequence. An antisense nucleic acid is specifically hybridizable when binding of the antisense nucleic acid to the target nucleic acid is sufficient to produce complementary base pairing between the antisense nucleic acid and the target nucleic acid, and there is a sufficient degree of complementarity to reduce or avoid non-specific binding of the antisense nucleic acid to non-target nucleic acid under conditions in which specific binding is desired, e.g., under physiological conditions in the case of in vivo assays or therapeutic treatment, and in the case of in vitro assays, under conditions in which the assays are performed. In some embodiments, an ASO is chemically synthesized. An ASO may be a DNA polynucleotide, an RNA polynucleotide, or a DNA/RNA polynucleotide (e.g., an ASO comprising a gapmer structure that comprises a region of deoxyribonucleotides flanked by regions comprising ribonucleotides).

Complementary refers to the capacity for precise pairing between two nucleotides. For example, if a nucleotide at a certain position of an antisense nucleic acid is capable of hydrogen bonding with a nucleotide at the corresponding position of a target nucleic acid (e.g., target RNA), then the antisense nucleic acid and target nucleic acid are considered to be complementary to each other at that position. The antisense nucleic acid and target nucleic acid are complementary to each other when a sufficient number of corresponding positions in each molecule are occupied by nucleotides that can hydrogen bond with each other through their bases. Thus, “complementary” is a term that is used to indicate a sufficient degree of complementarity or precise pairing such that stable and specific binding occurs between the antisense nucleic acid and target nucleic acid. However, it should be appreciated that 100% complementarity is not required. For example, in some embodiments, an antisense nucleic acid (e.g., an oligonucleotide) may be at least 80% complementary to (e.g., at least 85%, 90%, 91%, 92%, 93%, 94%, 95%, 96%, 97%, 98%, 99% or 100% complementary to) the consecutive nucleotides of a target nucleic acid (e.g., a target nucleic acid comprising an mRNA sequence encoded by SEQ ID NO: 211).

Sequence identity, including determination of sequence complementarity for nucleic acid sequences, may be determined by sequence comparison and alignment algorithms known in the art. To determine the percent identity of two nucleic acid sequences, the sequences are aligned for optimal comparison purposes (e.g., gaps can be introduced in the first sequence or second sequence for optimal alignment). The nucleotides at corresponding nucleotide positions are then compared. When a position in the first sequence is occupied by the same residue as the corresponding position in the second sequence, then the molecules are identical at that position. In some embodiments, the percent identity between the two sequences is a function of the number of identical positions shared by the sequences (e.g., % homology=# of identical positions/total # of positions×100), optionally penalizing the score for the number of gaps introduced and/or length of gaps introduced.

In some embodiments, an antisense oligonucleotide has a length in a range of 5 to 40 nucleotides, 5 to 30 nucleotides, 10 to 30 nucleotides, 10 to 25 nucleotides, or 15 to 25 nucleotides. In some embodiments of the disclosure, an antisense oligonucleotide comprises a lengthof 5, 6, 7, 8, 9, 10, 11, 12, 13, 14, 15, 16, 17, 18, 19,20,21,22,23,24,25,26,27,28,29, 30, 31, 32, 33, 34, 35, 36, 37, 38, 39, or 40 nucleotides.

In some embodiments, an antisense nucleic acid comprises a region of complementarity that is perfectly complementary to a portion of a target nucleic acid (e.g., 100% of the nucleotides of the ASO hybridize to the nucleotides of the target RNA, such as a target mRNA (e.g., an mRNA sequence encoded by SEQ ID NO: 211)). However, it should be appreciated that in some embodiments, an antisense nucleic acid comprises less than 100% sequence complementarity with a target nucleic acid (e.g., 50%, 60%, 70%, 80%, 90%, 95%, or 99% of the nucleotides of the ASO hybridize to the nucleotides of the target RNA, such as a target mRNA (e.g., an mRNA sequence encoded by SEQ ID NO: 211)). In addition, to minimize the likelihood of off-target effects, an antisense nucleic acid may be designed to ensure that it does not have a sequence (e.g., of 5 or more consecutive nucleotides) that is complementary with an off-target nucleic acid (e.g., an mRNA that is not transcribed from a C3 gene).

In some embodiments, an antisense oligonucleotide comprises a region of complementarity with an mRNA encoded by (e.g., transcribed from) a C3 gene. In some embodiments, an antisense nucleic acid oligonucleotide comprises a region of complementarity with an mRNA encoded by the sequence as set forth in SEQ ID NO: 211. In some embodiments, the region of complementarity of the antisense nucleic acid hybridizes with at least 6, e.g., at least 7, at least 8, at least 9, at least 10, at least 15 or more consecutive nucleotides of a target nucleic acid (e.g., an mRNA encoded by the sequence set forth in SEQ ID NO: 211). In some embodiments, an antisense oligonucleotide comprises a region of complementarity with a 5′ UTR, 3′ UTR, an exonic sequence, a splice donor sequence, a splice acceptor sequence or a lariat branch point encoded by a human C3 gene. In some embodiments, an oligonucleotide binds to an mRNA expressed from a particular allele of C3 (e.g., binds to a target mRNA in an allele-specific manner).

In some embodiments, an antisense oligonucleotide comprises a region of complementarity with an mRNA encoded by (e.g., transcribed from) a C3 gene. In some embodiments, an antisense oligonucleotide comprises a region of complementarity with a pre-mRNA sequence encoded by a human C3 gene, for example (e.g., ENSG00000125730, Chromosome 19: 6,677,704-6,730,562 reverse strand). In some embodiments, the region of complementarity of the antisense nucleic acid hybridizes with at least 6, e.g., at least 7, at least 8, at least 9, at least 10, at least 15 or more consecutive nucleotides of a target nucleic acid (e.g., a pre-mRNA encoded by ENSG00000125730, Chromosome 19: 6,677,704-6,730,562 reverse strand). In some embodiments, the antisense oligonucleotide comprises a region of complementarity with at least 6, e.g., at least 7, at least 8, at least 9, at least 10, at least 15 or more consecutive nucleotides of an intron encoded by ENSG00000125730, Chromosome 19: 6,677,704-6,730,562 reverse strand. The skilled artisan recognizes that the forward strand of such a nucleic acid encoding a pre-mRNA transcript or mRNA transcript may also be targeted.

In some embodiments, an antisense oligonucleotide comprises a region of complementarity that is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 continuous nucleotides complementary with an mRNA encoded by the sequence as set forth in of SEQ ID NO: 211. In some embodiments, an antisense oligonucleotide comprising a region of complementarity with an mRNA transcript encoded by SEQ ID NO: 211 comprises at least 60% sequence identity (e.g., 60-70%, 70-80%, 80-90%, 90-95%, or more than 95% sequence identity) to a nucleic acid sequence set forth in any one of SEQ ID NOs: 1-210, as recited in Column A of Table 1. In some embodiments, an antisense oligonucleotide comprises a sequence of 10 or more contiguous nucleotides (e.g., 10, 11, 12, 13, 14, 15, 16, 17, 18, or more contiguous nucleotides) of any one of the sequences set forth in SEQ ID NOs: 1-210, as recited in Column A of Table 1. In some embodiments, an antisense oligonucleotide comprises the nucleic acid sequence set forth in any one of SEQ ID NOs: 1-210, as recited in Column A of Table 1. In some embodiments, an antisense oligonucleotide comprises a nucleotide sequence having one or more mismatches (e.g., one or more bases that is not complementary to the nucleotide at a given position of the target mRNA) relative to an mRNA transcript encoded by the sequence set forth in SEQ ID NO: 211. In some embodiments, an antisense oligonucleotide comprises a sequence having 1, 2, 3, 4, 5, 6, 7, 8, 9, or 10 mismatches relative to an mRNA transcript encoded by the sequence set forth in SEQ ID NO: 211. In some embodiments, an antisense oligonucleotide comprising one or more mismatches relative to an mRNA transcript encoded by SEQ ID NO: 211 comprises at least 60% sequence identity (e.g., 60-70%, 70-80%, 80-90%, 90-95%, or more than 95% sequence identity) to a sequence of 10 or more contiguous nucleotides of any one of the sequences set forth in SEQ ID NOs: 1-210, as recited in Column A of Table 1. In some embodiments, an antisense oligonucleotide comprising at least 60% sequence identity to a sequence of 10 or more contiguous nucleotides (e.g., 10, 11, 12, 13, 14, 15, 16, 17, 18, or more contiguous nucleotides) of any one of the sequences set forth in SEQ ID NOs: 1-210 differs at one or more nucleotide positions (e.g., 1, 2, 3, 4, 5, 6, 7, 8, 9, 10, or more nucleotide positions comprising a substitution, an insertion, or a deletion) relative to the sequence of 10 or more contiguous nucleotides of any one of the sequences set forth in SEQ ID NOs: 1-210, as recited in Column A of Table 1. In some embodiments, an antisense oligonucleotide comprising one or more mismatches relative to an mRNA transcript encoded by SEQ ID NO: 211 comprises at least 60% sequence identity (e.g., 60-70%, 70-80%, 80-90%, 90-95%, or more than 95% sequence identity) to a nucleic acid sequence set forth in any one of SEQ ID NOs: 1-210. In some embodiments, an antisense oligonucleotide comprising at least 60% sequence identity to a nucleic acid sequence set forth in any one of SEQ ID NOs: 1-210 differs at one or more nucleotide positions (e.g., 1, 2, 3, 4, 5, 6, 7, 8, 9, 10, or more nucleotide positions comprising a substitution, an insertion, or a deletion) relative to the nucleic acid sequence set forth in any one of SEQ ID NOs: 1-210.

In some embodiments, RNA processing modulators (e.g., antisense oligonucleotides) are provided in a homogeneous preparation, e.g., in which at least 85%, at least 90%, at least 95%, or at least 99% of the RNA processing modulators (e.g., antisense oligonucleotides) are identical. For example, in some embodiments, homogeneous preparations of antisense oligonucleotides are provided in which at least 85%, at least 90%, at least 95%, or at least 99% of the oligonucleotides in the preparation are 10 to 25 nucleotides in length and comprise a region of complementarity that is complementary with at least 6 contiguous nucleotides of an mRNA transcript encoded by a C3 gene (e.g., a C3 gene encoding an mRNA comprising the nucleic acid sequence set forth in SEQ ID NO: 211). In some embodiments, RNA processing modulators (e.g., antisense oligonucleotides) are provided in a heterogeneous preparation, e.g., comprising at least 2, 3, 4, 5, 6, 7, 8, 9, 10, or more different RNA processing modulators (e.g., antisense oligonucleotides each targeting a different sequence of a C3 mRNA transcript).

RNA processing modulators (e.g., antisense oligonucleotides) of the disclosure may be modified to achieve one or more desired properties, such as, for example, improved cellular uptake, improved stability, reduced immunogenicity, improved potency, improved target hybridization, susceptibility to RNAse cleavage, etc. In some embodiments, an antisense nucleic acid is modified such that when present in a cell that contains a C3 gene, it is capable of hybridizing with RNA transcribed from the C3 gene without inducing cleavage of the RNA by an RNase. In some embodiments, an antisense nucleic acid is modified such that when present in a cell that contains a C3 gene, it is capable of hybridizing with RNA transcribed from the C3 gene and inducing cleavage of the RNA by an RNase.

RNA processing modulators (e.g., antisense oligonucleotides, e.g. a nucleic acid sequence set forth in any one of SEQ ID NOs: 1-299, as recited in column A of Table 1) can be modified at a base moiety, sugar moiety and/or phosphate backbone. Accordingly, RNA processing modulators (e.g., antisense oligonucleotides) may have one or more modified nucleotides (e.g., a nucleotide analog) and/or one or more backbone modifications (e.g., a modified internucleotide linkage). RNA processing modulators (e.g., antisense oligonucleotides) may have a combination of modified and unmodified nucleotides. RNA processing modulators (e.g., antisense oligonucleotides) may also have a combination of modified and unmodified internucleotide linkages. RNA processing modulators (e.g., antisense oligonucleotides) may comprise one or more chemical modifications (or combinations of chemical modifications) from Column C of Table 1. In some embodiments, an RNA processing modulator comprises a nucleic acid sequence from Column A of Table 1 and one or more chemical modifications (or combinations of chemical modifications) from Column C of Table 1, where Columns A and C are from the same row of Table 1.

In some embodiments, the one or more modifications is between 1 and 50 modifications, 2 and 20, 5 and 30, 10 and 40, or 15 and 50 modifications. In some embodiments, an RNA processing modulator (e.g., antisense oligonucleotide) comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 modifications. In some embodiments, an RNA processing modulator (e.g., antisense oligonucleotide) comprises more than 50 modifications (e.g., 60, 70, 80, 90, 100, etc., modifications). In some embodiments, an RNA processing modulator (e.g., antisense oligonucleotide) comprises no more than 100 modifications. In some embodiments, an RNA processing modulator (e.g., antisense oligonucleotide) comprises chemical modifications on each nucleotide and each sugar-phosphate backbone linkage. Such a modified RNA processing modulator (e.g., antisense oligonucleotide) may be referred to as a “fully modified” RNA processing modulator (e.g., antisense oligonucleotide). In some embodiments, a fully modified antisense oligonucleotide comprises the nucleic acid sequence of any one of SEQ ID NOs: 1-210. In some embodiments, not all of the nucleotides of an antisense oligonucleotide are modified.

RNA processing modulators (e.g., antisense oligonucleotides) may include ribonucleotides, deoxyribonucleotides, and combinations thereof (e.g., RNA processing modulators comprising a gapmer structure). Examples of modified nucleotides which can be used in antisense nucleic acids include, for example, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, and 2,6-diaminopurine.

In some embodiments, a modified nucleotide is a 2′-modified nucleotide. For example, the 2′-modified nucleotide may be a 2′-deoxy, 2′-fluoro, 2′-O-methyl, 2′-O-methoxyethyl, 2′-amino and 2′-aminoalkoxy modified nucleotide. In some embodiments, the 2′-modified nucleotide comprises a 2′-O-4′-C methylene bridge, such as a locked nucleic acid (LNA) nucleotide. In some embodiments of a 2′ modified nucleotide the 2′-hydroxyl group is linked to the 3′ or 4′ carbon atom of the sugar ring thereby forming a bicyclic sugar moiety. In such embodiments, the linkage may be a methylene (—CH2-)ₙ group bridging the 2′ oxygen atom and the 3′ or 4′ carbon atom wherein n is 1 or 2. In some embodiments, a linkage comprises a cEt modification (e.g., a —CH3 replacing a hydrogen in the methylene group of the bridge).

RNA processing modulators (e.g., antisense oligonucleotides) may include combinations of LNA nucleotides and unmodified nucleotides. Antisense nucleic acids may include combinations LNA and RNA nucleotides. Antisense nucleic acids may include combinations LNA and DNA nucleotides. A further preferred oligonucleotide modification includes Locked Nucleic Acids (LNAs) in which the 2′-hydroxyl group is linked to the 3′ or 4′ carbon atom of the sugar ring thereby forming a bicyclic sugar moiety.

RNA processing modulators (e.g., antisense oligonucleotides) acids may also include nucleobase-modified nucleotides, e.g., nucleotides containing a non-naturally occurring nucleobase instead of a naturally occurring nucleobase. Bases may be modified to block the activity of adenosine deaminase, for example. Examples of modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

Within antisense nucleic acids (e.g., antisense oligonucleotides) of the disclosure, as few as one and as many as all nucleotides can be modified. In some embodiments, a modified RNA processing modulator (e.g., antisense oligonucleotide) will contain as few modified nucleotides as are necessary to achieve a desired level of in vivo stability and/or bioaccessibility or other desired property.

Certain antisense oligonucleotides may include non-ionic DNA analogs, such as alkyl- and aryl-phosphonates (in which the charged non-bridging oxygen is replaced by an alkyl or aryl group), phosphodiester and alkylphosphotriesters, in which the charged oxygen moiety is alkylated. Nucleic acids which contain a diol, such as tetraethyleneglycol or hexaethyleneglycol, at either or both termini have also been shown to be substantially resistant to nuclease degradation and may be used herein. In some embodiments, antisense nucleic acids may include at least one lipophilic substituted nucleotide analog and/or a pyrimidine-purine dinucleotide.

In some embodiments, RNA processing modulators (e.g., antisense oligonucleotides) may have one or two accessible 5′ ends. It is possible to create modified oligonucleotides having two such 5′ ends, for instance, by attaching two oligonucleotides through a 3′-3′ linkage to generate an oligonucleotide having one or two accessible 5′ ends. The 3′-3′-linkage may be a phosphodiester, phosphorothioate, or any other modified internucleoside bridge. Additionally, 3′-3′-linked oligonucleotides where the linkage between the 3′ terminal nucleosides is not a phosphodiester, phosphorothioate, or other modified bridge, can be prepared using an additional spacer, such as tri- or tetra-ethylenglycol phosphate moiety.

A phosphodiester internucleotide linkage of an RNA processing modulator (e.g., antisense oligonucleotide) can be replaced with a modified linkage. The modified linkage may be selected from, for example, phosphorothioate, phosphorodithioate, NR1R2-phosphoramidate, borano-phosphate, α-hydroxybenzyl phosphonate, phosphate-(C1-C21)-O-alkyl ester, phosphate-I[(C6-C12)aryl-(C1-C21)-O-alkyl]ester, (C1-C8)alkylphosphonate and/or (C6-C12)arylphosphonate bridges, and (C7-C12)-α-hydroxymethyl-aryl. In some embodiments, a triazole ring is used.

A phosphate backbone of the RNA processing modulators (e.g., antisense oligonucleotides) can be modified to generate peptide nucleic acid molecules. As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols, for example.

RNA processing modulators (e.g., antisense oligonucleotides) also be formulated as morpholino oligonucleotides. In such embodiments, the riboside moiety of each subunit of an oligonucleotide of the oligonucleotide reagent is converted to a morpholine moiety. Morpholinos may also be modified, e.g., as peptide conjugated morpholino, etc.

Aspects of the disclosure relate to RNA processing modulators (e.g., antisense oligonucleotides) comprising a “gapmer” structure. A “gapmer” refers to an antisense oligonucleotide comprising the following formula Xₙ₁—(Y)ₙ₂—(X)ₙ₃, where (X) is a ribonucleotide (e.g., an RNA base) and (Y) is a deoxyribonucleotide (e.g., DNA base), and where each of n1, n2, and n3 are an integer ranging from 1 to 50 (inclusive of all integers therebetween). In some embodiments, antisense oligonucleotides having a gapmer structure bind (e.g., hybridize) to a target mRNA (e.g., an mRNA encoded by a C3 gene) and induce ribonuclease H1 (RNAseH1)-mediated degradation of the target mRNA. Gapmer antisense oligonucleotides are known in the art, for example as described by Kasuya et al. Sci Rep. 2016; 6: 30377.

The number of DNA bases in a gapmer may vary. In some embodiments, a gapmer comprises between 1 and 10 DNA bases (e.g., 1, 2, 3, 4, 5, 6, 7, 8, 9, or 10 DNA bases). In some embodiments, a gapmer comprises between 2 and 6 DNA bases (e.g., 2, 3, 4, 5, or 6 DNA bases). The DNA bases of a gapmer antisense oligonucleotide may be positioned toward to 5′ end of the ASO (e.g., within 1, 2, 3, 4, 5, etc. nucleotides of the 5′ terminal nucleotide of the ASO), toward the 3′ end of the ASO (e.g., within 1, 2, 3, 4, 5, etc. nucleotides of the 3′ terminal nucleotide of the ASO), or in the middle of the ASO (e.g., having an equal number of RNA bases flanking the DNA bases).

In other embodiments, an RNA processing modulator (e.g., antisense oligonucleotide) can be linked to functional groups, such as peptides (e.g., for targeting host cell receptors in vivo), or agents facilitating transport across the cell membrane or the blood-brain barrier. For example, oligonucleotide reagents of the disclosure also may be modified with chemical moieties (e.g., cholesterol) that improve the in vivo pharmacological properties of the RNA processing modulator. In some embodiments, a functional group comprises a peptide, small molecule, sugar, lipid, nucleic acid, or combination of any of the foregoing.

Sequences and chemical modifications ofrepresentative RNA processing modulators (e.g., antisense oligonucleotides) targeting C3 (e.g., an mRNA encoded by a C3 gene, such as a pre-mRNA or mature mRNA) are shown in Columns A and C, respectively, of Table 1.

TABLE 1
Representative RPMs targeting C3

| Seq. | Column A | Column B | Column C |
|---|---|---|---|
| No. | Sequence (5′ to 3′) | Region | Chemistry |
| 1 | GACCAGGTAGGTGTAGTA | Exon12_Ext | Full PS; 2′MOE; 18 mer; 4-10-4 |
| 2 | TGAAGTGGATCTGGTAGG | Exon10_Ext | Full PS; 2′MOE; 18 mer |
| 3 | TGTCTGGATGAAGAGGTACC | | Full PS; 2′MOE; 20 mer; 5-10-5; PO after 2nd |
| | | | base from 5′ end, PO after 3rd base from 3′ end |
| 4 | TCAGTGGAGAAGACCTGCTG | Exon-6 | Full PS; 2′MOE; 20 mer |
| 5 | TAGAATTTCTCTGTAGGCTC | Exon | Full PS; 2′MOE; 20 mer; 5-10-5 |
| 6 | CCGGAATGTCCCAAGACAAG | Exon-5 | Full PS; 2′MOE; 20 mer |
| 7 | AGGTGGCCTGCACGGTCACG | Exon-3 | Full PS; 2′MOE; 20 mer |
| 8 | ACGTTGCCCATGTGGTTGGT | Exon-2 | Full PS; 2′MOE; 20 mer |
| 9 | CTGTCTGGATGAAGAGGTAC | | Full PS; 2′MOE; 20 mer; 5-10-5; PO after 2nd |
| | | | from 5′ end, PO after 3rd position from 3′ end |
| 10 | CCTGGCATTGTTTCTGGTTC | UTR3 | Full PS; 2′MOE; 20 mer; 5-10-5, PO after 2nd |
| | | | base from 5′ end, PO after 3rd base from 3′ end |
| 11 | TCAATGGCCATGATGTACTC | | Full PS; 2′MOE; 20 mer; 5-10-5, PO after 2nd |
| | | | base from 5′ end, PO after 3rd base from 3′ end |
| 12 | AGACCTGCTGTGGTGAGTTT | Exon-6 | Full PS; 2′MOE; 20 mer |
| 13 | GGTGAAGTGGATCTGGTA | Exon10_Ext | Full PS; 2′MOE; 18 mer; 4-10-4 |
| 14 | GAGAGAAGACCTTGACCA | Exon26_Ext | Full PS; 2′MOE; 18 mer |
| 15 | TCTGTCTGGATGAAGAGG | Exon-3 + Intron | Full PS; 2′MOE; 18 mer; 4-10-4 |
| 16 | TCTTGGTGAAGTGGATCT | | Full PS; 2′MOE; 18 mer; 4-10-4; PO after 2nd |
| | | | base from 5′ end, PO after 3rd base from 3′ end |
| 17 | TGGTGAGTTTTCATAGTAGG | Exon-6 | Full PS; 2′MOE; 20 mer |
| 18 | GACCAGGTAGGTGTAGTA | | Full PS; 2′MOE; 18 mer; 4-10-4, PO after 2nd |
| | | | base from 5′ end, PO after 3rd base from 3′ end |
| 19 | GGTGTAGATGGTCTTGTC | Exon-3 + Intron | Full PS; 2′MOE; 18 mer; 4-10-4 |
| 20 | GGCTCCACTATGACCTCGAA | Exon-7 | Full PS; 2′MOE; 20 mer |
| 21 | TCTCCTCGCTCTCCAGCCGC | Exon-2 | Full PS; 2′MOE; 20 mer |
| 22 | GCAGGAGAATTCTGGTCT | Exon23_Ext | Full PS; 2′MOE; 18 mer |
| 23 | CTGTCTGGATGAAGAGGT | | Full PS; 2′MOE; 18 mer; 4-10-4, PO after 2nd |
| | | | from 5′ end, PO after 3rd position from 3′ end |
| 24 | CCCTCACCTTTGGCTGTGGT | Exon28_Ext | Full PS; 2′MOE; 20 mer; 5-10-5, PO after 2nd |
| | | | base from 5′ end, PO after 3rd base from 3′ end |
| 25 | ATGGGACTCCCCAGAGCCAG | Exon-1 | Full PS; 2′MOE; 20 mer |
| 26 | GTACTCCTTCACCTCAAACT | Exon | Full PS; 2′MOE; 20 mer; 5-10-5 |
| 27 | GACCAGCTGTGAGTGTAGGC | Exon10_Ext | Full PS; 2′MOE; 20 mer; 5-10-5, PO after 2nd |
| | | | base from 5′ end, PO after 3rd base from 3′ end |
| 28 | CTCTGCAGGCTGACCAGCAC | Exon-3 | Full PS; 2′MOE; 20 mer |
| 29 | GTCTTGGTGAAGTGGATC | | Full PS; 2′MOE; 18 mer; 4-10-4, PO after 2nd |
| | | | from 5′ end, PO after 3rd position from 3′ end |
| 30 | TTTCTCTGTAGGCTCCACTA | Exon-7 | Full PS; 2′MOE; 20 mer |
| 31 | CATGAGGTCAAAGGGCAT | Exon10_Ext | Full PS; 2′MOE; 18 mer; 4-10-4 |
| 32 | GTCTTGTCTGTCTGGATGAA | Exon-3 | Full PS; 2′MOE; 20 mer |
| 33 | GTGGGTCCCATGGTGCTG | Exon- | Full PS; 2′MOE; 18 mer |
| | | 1 + primary | |
| | | AUG | |
| 34 | GTCTGTCTGGATGAAGAGGT | | Full PS; 2′MOE; 20 mer; 5-10-5; PO after 2nd |
| | | | from 5′ end, PO after 3rd position from 3′ end |
| 35 | GGTCAGCACAGTCTTCTCAC | Exon-2 | Full PS; 2′MOE; 20 mer |
| 36 | TATCTCTGTTCATTGAGCCA | | Full PS; 2′MOE; 20 mer; 5-10-5, PO after 2nd |
| | | | base from 5′ end, PO after 3rd base from 3′ end |
| 37 | AGCACCATGGTCTCCTCGCT | Exon-2 | Full PS; 2′MOE; 20 mer |
| 38 | GGTAGATGATGAGGGTGT | Exon35_Ext | Full PS; 2′MOE; 18 mer; 4-10-4 |
| 39 | CTGGCCCATGCTGTGAGGAG | Exon-6 + Intron | Full PS; 2′MOE; 20 mer |
| 40 | GTCTGGATGAAGAGGTAC | | Full PS; 2′MOE; 18 mer; 4-10-4, PO after 2nd |
| | | | from 5′ end, PO after 3rd position from 3′ end |
| 41 | CTGGATGAAGAGGTACCC | Exon-3 + Intron | Full PS; 2′MOE; 18 mer |
| 42 | TGTCTGGATGAAGAGGTA | | Full PS; 2′MOE; 18 mer; 4-10-4, PO after 2nd |
| | | | from 5′ end, PO after 3rd position from 3′ end |
| 43 | CTTCACCTCAAACTCAGT | Exon-6 + Intron | Full PS; 2′MOE; 18 mer |
| 44 | CCTGACATACTTGACGAGTT | Intron + Exon-5 | Full PS; 2′MOE; 20 mer |
| 45 | GTAGATGATGAGGGTGTT | Exon35_Ext | Full PS; 2′MOE; 18 mer |
| 46 | TGTCTGTCTGGATGAAGAGG | Exon-3 + Intron | Full PS; 2′MOE; 20 mer; 5-10-5, PO after 2nd |
| | | | base from 5′ end, PO after 3rd base from 3′ end |
| 47 | AGGTAGATGATGAGGGTGTT | Exon35_Ext | Full PS; 2′MOE; 20 mer; 5-10-5, PO after 2nd |
| | | | base from 5′ end, PO after 3rd base from 3′ end |
| 48 | TCTGTAGGTTCATGTAGTTG | Exon | Full PS; 2′MOE; 20 mer; 5-10-5 |
| 49 | TAGATGGTCTTGTCTGTC | Exon-3 + Intron | Full PS; 2′MOE; 18 mer; 4-10-4 |
| 50 | GAGCTGGGTTGTCTGAAGGC | Exon25_Ext | Full PS; 2′MOE; 20 mer; 5-10-5, PO after 2nd |
| | | | base from 5′ end, PO after 3rd base from 3′ end |
| 51 | GGTCTTGTCTGTCTGGAT | Exon-3 + Intron | Full PS; 2′MOE; 18 mer |
| 52 | GGCAAGACGCCAAGCTGGTT | Exon-5 | Full PS; 2′MOE; 20 mer |
| 53 | GTTCTGAGAAGACAAGGAGT | Exon | Full PS; 2′MOE; 20 mer; 5-10-5 |
| 54 | TCATGTAGTTGGCTTCAA | Exon28_Ext | Full PS; 2′MOE; 18 mer |
| 55 | CACCTCAAACTCAGTGGAGA | Exon-6 | Full PS; 2′MOE; 20 mer |
| 56 | TGACGAGTTCCGGAATGTCC | Exon | Full PS; 2′MOE; 20 mer; 5-10-5 |
| 57 | AGTCCCTCACCTGGCGGTGA | Intron + Exon-7 | Full PS; 2′MOE; 20 mer |
| 58 | CTTACCGTACTCCTTCACCT | Intron + Exon-6 | Full PS; 2′MOE; 20 mer |
| 59 | AGAGAGAAGACCTTGACCAC | Exon26_Ext | Full PS; 2′MOE; 20 mer; 5-10-5, PO after 2nd |
| | | | base from 5′ end, PO after 3rd base from 3′ end |
| 60 | TTGTCTGTCTGGATGAAG | Exon-3 + Intron | Full PS; 2′MOE; 18 mer |
| 61 | GCATTGTTTCTGGTTCTCTT | | Full PS; 2′MOE; 20 mer; 5-10-5, PO after 2nd |
| | | | base from 5′ end, PO after 3rd base from 3′ end |
| 62 | GCTCAATGGCCATGATGTAC | Exon39_Ext | Full PS; 2′MOE; 20 mer; 5-10-5, PO after 2nd |
| | | | base from 5′ end, PO after 3rd base from 3′ end |
| 63 | TAGATGATGAGGGTGTTC | Exon35_Ext | Full PS; 2′MOE; 18 mer; 4-10-4 |
| 64 | GTCTTGTCTGTCTGGATG | Exon-3 + Intron | Full PS; 2′MOE; 18 mer; 4-10-4 |
| 65 | TTGACGGTGAAGATCCGATA | Exon-4 | Full PS; 2′MOE; 20 mer |
| 66 | GAGCTTGTTCAGCTTTCCAT | Exon | Full PS; 2′MOE; 20 mer; 5-10-5 |
| 67 | GCTTGGCTGGGTGACTGTAC | Exon30_Ext | Full PS; 2′MOE; 20 mer; 5-10-5, PO after 2nd |
| | | | base from 5′ end, PO after 3rd base from 3′ end |
| 68 | ACGAACTTGTTGCGCCCCTT | Exon-3 | Full PS; 2′MOE; 20 mer |
| 69 | GTGTAGATGGTCTTGTCT | Exon-3 + Intron | Full PS; 2′MOE; 18 mer |
| 70 | TTGGTGAAGTGGATCTGGTA | | Full PS; 2′MOE; 20 mer; 5-10-5; PO after 2nd |
| | | | from 5′ end, PO after 3rd position from 3′ end |
| 71 | GTCTTGGTGAAGTGGATCTG | | Full PS; 2′MOE; 20 mer; 5-10-5; PO after 2nd |
| | | | from 5′ end, PO after 3rd position from 3′ end |
| 72 | CCATGAGGTCAAAGGGCA | Exon10_Ext | Full PS; 2′MOE; 18 mer |
| 73 | TGGGCAGTTGGAGGGACACA | Exon30_Ext | Full PS; 2′MOE; 20 mer; 5-10-5, PO after 2nd |
| | | | base from 5′ end, PO after 3rd base from 3′ end |
| 74 | CTTGTCTGTCTGGATGAA | Exon-3 + Intron | Full PS; 2′MOE; 18 mer; 4-10-4 |
| 75 | CTCCAGCCGCAAGATGTTGG | Exon-2 | Full PS; 2′MOE; 20 mer |
| 76 | TTGGTGAAGTGGATCTGG | Exon10_Ext | Full PS; 2′MOE; 18 mer; 4-10-4 |
| 77 | TCTGGATGAAGAGGTACC | Exon-3 + Intron | Full PS; 2′MOE; 18 mer; 4-10-4 |
| 78 | CAGGCCCTTCTCGTTATAGA | Exon-7 | Full PS; 2′MOE; 20 mer |
| 79 | CAAGGAGTCCTGCTTGACCG | Exon | Full PS; 2′MOE; 20 mer; 5-10-5 |
| 80 | AAGTCTCCTGCTTTAGTGAT | Exon | Full PS; 2′MOE; 20 mer; 5-10-5 |
| 81 | CGTGAAGGTGACGTTGCCCA | Exon-2 | Full PS; 2′MOE; 20 mer |
| 82 | ACAGTGTAGGATCTCTGTAG | | Full PS; 2′MOE; 20 mer; 5-10-5; PO after 2nd |
| | | | base from 5′ end, PO after 3rd base from 3′ end |
| 83 | TGTCTGTCTGGATGAAGA | Exon-3 + Intron | Full PS; 2′MOE; 18 mer; 4-10-4 |
| 84 | ACAGTGTAGGATCTCTGTAG | Exon | Full PS; 2′MOE; 20 mer; 5-10-5 |
| 85 | TCCACTATGACCTCGAAACT | Exon | Full PS; 2′MOE; 20 mer; 5-10-5 |
| 86 | TAGAATTTCTCTGTAGGCTC | | Full PS; 2′MOE; 20 mer; 5-10-5, PO after 2nd |
| | | | base from 5′ end, PO after 3rd base from 3′ end |
| 87 | TATCTCTGTTCATTGAGCCA | Exon | Full PS; 2′MOE; 20 mer; 5-10-5 |
| 88 | CTCAATGTTGACCATGACCG | Exon-4 | Full PS; 2′MOE; 20 mer |
| 89 | TCGTTATAGATGTAGTAGAA | Exon-7 | Full PS; 2′MOE; 20 mer |
| 90 | GGTCACCCTCTATCTTCA | Exon14_Ext | Full PS; 2′MOE; 18 mer |
| 91 | TGGCTGGCACCTCAATGTTG | Intron + Exon-4 | Full PS; 2′MOE; 20 mer |
| 92 | CGTACTCCTTCACCTCAAAC | Exon-6 | Full PS; 2′MOE; 20 mer |
| 93 | TGTCTGGATGAAGAGGTACC | | Full PS; 2′MOE; 20 mer; 5-10-5, PO after 2nd |
| | | | base from 5′ end, PO after 3rd base from 3′ end |
| 94 | TGGTCTTGTCTGTCTGGA | Exon-3 + Intron | Full PS; 2′MOE; 18 mer; 4-10-4 |
| 95 | GTCTGGATGAAGAGGTAC | Exon-3 + Intron | Full PS; 2′MOE; 18 mer |
| 96 | GAGAGAAGACCTTGACCA | Exon26_Ext | Full PS; 2′MOE; 18 mer; 4-10-4 |
| 97 | TGTAGATGGTCTTGTCTG | Exon-3 + Intron | Full PS; 2′MOE; 18 mer; 4-10-4 |
| 98 | GGGTGTAGATGGTCTTGT | Exon-3 + Intron | Full PS; 2′MOE; 18 mer |
| 99 | TGACCAGCACCACCTTCTCC | Exon-3 | Full PS; 2′MOE; 20 mer |
| 100 | CTCCTCTTACCGTACTCCTT | Intron + Exon-6 | Full PS; 2′MOE; 20 mer |
| 101 | TCTGTCTGGATGAAGAGG | | Full PS; 2′MOE; 18 mer; 4-10-4, PO after 2nd |
| | | | from 5′ end, PO after 3rd position from 3′ end |
| 102 | ATGGTCTTGTCTGTCTGG | Exon-3 + Intron | Full PS; 2′MOE; 18 mer |
| 103 | TGTAGGATCTCTGTAGGT | Exon28_Ext | Full PS; 2′MOE; 18 mer |
| 104 | AAGATCCGATAGAGAACTGG | Exon-4 + Intron | Full PS; 2′MOE; 20 mer |
| 105 | TTGACGAGTTCCGGAATGTC | Exon-5 | Full PS; 2′MOE; 20 mer |
| 106 | TGACCTCGAAACTGGGCAGC | Exon-7 | Full PS; 2′MOE; 20 mer |
| 107 | GACCAGCTGTGAGTGTAG | Exon10_Ext | Full PS; 2′MOE; 18 mer |
| 108 | GGCTGGATTGTGGAGTAG | Exon21_Ext | Full PS; 2′MOE; 18 mer |
| 109 | CAGCTTGTGGTTGACGGTGA | Exon-4 | Full PS; 2′MOE; 20 mer |
| 110 | CTCACCTGGCGGTGATGGTG | Intron + Exon-7 | Full PS; 2′MOE; 20 mer |
| 111 | TATTGAGCCAAGGCTTGGAA | Exon | Full PS; 2′MOE; 20 mer; 5-10-5 |
| 112 | GGACCAGGTAGGTGTAGT | Exon12_Ext | Full PS; 2′MOE; 18 mer |
| 113 | TCTTGTCTGTCTGGATGA | Exon-3 + Intron | Full PS; 2′MOE; 18 mer |
| 114 | GGTGTAGATGGTCTTGTCTG | Exon-3 | Full PS; 2′MOE; 20 mer |
| 115 | TTGAACTCCCTGTTGGCTGG | Exon-3 | Full PS; 2′MOE; 20 mer |
| 116 | ACCATGACCGTCCGGCCCAC | Exon-4 | Full PS; 2′MOE; 20 mer |
| 117 | GAGGAGTGAGCAGCGC | 5'UTR + Intron | Full PS; 2′MOE; 16 mer |
| 118 | CAAGCTGGTTCTGAGAAGAC | Exon-5 | Full PS; 2′MOE; 20 mer |
| 119 | CCACGGGTAGCAGCTTGT | Exon-4 | Full PS; 2′MOE; 18 mer |
| 120 | GGCACCTCAATGTTGACCAT | Intron + Exon-4 | Full PS; 2′MOE; 20 mer |
| 121 | GGCTCTATGAGAAAGAGG | Exon28_Ext | Full PS; 2′MOE; 18 mer |
| 122 | TGTCTGGATGAAGAGGTACC | Exon | Full PS; 2′MOE; 20 mer; 5-10-5 |
| 123 | GATAGAGTACCTGTCGGAGT | Exon-2 + Intron | Full PS; 2′MOE; 20 mer |
| 124 | CAGCTGTGACTGTGAAACCC | Exon31_Ext | Full PS; 2′MOE; 20 mer; 5-10-5, PO after 2nd |
| | | | base from 5′ end, PO after 3rd base from 3′ end |
| 125 | GTGATGATAGAGTACCTGTC | Exon-2 + Intron | Full PS; 2′MOE; 20 mer |
| 126 | GTTGGCTGGGATCTAGGCGT | Exon-3 + Intron | Full PS; 2′MOE; 20 mer |
| 127 | TCTGGATGAAGAGGTACC | | Full PS; 2′MOE; 18 mer; 4-10-4, PO after 2nd |
| | | | from 5′ end, PO after 3rd position from 3′ end |
| 128 | GCTTGACCGGGATGCCTTCC | Exon-5 | Full PS; 2′MOE; 20 mer |
| 129 | GAAGATGACAAAGGCAGTTC | Exon | Full PS; 2′MOE; 20 mer; 5-10-5 |
| 130 | GTCTGTCTGGATGAAGAG | | Full PS; 2′MOE; 18 mer; 4-10-4, PO after 2nd |
| | | | from 5′ end, PO after 3rd position from 3′ end |
| 131 | CCAAGACAAGGGCAAGACG | Exon-5 | Full PS; 2′MOE; 20 mer |
| | C | | |
| 132 | AGGTAGATGATGAGGGTG | Exon35_Ext | Full PS; 2′MOE; 18 mer |
| 133 | TCAATGGCCATGATGTACTC | Exon | Full PS; 2′MOE; 20 mer; 5-10-5 |
| 134 | CTGCACTCACCGTGAAGGTG | Intron + Exon-2 | Full PS; 2′MOE; 20 mer |
| 135 | GTAGATGGTCTTGTCTGT | Exon-3 + Intron | Full PS; 2′MOE; 18 mer |
| 136 | GTCTGTCTGGATGAAGAG | Exon-3 + Intron | Full PS; 2′MOE; 18 mer |
| 137 | GTCTTGGTGAAGTGGATC | Exon10_Ext | Full PS; 2′MOE; 18 mer |
| 138 | CTTGGTGAAGTGGATCTGGT | Exon10_Ext | Full PS; 2′MOE; 20 mer; 5-10-5, PO after 2nd |
| | | | base from 5′ end, PO after 3rd base from 3′ end |
| 139 | TCTGTAGGTTCATGTAGTTG | | Full PS; 2′MOE; 20 mer; 5-10-5, PO after 2nd |
| | | | base from 5′ end, PO after 3rd base from 3′ end |
| 140 | AGGCACAAAGTCAAAGTCTT | Exon | Full PS; 2′MOE; 20 mer; 5-10-5 |
| 141 | CTCACATGGGACTCCCCAGA | Intron + Exon-1 | Full PS; 2′MOE; 20 mer |
| 142 | CTGAGAAGACAAGGAGTCCT | Exon-5 | Full PS; 2′MOE; 20 mer |
| 143 | TCTTGGTGAAGTGGATCT | Exon10_Ext | Full PS; 2′MOE; 18 mer; 4-10-4 |
| 144 | GTAGCAGCTTGTGGTTGA | Exon-4 + Intron | Full PS; 2′MOE; 18 mer |
| 145 | TGTAGTAGAATTTCTCTGTA | Exon-7 | Full PS; 2′MOE; 20 mer |
| 146 | GGTGAAGTGGATCTGGTA | | Full PS; 2′MOE; 18 mer; 4-10-4, PO after 2nd |
| | | | from 5′ end, PO after 3rd position from 3′ end |
| 147 | CTTTTCTGACTTGAACTCCC | Exon-3 | Full PS; 2′MOE; 20 mer |
| 148 | GTGTAGATGGTCTTGTCTGT | Exon-3 + Intron | Full PS; 2′MOE; 20 mer; 5-10-5, PO after 2nd |
| | | | base from 5′ end, PO after 3rd base from 3′ end |
| 149 | GGGACCCACCTTGAGCTCTT | Exon20_Ext | Full PS; 2′MOE; 20 mer; 5-10-5, PO after 2nd |
| | | | base from 5′ end, PO after 3rd base from 3′ end |
| 150 | CATACTTGACGAGTTCCGGA | Intron + Exon-5 | Full PS; 2′MOE; 20 mer |
| 151 | AAGGAGTCCTGCTTGACCGG | Exon-5 | Full PS; 2′MOE; 20 mer |
| 152 | CTCGGATCTTCCACTGGCCC | Exon-6 | Full PS; 2′MOE; 20 mer |
| 153 | TTATAGATGTAGTAGAAT | Exon-7 + Intron | Full PS; 2′MOE; 18 mer |
| 154 | GCATTGTTTCTGGTTCTCTT | Exon | Full PS; 2′MOE; 20 mer; 5-10-5 |
| 155 | AGTAGAATTTCTCTGTAG | Exon-7 + Intron | Full PS; 2′MOE; 18 mer |
| 156 | GTCTGGATGAAGAGGTACCC | Exon-3 + Intron | Full PS; 2′MOE; 20 mer; 5-10-5, PO after 2nd |
| | | | base from 5′ end, PO after 3rd base from 3′ end |
| 157 | GTCTTCTCACTGGACAGCAC | Exon-2 | Full PS; 2′MOE; 20 mer |
| 158 | GCAGAAAGAAGGGCATTGG | Exon34_Ext | Full PS; 2′MOE; 20 mer; 5-10-5, PO after 2nd |
| | G | | base from 5′ end, PO after 3rd base from 3′ end |
| 159 | TCATAGTAGGCTCGGATCTT | Exon-6 | Full PS; 2′MOE; 20 mer |
| 160 | ACAGTGTAGGATCTCTGTAG | | Full PS; 2′MOE; 20 mer; 5-10-5, PO after 2nd |
| | | | base from 5′ end, PO after 3rd base from 3′ end |
| 161 | CTGGATGAAGAGGTACCC | | Full PS; 2′MOE; 18 mer; 4-10-4, PO after 2nd |
| | | | from 5′ end, PO after 3rd position from 3′ end |
| 162 | TAGATGGTCTTGTCTGTCTG | Exon | Full PS; 2′MOE; 20 mer; 5-10-5 |
| 163 | CTTGGTGAAGTGGATCTG | Exon10_Ext | Full PS; 2′MOE; 18 mer |
| 164 | ACCATGAGGTCAAAGGGCAT | Exon10_Ext | Full PS; 2′MOE; 20 mer; 5-10-5, PO after 2nd |
| | | | base from 5′ end, PO after 3rd base from 3′ end |
| 165 | ATGCCTTCCGGGTTCTGTGG | Exon-5 + Intron | Full PS; 2′MOE; 20 mer |
| 166 | TCTTGGTGAAGTGGATCTGG | | Full PS; 2′MOE; 20 mer; 5-10-5; PO after 2nd |
| | | | from 5′ end, PO after 3rd position from 3′ end |
| 167 | TGGTGACCTCCAGGCCCTTC | Exon-7 | Full PS; 2′MOE; 20 mer |
| 168 | GCTCAAGGGCTTCTGGCTGG | Exon11_Ext | Full PS; 2′MOE; 20 mer; 5-10-5, PO after 2nd |
| | | | base from 5′ end, PO after 3rd base from 3′ end |
| 169 | CTGGCGGTGATGGTGACCTC | Exon-7 | Full PS; 2′MOE; 20 mer |
| 170 | TCTGTCTGGATGAAGAGGTA | | Full PS; 2′MOE; 20 mer; 5-10-5; PO after 2nd |
| | | | from 5′ end, PO after 3rd position from 3′ end |
| 171 | GAAGATGACAAAGGCAGTTC | | Full PS; 2′MOE; 20 mer; 5-10-5, PO after 2nd |
| | | | base from 5′ end, PO after 3rd base from 3′ end |
| 172 | GAGGTACCCGCTCTGCAGGC | Exon-3 | Full PS; 2′MOE; 20 mer |
| 173 | TCTGGATGAAGAGGTACCCG | Exon-3 | Full PS; 2′MOE; 20 mer |
| 174 | CTCACCGTGAAGGTGACGTT | Intron + Exon-2 | Full PS; 2′MOE; 20 mer |
| 175 | ATGAAGGTGGCCTAGAACCC | Exon30_Ext | Full PS; 2′MOE; 20 mer; 5-10-5, PO after 2nd |
| | | | base from 5′ end, PO after 3rd base from 3′ end |
| 176 | CCAGCCTCACCTGTGGAGCC | Intron + Exon-3 | Full PS; 2′MOE; 20 mer |
| 177 | TGGTGAAGTGGATCTGGT | | Full PS; 2′MOE; 18 mer; 4-10-4, PO after 2nd |
| | | | from 5′ end, PO after 3rd position from 3′ end |
| 178 | TTGGTGAAGTGGATCTGG | | Full PS; 2′MOE; 18 mer; 4-10-4, PO after 2nd |
| | | | from 5′ end, PO after 3rd position from 3′ end |
| 179 | AACCACTCACATGGGACTCC | Intron + Exon-1 | Full PS; 2′MOE; 20 mer |
| 180 | TGGTGAAGTGGATCTGGT | Exon10_Ext | Full PS; 2′MOE; 18 mer |
| 181 | GATTCAGGCAGGGAAATCCT | Exon | Full PS; 2′MOE; 20 mer; 5-10-5 |
| 182 | TAGGTTCATGTAGTTGGC | Exon28_Ext | Full PS; 2′MOE; 18 mer |
| 183 | TGGATGAAGAGGTACCCG | | Full PS; 2′MOE; 18 mer; 4-10-4, PO after 2nd |
| | | | from 5′ end, PO after 3rd position from 3′ end |
| 184 | GTCCCGAAGGTGGCCTGCAC | Exon-3 | Full PS; 2′MOE; 20 mer |
| 185 | TCTCCTCGCTCTCCAGCCGC | Exon-2 | Full PS; 2′MOE; 20 mer |
| 186 | GGAACTCACCCTCAGCACAG | Exon37_Ext | Full PS; 2′MOE; 20 mer; 5-10-5, PO after 2nd |
| | | | base from 5′ end, PO after 3rd base from 3′ end |
| 187 | TTCCCGATGATGTAGCTGAG | Exon | Full PS; 2′MOE; 20 mer; 5-10-5 |
| 188 | TCGAAACTGGGCAGCACTGG | Exon-7 + Intron | Full PS; 2′MOE; 20 mer |
| 189 | TGGTCTTGTCTGTCTGGATG | Exon-3 + Intron | Full PS; 2′MOE; 20 mer; 5-10-5, PO after 2nd |
| | | | base from 5′ end, PO after 3rd base from 3′ end |
| 190 | GTTCTGAGAAGACAAGGAGT | | Full PS; 2′MOE; 20 mer; 5-10-5, PO after 2nd |
| | | | base from 5′ end, PO after 3rd base from 3′ end |
| 191 | CTTGGTGAAGTGGATCTG | | Full PS; 2′MOE; 18 mer; 4-10-4, PO after 2nd |
| | | | from 5′ end, PO after 3rd position from 3′ end |
| 192 | GTGGGAACAGGTGAGGTTTC | ENST00000245 | 20 mer; Gapmer; 5-10-5, PO after 2nd base |
| | | 907.11_utr3_40 | from 5′ end, PO after 3rd base from 3′ end; Full |
| | | 0_chr19_6677 | PS; 2′MOE |
| | | 704_r | |
| 193 | CCCACATGAGGTAGTGTTTC | ENST00000245 | 20 mer; Gapmer; 5-10-5, PO after 2nd base |
| | | 907.11_exon_3 | from 5′ end, PO after 3rd base from 3′ end: Full |
| | | 9_0_chr19_667 | PS; 2′MOE |
| | | 8152_r | |
| 194 | TTGTATGAAGCAATTCTCTG | ENST00000245 | 20 mer; Gapmer; 5-10-5, PO after 2nd base |
| | | 907.11_exon_3 | from 5′ end, PO after 3rd base from 3′ end: Full |
| | | | PS; 2′MOE |
| | | 7_0_chr19_667 | |
| | | 9125_r | |
| 195 | GCTGGATAAGCTCTACATTA | ENST00000245 | 20 mer; Gapmer; 5-10-5, PO after 2nd base |
| | | 907.11_exon_3 | from 5′ end, PO after 3rd base from 3′ end: Full |
| | | 5_0_chr19_668 | PS; 2′MOE |
| | | 0158_r | |
| 196 | GCCAGTCATCATGGATATGT | ENST00000245 | 20 mer; Gapmer; 5-10-5, PO after 2nd base |
| | | 907.11_exon_3 | from 5′ end, PO after 3rd base from 3′ end: Full |
| | | 3_0_chr19_668 | PS; 2′MOE |
| | | 2142_r | |
| 197 | GATCATAGTGTTCTTGGCAT | ENST00000245 | 20 mer; Gapmer; 5-10-5, PO after 2nd base |
| | | 907.11_exon_3 | from 5′ end, PO after 3rd base from 3′ end: Full |
| | | 2_0_chr19_668 | PS; 2′MOE |
| | | 4388_r | |
| 198 | GCCTTAGCATGGTACATTGT | ENST00000245 | 20 mer; Gapmer; 5-10-5, PO after 2nd base |
| | | 907.11_exon_3 | from 5′ end, PO after 3rd base from 3′ end: Full |
| | | 1_0_chr19_668 | PS; 2′MOE |
| | | 4560_r | |
| 199 | GTGAGGGCCATGTCTTTCTC | ENST00000245 | 20 mer; Gapmer; 5-10-5, PO after 2nd base |
| | | 907.11_exon_2 | from 5′ end, PO after 3rd base from 3′ end: Full |
| | | 6_0_chr19_669 | PS; 2′MOE |
| | | 0629_r | |
| 200 | CCACCCTTCTTGATGAGCTC | ENST00000245 | 20 mer; Gapmer; 5-10-5, PO after 2nd base |
| | | 907.11_exon_2 | from 5′ end, PO after 3rd base from 3′ end: Full |
| | | 3_0_chr19_669 | PS; 2′MOE |
| | | 4431_r | |
| 201 | GAGAATTCTGGTCTCAGACT | ENST00000245 | 20 mer; Gapmer; 5-10-5, PO after 2nd base |
| | | 907.11_exon_2 | from 5′ end, PO after 3rd base from 3′ end: Full |
| | | 2_0_chr19_669 | PS; 2′MOE |
| | | 6379_r | |
| 202 | GCTGGATTGTGGAGTAGTTC | ENST00000245 | 20 mer; Gapmer; 5-10-5, PO after 2nd base |
| | | 907.11_exon_2 | from 5′ end, PO after 3rd base from 3′ end: Full |
| | | 0_0_chr19_669 | PS; 2′MOE |
| | | 7344_r | |
| 203 | GTGGTGATGGAGTCTTTCAA | ENST00000245 | 20 mer; Gapmer; 5-10-5, PO after 2nd base |
| | | 907.11_exon_1 | from 5′ end, PO after 3rd base from 3′ end: Full |
| | | 8_0_chr19_670 | PS; 2′MOE |
| | | 2127_r | |
| 204 | GTGGTCACCCTCTATCTTCA | ENST00000245 | 20 mer; Gapmer; 5-10-5, PO after 2nd base |
| | | 907.11_exon_1 | from 5′ end, PO after 3rd base from 3′ end: Full |
| | | 3_0_chr19_670 | PS; 2′MOE |
| | | 9684_r | |
| 205 | GACCAGGTAGGTGTAGTAGC | ENST00000245 | 20 mer; Gapmer; 5-10-5, PO after 2nd base |
| | | 907.11_exon_1 | from 5′ end, PO after 3rd base from 3′ end: Full |
| | | 1_0_chr19_671 | PS; 2′MOE |
| | | 0987_r | |
| 206 | CACCTCAAACTCAGTGGAGA | ENST00000245 | 20 mer; Gapmer; 5-10-5, PO after 2nd base |
| | | 907.11_exon_5 | from 5′ end, PO after 3rd base from 3′ end: Full |
| | | _0_chr19_6714 | PS; 2′MOE |
| | | 166 r | |
| 207 | GGTAGCAGCTTGTGGTTGAC | ENST00000245 | 20 mer; Gapmer; 5-10-5, PO after 2nd base |
| | | 907.11_exon_3 | from 5′ end, PO after 3rd base from 3′ end: Full |
| | | _0_chr19_6718 | PS; 2′MOE |
| | | 094_r | |
| 208 | GTCTTCTCACTGGACAGCAC | ENST00000245 | 20 mer; Gapmer; 5-10-5, PO after 2nd base |
| | | 907.11_exon_1 | from 5′ end, PO after 3rd base from 3′ end: Full |
| | | _0_chr19_6719 | PS; 2′MOE |
| | | 211_r | |
| 209 | CGGACCAGGTAGGTGTAGTA | ENST00000245 | 20 mer; Gapmer; 5-10-5, PO after 2nd base |
| | | 907.11_exon_1 | from 5′ end, PO after 3rd base from 3′ end: Full |
| | | 1_0_chr19_671 | PS; 2′MOE |
| | | 0987_r | |
| 210 | ATATAACTGAAGCTTTAT | ENST00000245 | 18 mer; Gapmer; 4-10-4, PO after 2nd from 5′ |
| | | 907.11_utr3_40 | end, PO after 3rd position from 3′ end: Full PS; |
| | | _0_chr19_6677 | 2′MOE |
| | | 704_r | |

*Gapmers are indicated by structures denoted (X)-(Y)-(X), wherein “(X)” refers to regions comprising an ‘X’ number of nucleotide positions having ribose and which flank a region referred to as “(Y)” comprising a ‘Y’ number of nucleotide positions having deoxyribose
** Skipmers (alternatively referred to herein as skippers) are indicated by the lack of notation reciting “(X)-(Y)-(X)” and each nucleotide position comprises ribose
Abbreviations:
“Full PS” refers to each nucleotide position linked by an internucleotide linkage comprising a phosphorothioate modification unless otherwise indicated by nucleotide positions comprising a phosphodiester group (PO) at the 3′ carbon of ribose
“2′MOE” refers to the 2′ carbon of ribose bonded to an oxygen atom which is bonded to a methoxyethyl group
“#mer” refers to the number of nucleotide positions
“PO” refers to a nucleotide position, wherein the 3′ carbon of ribose is linked through a phosphodiester bond which links the nucleotide position at its 5′ carbon to an immediately adjacent nucleotide position in the 3′ direction

In some embodiments, an RNA processing modulator (e.g., an antisense oligonucleotide) comprises at least 18 continuous nucleotides (e.g., comprising or consisting of 18 nucleotides, 19 nucleotides, or 20 nucleotides) of any one of the nucleic acid sequences set forth in SEQ ID NOs: 1-210 (see Column A of Table 1). In some embodiments, an RNA processing modulator consists of 18 continuous nucleotides of any one of the nucleic acid sequences set forth in Column A of Table 1 which are labeled “18mers” in Column B of the same row in Table 1. In some embodiments, an RNA processing modulator comprises 18 continuous nucleotides of any one of the nucleic acid sequences set forth in Column A of Table 1 which are labeled “18mers” in Column B of the same row in Table 1 and comprises one additional nucleotide (either at the 5′ end or 3′ end) or two additional nucleotides (either both at the 5′ end, both at the 3′ end, or one at the 5′ end and the other at the 3′ end) which are complementary to a target sequence in a C3 mRNA that hybridizes to the 18 continuous nucleotides of the nucleic acid sequence selected from Column A of Table 1. In some embodiments, an RNA processing modulator consists of 19 continuous nucleotides of any one of the nucleic acid sequences set forth in Column A of Table 1 which are labeled “20mers” in Column B of the same row in Table 1 and comprises one additional nucleotide either at the 5′ end or 3′ end which are complementary to a target sequence in a C3 mRNA that hybridizes to the 20 continuous nucleotides of the nucleic acid sequence selected from Column A of Table 1. In some embodiments, an RNA processing modulator comprises or consists of 20 continuous nucleotides of any one of the nucleic acid sequences set forth in Column A of Table 1 which are labeled “20mers” in Column B of the same row in Table 1. In some embodiments, an RNA processing modulator comprises 20 continuous nucleotides of any one of the nucleic acid sequences set forth in Column A of Table 1 which are labeled “20mers” in Column B of the same row in Table 1 and comprises one or more additional nucleotides either at the 5′ end, at the 3′ end, or both the 5′ end and the 3′ end which are complementary to a target sequence in a C3 mRNA that hybridizes to the 20 continuous nucleotides of the nucleic acid sequence selected from Column A of Table 1. In some embodiments, an RNA processing modulator comprising at least 18 continuous nucleotides of any one of the nucleic acid sequences set forth in SEQ ID NOs: 1-210 (e.g., an ASO comprising or consisting of 18 nucleotides, 19 nucleotides, or 20 continuous nucleotides of any one of the nucleic acid sequences shown in Column A of Table 1) comprises one or more chemical modifications as set forth in any one of the rows in Column B of Table 1. In some embodiments, an RNA processing modulator comprising at least 18 continuous nucleotides of any one of the nucleic acid sequences set forth in SEQ ID NOs: 1-210 (e.g., an ASO comprising or consisting of 18 nucleotides, 19 nucleotides, or 20 continuous nucleotides of any one of the nucleic acid sequences shown in Column A of Table 1) comprises a pattern of chemical modifications as set forth in any one of the rows in Column B of Table 1. In some embodiments, the at least 18 continuous nucleotides comprised in an RNA processing modulator are set forth in the nucleic acid sequence of SEQ ID NO: 16. In some embodiments, the at least 18 continuous nucleotides comprised in an RNA processing modulator are set forth in the nucleic acid sequence of SEQ ID NO: 71. In some embodiments, the at least 18 continuous nucleotides comprised in an RNA processing modulator are set forth in the nucleic acid sequence of SEQ ID NO: 156. In some embodiments, an RNA processing modulator comprising the at least 18 continuous nucleotides of any one of the nucleic acid sequences set forth in SEQ ID NOs: 1-210 reduces the levels of a C3 mRNA (e.g., a mature mRNA or a pre-mRNA) and/or a C3 protein by 50% or more (e.g., 50-60%, 60-70%, 70-80%, 80-90%, 90-95%, or 95-100%) in a cell or one or more tissues, such as a cell or one or more tissues (e.g., cerebrospinal fluid, plasma, and/or a brain tissue) in a subject when the RNA processing modulator or a composition thereof is administered to the subject in an effective amount. In some embodiments, an RNA processing modulator comprising the at least 18 continuous nucleotides of any one of the nucleic acid sequences set forth in SEQ ID NOs: 16, 71, and 156 reduces the levels of a C3 mRNA (e.g., a mature mRNA or a pre-mRNA) and/or a C3 protein by 50% or more (e.g., 50-60%, 60-70%, 70-80%, 80-90%, 90-95%, or 95-100%) in a cell or one or more tissues, such as a cell or one or more tissues (e.g., cerebrospinal fluid, plasma, and/or a brain tissue) in a subject when the RNA processing modulator or a composition thereof is administered to the subject in an effective amount.

In some embodiments, an RNA processing modulator comprises or consists of 18 continuous nucleotides, comprises or consists of 19 continuous nucleotides, or comprises or consists of 20 continuous nucleotides of any one of the nucleic acid sequences set forth in SEQ ID NOs: 1-210 (see Column A of Table 1), wherein one or more of positions comprising a “T” residue is substituted for a “U” residue. In some embodiments, an RNA processing modulator comprises or consists of 18 continuous nucleotides, comprises or consists of 19 continuous nucleotides, or comprises or consists of 20 continuous nucleotides of any one of the nucleic acid sequences set forth in SEQ ID NOs: 1-210 (see Column A of Table 1), wherein each position comprising a “T” residue is substituted for a “U” residue. In some embodiments, an RNA processing modulator comprising at least 18 continuous nucleotides of any one of the nucleic acid sequences set forth in SEQ ID NOs: 1-210 (e.g., an ASO comprising or consisting of 18 nucleotides, 19 nucleotides, or 20 continuous nucleotides of any one of the nucleic acid sequences shown in Column A of Table 1), wherein one or more of positions comprising a “T” residue is substituted for a “U” residue and wherein the RNA processing modulator comprises one or more chemical modifications as set forth in any one of the rows in Column B of Table 1. In some embodiments, an RNA processing modulator comprising at least 18 continuous nucleotides of any one of the nucleic acid sequences set forth in SEQ ID NOs: 1-210 (e.g., an ASO comprising or consisting of 18 nucleotides, 19 nucleotides, or 20 continuous nucleotides of any one of the nucleic acid sequences shown in Column A of Table 1), wherein one or more of positions comprising a “T” residue is substituted for a “U” residue and wherein the RNA processing modulator comprises a pattern of chemical modifications as set forth in any one of the rows in Column B of Table 1. In some embodiments, one or more positions in an RNA processing modulator comprising “U” residues comprises an uracil nitrogenous base or a chemically modified uracil nitrogenous base described herein and a deoxyribose sugar or a chemically modified deoxyribose sugar described herein. In some embodiments, the at least 18 continuous nucleotides comprised in an RNA processing modulator are set forth in the nucleic acid sequence of SEQ ID NO: 16, wherein one or more of positions in SEQ ID NO: 16 comprising a “T” residue (e.g., each position in SEQ ID NO: 16 comprising a “T” residue) is substituted for a “U” residue. In some embodiments, the at least 18 continuous nucleotides comprised in an RNA processing modulator are set forth in the nucleic acid sequence of SEQ ID NO: 71, wherein one or more of positions in SEQ ID NO: 71 comprising a “T” residue (e.g., each position in SEQ ID NO: 71 comprising a “T” residue) is substituted for a “U” residue. In some embodiments, the at least 18 continuous nucleotides comprised in an RNA processing modulator are set forth in the nucleic acid sequence of SEQ ID NO: 156, wherein one or more of positions in SEQ ID NO: 156 comprising a “T” residue (e.g., each position in SEQ ID NO: 156 comprising a “T” residue) is substituted for a “U” residue.

In some embodiments, an RNA processing modulator (e.g., an antisense oligonucleotide) comprises a gapmer structure, wherein a region of 10 deoxyribonucleotides is flanked by regions each comprising 4 ribonucleotides (thereby totaling 8 ribonucleotides and 10 deoxyribonucleotides). In some embodiments, 1, 2, 3, or 4 ribonucleotides in each of the regions flanking the region of 10 deoxyribonucleotides comprise a 2′-O-methoxyethyl (—OCH2CH2OCH3 (2′ MOE)) modification. In some embodiments, 1, 2, 3, or 4 ribose sugars comprised in each region flanking the region of 10 deoxyribonucleotides is linked by a phosphorothioate linkage or a phosphodiester linkage. In some embodiments, 1-10 deoxyribose sugars (e.g., 1, 2, 3, 4, 5, 6, 7, 8, 9, or 10 deoxyribose sugars) comprised in the region of 10 deoxyribonucleotides is linked by a phosphorothioate linkage. In some embodiments, one or more ribose sugars (e.g., 1, 2, 3, or 4 ribose sugars) comprised in each of the regions flanking the region of 10 deoxyribonucleotides is linked by a phosphorothioate linkage. In some embodiments, 16 out of the 18 positions are linked by phosphorothioate linkages. In some embodiments, 16 out of the 18 positions are linked by phosphorothioate linkages, wherein the second position is linked to the third position (relative to the 5′ terminal end) by a phosphodiester linkage and the sixteenth position is linked to the seventeenth position (relative to the 5′ terminal end) by a phosphodiester linkage. In some embodiments, an RNA processing modulator (e.g., an antisense oligonucleotide) comprising the gapmer structure comprises or consists of 18 continuous nucleotides of any one of the nucleic acid sequences set forth in SEQ ID NOs: 1-210 (see Column A of Table 1). In some embodiments, an RNA processing modulator (e.g., an antisense oligonucleotide) comprising the gapmer structure comprises or consists of 18 continuous nucleotides of the nucleic acid sequence of SEQ ID NO: 16. In some embodiments, an RNA processing modulator (e.g., an antisense oligonucleotide) comprising the gapmer structure comprises or consists of 18 continuous nucleotides of the nucleic acid sequence of SEQ ID NO: 71. In some embodiments, an RNA processing modulator (e.g., an antisense oligonucleotide) comprising the gapmer structure comprises or consists of 18 continuous nucleotides of the nucleic acid sequence of SEQ ID NO: 156. In some embodiments, an RNA processing modulator comprising the gapmer structure reduces the levels of a C3 mRNA (e.g., a mature mRNA or a pre-mRNA) and/or a C3 protein by 50% or more (e.g., 50-60%, 60-70%, 70-80%, 80-90%, 90-95%, or 95-100%) in a cell or one or more tissues, such as a cell or one or more tissues (e.g., cerebrospinal fluid, plasma, and/or a brain tissue) in a subject when the RNA processing modulator or a composition thereof is administered to the subject in an effective amount.

In some embodiments, an RNA processing modulator (e.g., an antisense oligonucleotide) comprises a gapmer structure, wherein a region of 10 deoxyribonucleotides is flanked by regions each comprising 5 ribonucleotides (thereby totaling 10 ribonucleotides and 10 deoxyribonucleotides). In some embodiments, 1, 2, 3, 4, or 5 ribonucleotides in each of the regions flanking the region of 10 deoxyribonucleotides comprise a 2′-O-methoxyethyl (—OCH2CH2OCH3 (2′ MOE)) modification. In some embodiments, 1, 2, 3, 4, or 5 ribose sugars comprised in each region flanking the region of 10 deoxyribonucleotides is linked by a phosphorothioate linkage or a phosphodiester linkage. In some embodiments, 1-10 deoxyribose sugars (e.g., 1, 2, 3, 4, 5, 6, 7, 8, 9, or 10 deoxyribose sugars) comprised in the region of 10 deoxyribonucleotides is linked by a phosphorothioate linkage. In some embodiments, one or more ribose sugars (e.g., 1, 2, 3, 4, or 5 ribose sugars) comprised in each of the regions flanking the region of 10 deoxyribonucleotides is linked by a phosphorothioate linkage. In some embodiments, 16 out of the 20 positions are linked by phosphorothioate linkages. In some embodiments, 16 out of the 20 positions are linked by phosphorothioate linkages, wherein the second position and third position (relative to the 5′ terminal end), third position and fourth position (relative to the 5′ terminal end), seventeenth position and eighteenth position (relative to the 5′ terminal end), and eighteenth position and nineteenth position (relative to the 5′ terminal end) are each linked by a phosphodiester linkage. In some embodiments, 18 out of the 20 positions are linked by phosphorothioate linkages. In some embodiments, 18 out of the 20 positions are linked by phosphorothioate linkages, wherein the second position and third position (relative to the 5′ terminal end) are linked by a phosphodiester linkage and the seventeenth position and eighteenth position (relative to the 5′ terminal end) are linked by a phosphodiester linkage. In some embodiments, an RNA processing modulator (e.g., an antisense oligonucleotide) comprising the gapmer structure comprises or consists of 20 continuous nucleotides of any one of the nucleic acid sequences set forth in SEQ ID NOs: 1-210 (see Column A of Table 1). In some embodiments, an RNA processing modulator (e.g., an antisense oligonucleotide) comprising the gapmer structure comprises or consists of 20 continuous nucleotides of the nucleic acid sequence of SEQ ID NO: 71. In some embodiments, an RNA processing modulator (e.g., an antisense oligonucleotide) comprising the gapmer structure comprises or consists of 20 continuous nucleotides of the nucleic acid sequence of SEQ ID NO: 156. In some embodiments, an RNA processing modulator comprising the gapmer structure reduces the levels of a C3 mRNA (e.g., a mature mRNA or a pre-mRNA) and/or a C3 protein by 50% or more (e.g., 50-60%, 60-70%, 70-80%, 80-90%, 90-95%, or 95-100%) in a cell or one or more tissues, such as a cell or one or more tissues (e.g., cerebrospinal fluid, plasma, and/or a brain tissue) in a subject when the RNA processing modulator or a composition thereof is administered to the subject in an effective amount.

In some embodiments, an isolated nucleic acid (e.g., an RNA processing modulator, such as an antisense oligonucleotide) comprises the nucleic acid sequence set forth in SEQ ID NO: 1 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 2 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 3 and the following modification pattern: Full PS; 2′MOE; 20mer; 5-10-5; PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 4 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 5 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 6 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 7 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 8 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 9 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 10 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 11 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 12 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 13 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 14 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 15 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 16 and the following modification pattern: Full PS; 2′MOE; 18mer; 4-10-4; PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 17 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 18 and the following modification pattern: Full PS; 2′MOE; 18mer; 4-10-4, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 19 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 20 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 21 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 22 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 23 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 24 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 25 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 26 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 27 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 28 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 29 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 30 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 31 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 32 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 33 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 34 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 35 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 36 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 37 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 38 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 39 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 40 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 41 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 42 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 43 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 44 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 45 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 46 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 47 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 48 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 49 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 50 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 51 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 52 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 53 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 54 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 55 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 56 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 57 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 58 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 59 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 60 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 61 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 62 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 63 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 64 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 65 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 66 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 67 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 68 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 69 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 70 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 71 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 72 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 73 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 74 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 75 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 76 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 77 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 78 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 79 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 80 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 81 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 82 and the following modification pattern: Full PS; 2′MOE; 20mer; 5-10-5; PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 83 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 84 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 85 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 86 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 87 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 88 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 89 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 90 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 91 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 92 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 93 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 94 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 95 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 96 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 97 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 98 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 99 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 100 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 101 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 102 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 103 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 104 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 105 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 106 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 107 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 108 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 109 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 110 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 111 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 112 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 113 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 114 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 115 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 116 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 117 and the following modification pattern: Full PS; 2′MOE; 16mer. In some embodiments, an isolated nucleic acid (e.g., an RNA processing modulator, such as an antisense oligonucleotide) comprises the nucleic acid sequence set forth in SEQ ID NO: 118 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 119 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 120 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 121 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 122 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 123 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 124 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 125 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 126 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 127 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 128 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 129 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 130 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 131 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 132 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 133 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 134 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 135 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 136 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 137 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 138 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 139 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 140 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 141 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 142 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 143 and the following modification pattern: Full PS; 2′MOE; 18mer; 4-10-4. In some embodiments, an isolated nucleic acid (e.g., an RNA processing modulator, such as an antisense oligonucleotide) comprises the nucleic acid sequence set forth in SEQ ID NO: 144 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 145 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 146 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 147 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 148 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 149 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 150 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 151 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 152 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 153 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 154 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 155 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 156 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 157 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 158 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 159 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 160 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 161 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 162 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 163 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 164 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 165 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 166 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 167 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 168 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 169 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 170 and the following modification pattern: Full PS; 2′MOE; 20mer; 5-10-5; PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 171 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 172 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 173 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 174 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 175 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 176 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 177 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 178 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 179 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 180 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 181 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 182 and the following modification pattern: Full PS; 2′MOE; 18mer. In some embodiments, an isolated nucleic acid (e.g., an RNA processing modulator, such as an antisense oligonucleotide) comprises the nucleic acid sequence set forth in SEQ ID NO: 183 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 184 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 185 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 186 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 187 and the following modification pattern: Full PS; 2′MOE; 20mer; 5-10-5. In some embodiments, an isolated nucleic acid (e.g., an RNA processing modulator, such as an antisense oligonucleotide) comprises the nucleic acid sequence set forth in SEQ ID NO: 188 and the following modification pattern: Full PS; 2′MOE; 20mer. In some embodiments, an isolated nucleic acid (e.g., an RNA processing modulator, such as an antisense oligonucleotide) comprises the nucleic acid sequence set forth in SEQ ID NO: 189 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 190 and the following modification pattern: Full PS; 2′MOE; 20mer; 5-10-5, PO after 2nd base from 5′ end, PO after 3rd base from 3′ end. In some embodiments, an isolated nucleic acid (e.g., an RNA processing modulator, such as an antisense oligonucleotide) comprises the nucleic acid sequence set forth in SEQ ID NO: 191 and the following modification pattern: Full PS; 2′MOE; 18mer; 4-10-4, PO after 2nd from 5′ end, PO after 3rd position from 3′ end. In some embodiments, an isolated nucleic acid (e.g., an RNA processing modulator, such as an antisense oligonucleotide) comprises the nucleic acid sequence set forth in SEQ ID NO: 192 and the following modification pattern: 20mer; Gapmer; 5-10-5, PO after 2nd base from 5′ end, PO after 3rd base from 3′ end; Full PS; 2′MOE. In some embodiments, an isolated nucleic acid (e.g., an RNA processing modulator, such as an antisense oligonucleotide) comprises the nucleic acid sequence set forth in SEQ ID NO: 193 and the following modification pattern: 20mer; Gapmer; 5-10-5, PO after 2nd base from 5′ end, PO after 3rd base from 3′ end: Full PS; 2′MOE. In some embodiments, an isolated nucleic acid (e.g., an RNA processing modulator, such as an antisense oligonucleotide) comprises the nucleic acid sequence set forth in SEQ ID NO: 194 and the following modification pattern: 20mer; Gapmer; 5-10-5, PO after 2nd base from 5′ end, PO after 3rd base from 3′ end: Full PS; 2′MOE. In some embodiments, an isolated nucleic acid (e.g., an RNA processing modulator, such as an antisense oligonucleotide) comprises the nucleic acid sequence set forth in SEQ ID NO: 195 and the following modification pattern: 20mer; Gapmer; 5-10-5, PO after 2nd base from 5′ end, PO after 3rd base from 3′ end: Full PS; 2′MOE. In some embodiments, an isolated nucleic acid (e.g., an RNA processing modulator, such as an antisense oligonucleotide) comprises the nucleic acid sequence set forth in SEQ ID NO: 196 and the following modification pattern: 20mer; Gapmer; 5-10-5, PO after 2nd base from 5′ end, PO after 3rd base from 3′ end: Full PS; 2′MOE. In some embodiments, an isolated nucleic acid (e.g., an RNA processing modulator, such as an antisense oligonucleotide) comprises the nucleic acid sequence set forth in SEQ ID NO: 197 and the following modification pattern: 20mer; Gapmer; 5-10-5, PO after 2nd base from 5′ end, PO after 3rd base from 3′ end: Full PS; 2′MOE. In some embodiments, an isolated nucleic acid (e.g., an RNA processing modulator, such as an antisense oligonucleotide) comprises the nucleic acid sequence set forth in SEQ ID NO: 198 and the following modification pattern: 20mer; Gapmer; 5-10-5, PO after 2nd base from 5′ end, PO after 3rd base from 3′ end: Full PS; 2′MOE. In some embodiments, an isolated nucleic acid (e.g., an RNA processing modulator, such as an antisense oligonucleotide) comprises the nucleic acid sequence set forth in SEQ ID NO: 199 and the following modification pattern: 20mer; Gapmer; 5-10-5, PO after 2nd base from 5′ end, PO after 3rd base from 3′ end: Full PS; 2′MOE. In some embodiments, an isolated nucleic acid (e.g., an RNA processing modulator, such as an antisense oligonucleotide) comprises the nucleic acid sequence set forth in SEQ ID NO: 200 and the following modification pattern: 20mer; Gapmer; 5-10-5, PO after 2nd base from 5′ end, PO after 3rd base from 3′ end: Full PS; 2′MOE. In some embodiments, an isolated nucleic acid (e.g., an RNA processing modulator, such as an antisense oligonucleotide) comprises the nucleic acid sequence set forth in SEQ ID NO: 201 and the following modification pattern: 20mer; Gapmer; 5-10-5, PO after 2nd base from 5′ end, PO after 3rd base from 3′ end: Full PS; 2′MOE. In some embodiments, an isolated nucleic acid (e.g., an RNA processing modulator, such as an antisense oligonucleotide) comprises the nucleic acid sequence set forth in SEQ ID NO: 202 and the following modification pattern: 20mer; Gapmer; 5-10-5, PO after 2nd base from 5′ end, PO after 3rd base from 3′ end: Full PS; 2′MOE. In some embodiments, an isolated nucleic acid (e.g., an RNA processing modulator, such as an antisense oligonucleotide) comprises the nucleic acid sequence set forth in SEQ ID NO: 203 and the following modification pattern: 20mer; Gapmer; 5-10-5, PO after 2nd base from 5′ end, PO after 3rd base from 3′ end: Full PS; 2′MOE. In some embodiments, an isolated nucleic acid (e.g., an RNA processing modulator, such as an antisense oligonucleotide) comprises the nucleic acid sequence set forth in SEQ ID NO: 204 and the following modification pattern: 20mer; Gapmer; 5-10-5, PO after 2nd base from 5′ end, PO after 3rd base from 3′ end: Full PS; 2′MOE. In some embodiments, an isolated nucleic acid (e.g., an RNA processing modulator, such as an antisense oligonucleotide) comprises the nucleic acid sequence set forth in SEQ ID NO: 205 and the following modification pattern: 20mer; Gapmer; 5-10-5, PO after 2nd base from 5′ end, PO after 3rd base from 3′ end: Full PS; 2′MOE. In some embodiments, an isolated nucleic acid (e.g., an RNA processing modulator, such as an antisense oligonucleotide) comprises the nucleic acid sequence set forth in SEQ ID NO: 206 and the following modification pattern: 20mer; Gapmer; 5-10-5, PO after 2nd base from 5′ end, PO after 3rd base from 3′ end: Full PS; 2′MOE. In some embodiments, an isolated nucleic acid (e.g., an RNA processing modulator, such as an antisense oligonucleotide) comprises the nucleic acid sequence set forth in SEQ ID NO: 207 and the following modification pattern: 20mer; Gapmer; 5-10-5, PO after 2nd base from 5′ end, PO after 3rd base from 3′ end: Full PS; 2′MOE. In some embodiments, an isolated nucleic acid (e.g., an RNA processing modulator, such as an antisense oligonucleotide) comprises the nucleic acid sequence set forth in SEQ ID NO: 208 and the following modification pattern: 20mer; Gapmer; 5-10-5, PO after 2nd base from 5′ end, PO after 3rd base from 3′ end: Full PS; 2′MOE. In some embodiments, an isolated nucleic acid (e.g., an RNA processing modulator, such as an antisense oligonucleotide) comprises the nucleic acid sequence set forth in SEQ ID NO: 209 and the following modification pattern: 20mer; Gapmer; 5-10-5, PO after 2nd base from 5′ end, PO after 3rd base from 3′ end: Full PS; 2′MOE. In some embodiments, an isolated nucleic acid (e.g., an RNA processing modulator, such as an antisense oligonucleotide) comprises the nucleic acid sequence set forth in SEQ ID NO: 210 and the following modification pattern: 18mer; Gapmer; 4-10-4, PO after 2nd from 5′ end, PO after 3rd position from 3′ end: Full PS; 2′MOE. In any of the foregoing embodiments: positions comprising “T” residues comprise a thymine (T) nitrogenous base bonded to the 1′ carbon of either ribose or deoxyribose; isolated nucleic acids comprising a gapmer structure are indicated by structures denoted “(X)—(Y)—(X)”, wherein “(X)” refers to regions comprising an ‘X’ number of nucleotide positions having ribose and which flank a region referred to as “(Y)” comprising a ‘Y’ number of nucleotide positions having deoxyribose; isolated nucleic acids comprising a skipmer structure (alternatively referred to herein as skippers) are indicated by the lack of notation reciting “(X)—(Y)—(X)” and each nucleotide position comprises ribose; “Full PS” refers to each nucleotide position linked by an internucleotide linkage comprising a phosphorothioate modification unless otherwise indicated by nucleotide positions comprising a phosphodiester group (PO) at the 3′ carbon of ribose; “2′MOE” refers to the 2′ carbon of ribose bonded to an oxygen atom which is bonded to a methoxyethyl group; “#mer” refers to the number of nucleotide positions; and “PO” refers to nucleotide positions, wherein the 3′ carbon of ribose comprises a phosphodiester bond which links the nucleotide position to an immediately adjacent nucleotide position in the 3′ direction, wherein nucleotide positions comprising a PO group (e.g., a position referred to as 2nd or 3rd from a 5′ or 3′ terminal end) at the 3′ carbon of ribose is indicated by the location of the PO group which is referred to either as being “at” or “after”

### Pharmaceutical Compositions

In some embodiments of the disclosure, RNA processing modulators (e.g., antisense oligonucleotides) are formulated into compositions for therapeutic purposes. In some embodiments, the compositions are designed to enhance the therapeutic effect of the RNA processing modulators, for example by increasing biocompatibility, targeting the RNA processing modulator to a site of interest in vivo, reducing clearance of an isolated nucleic acid (e.g., an antisense oligonucleotide) in vivo, increasing the stability of an isolated nucleic acid (e.g., an antisense oligonucleotide) in vivo, increasing uptake of an isolated nucleic acid (e.g., an antisense oligonucleotide) in target cells, or amplifying the intended effect of an isolated nucleic acid (e.g., an antisense oligonucleotide) in vivo.

In some embodiments, the RNA processing modulator (e.g., antisense oligonucleotide) is provided in combination with a pharmaceutically acceptable carrier. A “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

### Methods and Medical Uses

Aspects of the disclosure relate to methods of modulating transcription, translation, function, or activity of genes associated with neuroinflammation (e.g., iatrogenic neuroinflammation associated with AAV administration, ASO administration, or antibody administration (such as Amyloid-Related Imaging Abnormalities (ARIA), for example, Amyloid-Related Imaging Abnormalities edema (ARIA-E) neuroinflammation), etc.) in a subject. Also provided is an RNA processing modulator (e.g., antisense oligonucleotide) described by the disclosure for use as a medicament. The RNA processing modulators (e.g., antisense oligonucleotides) may be used in methods of modulating transcription, translation, function, or activity of genes associated with neuroinflammation (e.g., iatrogenic neuroinflammation associated with AAV administration, ASO administration, or antibody administration (such as Amyloid-Related Imaging Abnormalities (ARIA), for example, Amyloid-Related Imaging Abnormalities edema (ARIA-E) neuroinflammation), etc.), e.g. in a cell or subject. In some embodiments, the methods comprise administering a composition comprising one or more RNA processing modulators (e.g., 1, 2, 3, 4, 5, or more RNA processing modulators, for example 1, 2, 3, 4, 5, or more antisense oligonucleotides) to a cell or subject. In some embodiments, administration of the compositions (e.g., RNA processing modulators) results in alteration of C3 levels and/or neuroinflammation in the cell or subject. The cell may be in vivo, ex vivo, or in vitro.

For example, in some embodiments, administration of an RNA processing modulator (e.g., an antisense oligonucleotide) targeting C3 mRNA results in an increase in production of C3 the cell or subject. In some embodiments, administration of an RNA processing modulator (e.g., an antisense oligonucleotide) targeting C3 mRNA results in a decrease in production of C3 in the cell or subject. The disclosure is based, in part, on the recognition that contacting a cell or subject with an RNA processing modulator that decreases transcription, translation, function or activity of C3 protein results in decreased neuroinflammation in the subject.

Accordingly, in some aspects, the disclosure provides a method for decreasing neuroinflammation in a cell or subject, the method comprising administering an isolated nucleic acid as described herein to a subject in need thereof. In some embodiments, the isolated nucleic acid comprises an antisense oligonucleotide comprising the sequence set forth in any one of SEQ ID NOs: 1-210 (provided in Column A of Table 1, optionally comprising one or more modifications in Column C of Table 1, and optionally wherein the sequence in Column A and the chemistry in Column C are provided in the same row of Table 1). In some embodiments, the isolated nucleic acid (e.g., antisense oligonucleotide) is administered as a monotherapy. In some embodiments, the isolated nucleic acid (e.g., antisense oligonucleotide) is administered as a component of a combination therapy with one or more additional therapeutic agents.

Generally, it is desirable to decrease C3 levels or neuroinflammation in certain subjects (e.g., subjects having dry age-related macular degeneration (dry AMD) (e.g., dry AMD with geographic atrophy), multiple sclerosis (MS), peripheral neuropathies (e.g., Guillain Barr syndrome (GBS), Chronic inflammatory demyelinating polyneuropathy (CIDP), etc.), neuromyelitis optica (NO), myasthenia gravis (MG), Alzheimer's disease, frontotemporal dementia (FTD), iatrogenic neuroinflammation (e.g., inflammation associated with adeno-associated virus administration, ASO administration, antibody administration (such as Amyloid-Related Imaging Abnormalities (ARIA), for example, Amyloid-Related Imaging Abnormalities edema (ARIA-E) neuroinflammation), etc.), and acute neuronal injury (e.g., traumatic brain injury (TBI), spinal cord injury, and stroke), etc.). However, it should be appreciated that, in some embodiments, the disclosure provides a method for increasing C3 levels or function or activity in a cell or subject, the method comprising administering an isolated nucleic acid as described herein to a subject in need thereof. In some embodiments, the isolated nucleic acid comprises an antisense oligonucleotide comprising the sequence set forth in any one of SEQ ID NOs: 1-210 (provided in Column A of Table 1, optionally comprising one or more modifications in Column C of Table 1, and optionally wherein the sequence in Column A and the chemistry in Column C are provided in the same row of Table 1).

In some embodiments, an isolated nucleic acid (e.g., antisense oligonucleotide) binds to an mRNA expressed from a particular allele of C3 (e.g., binds to a target mRNA in an allele-specific manner).

In some aspects, RNA processing modulators (e.g., antisense oligonucleotides) described by the disclosure are useful for treating a disease or disorder associated with neuroinflammation. Thus, provided herein are RNA processing modulators (e.g., antisense oligonucleotides) described by the disclosure for use in a method of treating a disease or disorder associated with neuroinflammation. A disease or disorder associated with neuroinflammation refers to a disease or disorder in which the subject (e.g., patient) is 1) characterized as having neuroinflammation, and/or 2) has one or more mutations in one or more genes associated with neuroinflammation.

Methods of measuring levels of inflammatory markers in cells or a subject are known. For example, C3 serum levels may be measured by an immunoassay (e.g., ELISA). In some embodiments, serum C3 level of a subject is determined by measuring the concentration of C3 in a biological sample obtained from the subject, for example a blood sample, serum sample, cerebrospinal fluid (CSF) sample, etc.

In some embodiments, a gene associated with neuroinflammation is C3. In some embodiments, a subject having a disease or disorder associated with neuroinflammation comprises one or more mutations in a C3 gene. Methods of detecting mutations in a subject's genes are known in the art and include, for example DNA sequencing, RNA sequencing, microarray analysis, etc.

In some embodiments, a subject having a disease or disorder associated with neuroinflammation comprises one or more mutations in one or more other genes that are involved in neuroinflammation. Examples of other genes involved in inflammation include other complement components (e.g., C5, C7, C8, C9), progranulin (PGRN), etc.

Accordingly, in some aspects, the disclosure provides a method for treating a disease or disorder associated with neuroinflammation, the method comprising administering an isolated nucleic acid as described herein to a subject in need thereof. Also provided is an RNA processing modulator (e.g., antisense oligonucleotide) described by the disclosure for use in a method of treating a disease or disorder associated with neuroinflammation. The method may comprise administering an isolated nucleic acid as described herein to a subject in need thereof. In some embodiments, the isolated nucleic acid comprises an antisense oligonucleotide comprising the sequence set forth in any one of SEQ ID NOs: 1-210 (provided in Column A of Table 1, optionally comprising one or more modifications in Column C of Table 1, and optionally wherein the sequence in Column A and the chemistry in Column C are provided in the same row of Table 1). In some embodiments, the disease is selected from dry age-related macular degeneration (dry AMD) (e.g., dry AMD with geographic atrophy), multiple sclerosis (MS), peripheral neuropathies (e.g., Guillain Barr syndrome (GBS), Chronic inflammatory demyelinating polyneuropathy (CIDP), etc.), neuromyelitis optica (NO), myasthenia gravis (MG), Alzheimer's disease, frontotemporal dementia (FTD), iatrogenic neuroinflammation (e.g., inflammation associated with adeno-associated virus administration, ASO administration, antibody administration (such as Amyloid-Related Imaging Abnormalities (ARIA), for example, Amyloid-Related Imaging Abnormalities edema (ARIA-E) neuroinflammation), etc.), and acute neuronal injury (e.g., traumatic brain injury (TBI), spinal cord injury, stroke, etc.).

Aspects of the disclosure relate to methods for treating iatrogenic neuroinflammation in a subject. In some embodiments, the iatrogenic neuroinflammation is caused by a complement-mediated immune reaction resulting from administration of certain therapeutic agents or procedures, for example vaccines, tumor-necrosis-factor-alpha inhibitors (TNFAIs), immune-checkpoint inhibitors (ICIs), immunomodulators, certain viral vectors (e.g., AAV vectors, lentiviral vectors, etc.), or radiation therapy. In some embodiments, iatrogenic neuroinflammation in a subject results from administration of an AAV vector. In some embodiments, iatrogenic neuroinflammation in a subject results from administration of an ASO. In some embodiments, iatrogenic neuroinflammation in a subject results from radiation therapy. It has been previously observed that, in the context of pediatric radiation therapy, reduction of C3 improves not only neuroinflammation but also prevents certain cognitive deficits associated with the neuroinflammation, for example as described by Kalm et al. Oncotarget. 2016 Apr. 12; 7(15):19382-94. doi: 10.18632/oncotarget.8400; and Markarian et al. Cancer Res. 2021 Apr. 1; 81(7):1732-1744. doi: 10.1158/0008-5472.CAN-20-2565. In some embodiments, iatrogenic neuroinflammation in a subject results from administration of an antibody to the subject (e.g., as seen in Amyloid-Related Imaging Abnormalities (ARIA), for example, Amyloid-Related Imaging Abnormalities edema (ARIA-E) neuroinflammation). In some embodiments, the antibody is aducanumab.

Aspects of the disclosure relate to methods for treating neuroinflammation in a subject having a progranulin deficiency. In some embodiments, the subject has one or more mutations, insertions, or deletions in a progranulin (PGRN) gene. In some embodiments, the subject having a progranulin deficiency has frontotemporal dementia (FTD).

As used herein “treat” or “treating” refers to preventing or delaying disease onset, reducing or preventing the development of symptoms associated with a disease, reducing the severity of a disease, and/or preventing the worsening of symptoms associated with a disease. Accordingly, in some aspects, the disclosure provides a method for treating a subject having or suspected of having a disease caused by neuroinflammation. Treatment of a subject involves administration of a composition to the subject (e.g., an RNA processing modulator, such as an antisense oligonucleotide) as described herein.

As used herein, the term “treating” refers to the application or administration of a composition (e.g., an RNA processing modulator, such as an antisense oligonucleotide as described herein) to a subject who has a disease or disorder associated with neuroinflammation, with the purpose to cure, heal, alleviate, relieve, alter, remedy, ameliorate, improve, or affect the disorder, the symptom of the disease, or the predisposition toward a disease associated with neuroinflammation.

Alleviating a disease associated with neuroinflammation includes preventing or delaying the development or progression of the disease, or reducing disease severity. Alleviating the disease does not necessarily require curative results. As used therein, “delaying” the development of a disease (such as a disease associated with neuroinflammation) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease associated with neuroinflammation.

A subject may be a human, a mouse, a rat, a pig, a dog, a cat, or a non-human primate. In some embodiments, a subject has or is suspected of having a disease or disorder associated with neuroinflammation. In some embodiments, a subject having a disease or disorder associated with neuroinflammation comprises at least one C3 allele having a mutation (e.g., a loss-of-function or a mutation that causes aberrant C3 function or activity). In some embodiments, a C3 allele having a mutation comprises a frameshift mutation, a splice site mutation, a missense mutation, a truncation mutation or a nonsense mutation. A subject may have two C3 alleles having the same mutations (homozygous state) or two C3 alleles having different mutations (compound heterozygous state).

The optimal course of administration or delivery of the RNA processing modulators (e.g., antisense oligonucleotides) of the disclosure may vary depending upon the desired result and/or on the subject to be treated. As used herein “administration” refers to contacting cells with an RNA processing modulator and can be performed in vitro or in vivo. Compositions (e.g., pharmaceutical compositions) provided herein can be administered a number of routes including, but not limited to, by oral administration, intravenous administration (e.g., systemic intravenous injection/administration), administration to the brain and/or spinal cord, intracerebral injection, intraventricular injection, intracerebroventricular (ICV) injection, intracisternal injection, intraparenchymal injection, intrathecal injection, and any combination of the foregoing. In some embodiments, administration comprises administration to cerebral spinal fluid, and/or direct administration to an affected site (e.g., a target tissue, for example eye tissue, central nervous system (CNS) tissue, or peripheral nervous system (PNS) tissue, or liver tissue).

In general, the most appropriate route of administration will depend upon a variety of factors including the nature of the agent (e.g., its stability in the environment of the gastrointestinal tract), and/or the condition of the subject (e.g., whether the subject is able to tolerate oral administration, injection, etc.). In certain embodiments, a compound or pharmaceutical composition described herein is suitable for topical administration to the eye of a subject. In some embodiments, administration (e.g., injection) of a compound or pharmaceutical composition is performed on a patient in a Trendelenburg position. In some embodiments, compositions are administered to a subject through only one administration route. In some embodiments, multiple administration routes may be exploited (e.g., serially, or simultaneously) for administration of the composition to a subject.

In some embodiments, it may be desirable to deliver the RNA processing modulators (e.g., antisense oligonucleotides) of the disclosure to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. RNA processing modulators (e.g., antisense oligonucleotides) of the disclosure may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000). In some embodiments, RNA processing modulators (e.g., antisense oligonucleotides) of the disclosure are administered by intravenous injection. In some embodiments, the RNA processing modulators (e.g., antisense oligonucleotides) of the disclosure are administered by intracerebral injection. In some embodiments, the RNA processing modulators (e.g., antisense oligonucleotides) of the disclosure are administered by intracerebroventricular (ICV) injection. In some embodiments, the RNA processing modulators (e.g., antisense oligonucleotides) of the disclosure are administered by intrathecal injection. In some embodiments, the RNA processing modulators (e.g., antisense oligonucleotides) of the disclosure are administered by intrastriatal injection. In some embodiments, the RNA processing modulators (e.g., antisense oligonucleotides) of the disclosure are delivered by intracranial injection. In some embodiments, the RNA processing modulators (e.g., antisense oligonucleotides) of the disclosure are delivered by cisterna magna injection. In some embodiments, the RNA processing modulators (e.g., antisense oligonucleotides) of the disclosure are delivered by cerebral lateral ventricle injection. The skilled artisan will also recognize that the foregoing administration routes may be combined in a single subject (e.g., a subject may be administered RNA processing modulators (e.g., antisense oligonucleotides) of the disclosure using a combination of two or more of the foregoing techniques).

In some embodiments, administration of RNA processing modulators (e.g., antisense oligonucleotides) of the disclosure results in delivery of RNA processing modulators (e.g., antisense oligonucleotides) to ocular tissue. Delivery of the RNA processing modulators (e.g., antisense oligonucleotides) to a mammalian subject may be by, for example, intraocular injection, subretinal injection, topical administration (e.g., an eye drop), or by injection into the eye of the mammalian subject to ocular tissues (e.g., intravitreal injection). As used herein, “ocular tissues” refers to any tissue derived from or contained in the eye. Non-limiting examples of ocular tissues include neurons, retina (e.g., photoreceptor cells), sclera, choroid, retina, vitreous body, macula, fovea, optic disc, lens, pupil, iris, aqueous fluid, cornea (e.g., keratocytes, corneal endothelial cells, corneal basal cells, corneal wing cells, and corneal squamous cells), conjunctiva ciliary body, and optic nerve. The retina is located in the posterior of the eye and comprises photoreceptor cells. These photoreceptor cells (e.g., rods, cones) confer visual acuity by discerning color, as well as contrast in the visual field.

In some embodiments, an effective amount (e.g., an amount sufficient to increase transcription, translation, function, or activity of a target mRNA) is administered to a subject. In some embodiments, an effective amount of an RNA processing modulator (e.g., antisense oligonucleotide) is an amount sufficient to increase transcription, translation, function, or activity of a target mRNA (e.g., of a desired mutant, variant, and/or allele). In some embodiments, an effective amount of an RNA processing modulator (e.g., antisense oligonucleotide) is an amount sufficient to decrease transcription, translation, function, or activity of a target mRNA (e.g., of an undesired mutant, variant, and/or allele). The effective amount will depend primarily on factors such as the species, age, weight, health of the subject, and the tissue to be targeted, and may thus vary among animal and tissue. In some embodiments, an effective amount can be a combination of an effective dosage, frequency, and duration for administration.

In some embodiments, an effective amount (e.g., an amount sufficient to increase transcription, translation, function, or activity of a target mRNA or an amount sufficient to decrease transcription, translation, function, or activity of a target mRNA) is 1 ng-200 μg. In some embodiments, an effective amount (e.g., an amount sufficient to increase transcription, translation, function, or activity of a target mRNA or an amount sufficient to decrease transcription, translation, function, or activity of a target mRNA) is 1-1000 ng. In some embodiments, an effective amount of (e.g., an amount sufficient to increase transcription, translation, function, or activity of a target mRNA or an amount sufficient to decrease transcription, translation, function, or activity of a target mRNA) is 1-10, 10-50, 50-100, 100-200, 200-300, 300-500, 500-750, or 750-1000 ng. In some embodiments, an effective amount (e.g., an amount sufficient to increase transcription, translation, function, or activity of a target mRNA or an amount sufficient to decrease transcription, translation, function, or activity of a target mRNA) is 0.1 μg-100.0 μg. In some embodiments, an effective amount (e.g., an amount sufficient to increase transcription, translation, function, or activity of a target mRNA or an amount sufficient to decrease transcription, translation, function, or activity of a target mRNA) is 0.1-1.0, 1.0-5.0, 5.0-20.0, 20.0-50.0, or 50.0-100.0 μg. In some embodiments, an effective amount (e.g., an amount sufficient to increase transcription, translation, function, or activity of a target mRNA or an amount sufficient to decrease transcription, translation, function, or activity of a target mRNA) is 1 μg-200 μg. In some embodiments, an effective amount (e.g., an amount sufficient to increase transcription, translation, function, or activity of a target mRNA or an amount sufficient to decrease transcription, translation, function, or activity of a target mRNA) is 10-25, 25-50, 50-75, or 75-100 μg. In some embodiments, an effective amount (e.g., an amount sufficient to increase transcription, translation, function, or activity of a target mRNA or an amount sufficient to decrease transcription, translation, function, or activity of a target mRNA) is 0.1-1.0, 1.0-5.0, 5.0-20.0, 20.0-50.0, or 50.0-100.0 μg.

In some embodiments, an effective amount (e.g., an amount sufficient to increase transcription, translation, function, or activity of a target mRNA or an amount sufficient to decrease transcription, translation, function, or activity of a target mRNA) is 0.1-1.0, 1.0-20.0, 20.0-50.0, 50.0-200.0, or 200.0-500.0 mg.

During the course of treatment, administration of the composition may be altered or adjusted accordingly. For example, expression of the protein encoded by the nucleic acid targeted by the isolated nucleic acid of the pharmaceutical composition may be monitored to inform methods of use of the composition. Expression information may be obtained, for example, through measuring changes in the levels of the protein or RNA products of the target nucleic acid. Alternatively, sequencing analyses of the target nucleic acid may be employed to determine if expression changes include alterations in the structure or sequence of the protein or RNA product of the target nucleic acid sequence.

The amount of the composition will vary depending on a number of factors such as, but not limited to, clinical features (e.g., disease severity, rate of disease progression, physical characteristics, etc.) of a subject and the mode of administration. Accordingly, the composition may, in certain instances, be administered once or more than one to a single subject. In certain instances, the composition may be administered to the same subject through different modes or routes at different times during the treatment process.

### EXAMPLES

### Example 1: RNA Processing Modulators (RPMs)

This example describes the use of RNA Processing Modulators (RPMs) for modulating translation of one or more mRNA transcripts in a cell or subject. RPMs function by binding to a target-specific mRNA sequence and altering (e.g., upregulating or down-regulating) translation of protein of the mRNA sequence.

In some embodiments, an RPM is an antisense oligonucleotide (ASO). Antisense oligonucleotides (ASOs) typically range from about 10 to 30 nucleotides in length, and may comprise a non-natural sugar-phosphate backbone (e.g., phosphorodiamidate morpholino backbone, phosphorothioate backbone, etc.) and/or one or more modified sugar moieties (e.g., 2′-O-methoxyethyl ribose (2′-O-MOE) modifications, etc.).

In some embodiments, an RPM (e.g., an ASO) targets a structural element of an mRNA transcript, for example an untranslated region (UTR) to modulate the expression of the target (e.g., the target gene encoding the mRNA transcript) by increasing or decreasing transcription and/or translation of the protein encoded by the mRNA transcript (alternatively referred to as modulating expression in the up or the down direction). In another example, an RPM (e.g., an ASO) may target a regulatory region (and thus interfere with protein binding, such as ribosomal protein binding) of a UTR region to modulate the expression of the target in the up or the down direction. Alternatively, an RPM (e.g., an ASO) may target a splice site (e.g., a splice acceptor site or a splice donor site or one or more nucleotide positions thereof in a UTR region) to modulate the expression of the target in the up or the down direction (and thus generating novel protein variants). Additional examples of structural elements that can be targeted by RPMs (e.g., ASOs) include, but are not limited to, intronic regulatory sites, exonic regulatory sites, exon-intron boundaries, antisense binding sites of a target mRNA transcript, long-non-coding RNA (LncRNA) binding sites of a target gene, and a retained exon of a canonical mRNA.

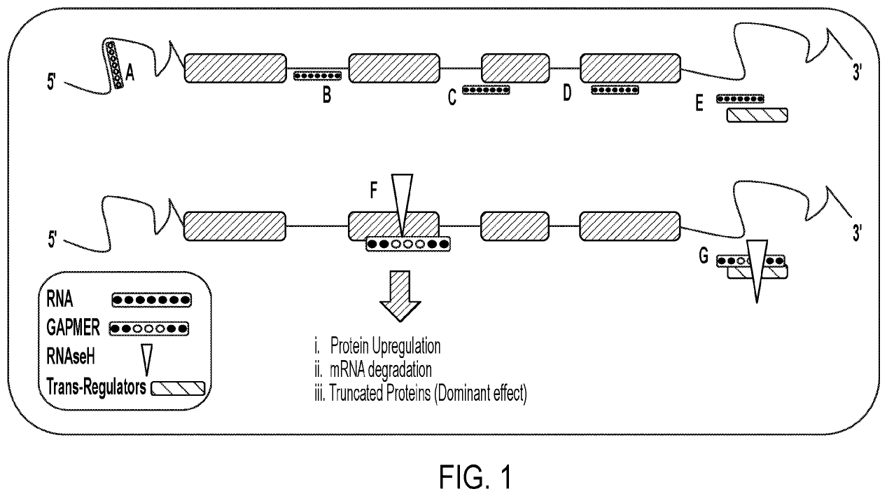

Non-limiting examples of ASOs targeting various structural elements of an mRNA are show in FIG. 1. Composition “A” represents an ASO that binds to the 5′ untranslated region (5′ UTR) of an RNA. Composition “B” represents an ASO that binds to an intron of an RNA. Composition “C” represents an ASO that binds to a splice boundary (e.g., a splice junction) between an exon and intron of an RNA. Composition “D” represents an ASO that binds to an exon (e.g., protein coding region) of an RNA. Composition “E” represents a combination of an ASO binding to a 3′ UTR of an RNA, alone or with a trans-regulator. Composition “F” represents a “gapmer” ASO that binds to an exon (e.g., a protein coding region) of an RNA and mediates RNaseH decay. Composition “G” represents a “gapmer” ASO that binds to a 3′ UTR of an RNA, alone or with a trans-regulator, and mediates RNaseH decay. In some embodiments, ASOs binding to an RNA result in translation of a truncated protein that has a dominant negative effect on the wild-type, full-length protein.

### Example 2: Neuroinflammation

This example describes diseases and disorders that area associated with neuroinflammation, and the role of Complement component C3 in such diseases and disorders.

Neuroinflammation generally refers to an innate immune system-driven inflammatory response that is centralized in the tissues of the central nervous system (CNS), for example brain and spinal cord tissue. However neuroinflammation may also encompass inflammation affecting other tissues, for example peripheral nervous system tissue (PNS) and certain cells of the eye (e.g., inflammation centralized in nervous system tissue innervating the eye). The effects of neuroinflammation are typically mediated by the production of cytokines, chemokines, reactive oxygen species, and secondary messengers from nervous system immune cells, for example glial cells (e.g., microglia) and astrocytes, and peripherally-derived immune cells. The pathophysiological effects of neuroinflammation may vary, which are dependent on the duration and severity of the inflammatory response, can lead to recruitment of immune cells, edema, tissue damage and potentially cell death.

Several diseases and disorders are associated with neuroinflammation, including neurodegenerative disease, eye disease, and certain peripheral neuropathies. For example, chronic neuroinflammation in the brain has been associated with certain neurodegenerative diseases, such as Alzheimer's disease and multiple sclerosis (MS). An injury to the head of a subject may cause a traumatic brain injury (TBI), in which an acute neuroinflammatory response may cause cell death, DNA fragmentation, and may lead to compromise of the blood-brain barrier. In another example, neuroinflammation in the optic nerve of a subject may cause cell death and lead to progressive degenerative diseases, for example age-related macular degeneration (particularly “wet” AMD), or neuromyelitis optica. Neuroinflammation in peripheral nerve tissue may lead to chronic inflammatory demyelinating polyneuropathy (CIDP).

Neuroinflammation can have many causes. For example, neuroinflammation may be caused by an injury, peripheral immune response, certain infections (e.g., infections caused by pathogens that enter the CNS or PNS), ageing, and neurodegenerative disease. Underlying each of these causes, however, is the chronic activation of a subject's innate immune system.

One key component of the innate immune system is the complement system, which is also known as complement cascade. The complement system is a part of the innate immune system that enhances the ability of antibodies and phagocytic cells to clear microbes and damaged cells from an organism, promote inflammation, and attack the pathogen's cell membrane. The complement system is activated by one of three pathways: classical, lectin, or alternative pathway. However, all of the activation pathways share a common terminal pathway that culminates in formation of the cytolytic membrane attack complex (MAC). A key regulator of the MAC complex is complement component C3.

Complement C3 is a protein of the complement pathway that is a convergence point for each of the three complement activation pathways (classical, lectin, and alternative), and functions as a regulator of the complement system terminal pathway. Upon innate immune system activation, C3 is cleaved into two substituent fragments, termed 3a and 3b. The C3a fragment is a 77 residue anaphylatoxin that binds to the C3a receptor (C3aR), and mediates a pro-inflammatory response, for example by causing mast cell degranulation of histamine.

The C3b fragment is a protein that is involved in several biological processes, for example marking pathogens for opsonization, and providing a feedback signal for amplifying the innate immune response.

Previous studies have shown that inhibition of Complement component C3 mediates protection from neuroinflammation. For example, in the context of traumatic brain injury, Boulos et al. J Neurosci. 2018 Aug. 15; 38(33): 7201-7203 describe that inhibition of C3 elicits a neuroprotective effect by preserving neuroreparatory mechanisms. In another example, Garragozloo et al. Acta Neuropathol. 2021 November; 142(5):899-915. doi: 10.1007/s00401-021-02366-4, describe that in a mouse model of EAE, inhibition of C3 mediates a neuroprotective effect by preventing retinal ganglion cell (RGC) loss and partially preserving neurites in the retina.

Although a role of C3 in neuroinflammation has been observed, the complement pathway plays a systemic role, which makes selective CNS modification challenging.

### Example 3: ASOs Targeting C3

This example describes design of RPMs (e.g., ASOs) that target human C3. In the context of diseases associated with neuroinflammation, it is desirable to decrease protein levels of C3 (e.g., by decreasing a level, transcription, splicing, and/or translation of C3 mRNA or by decreasing activity of C3 protein). In some embodiments, ASOs are designed to target regions of C3 mRNA that will result in decreased levels, transcription, splicing, and/or translation of C3 protein and/or decreased activity of C3 protein.

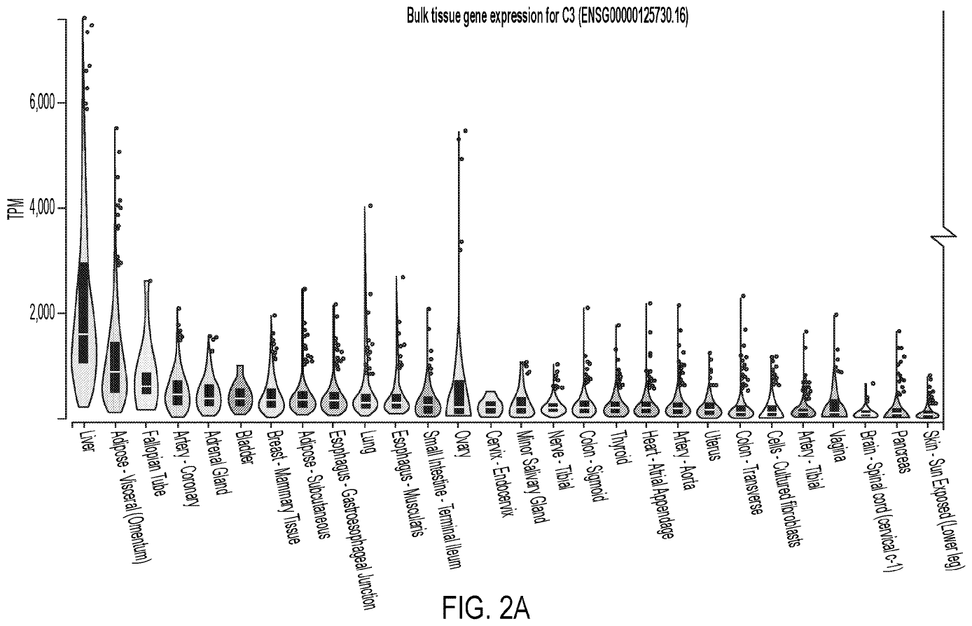

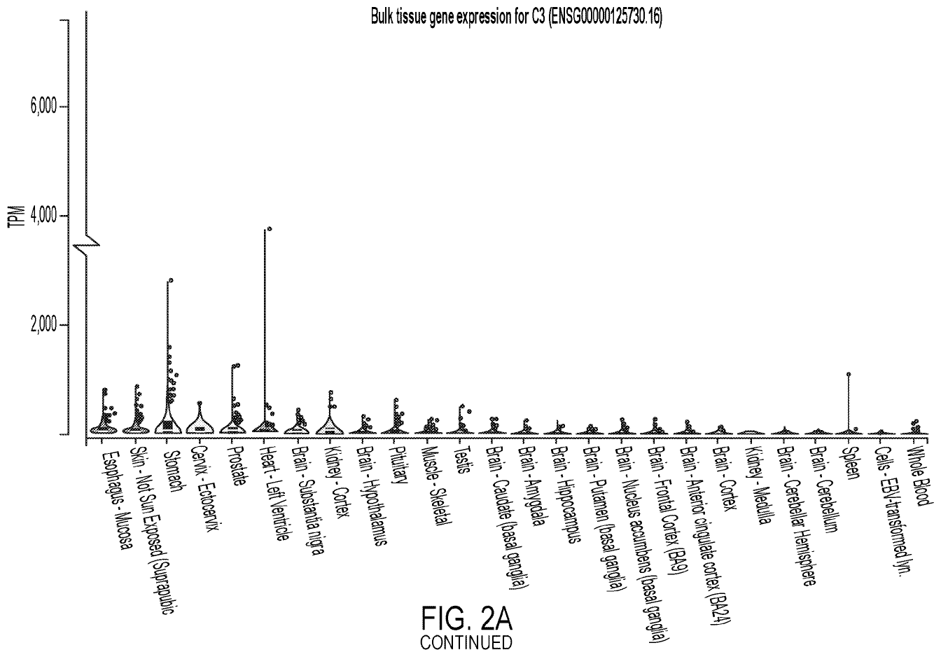

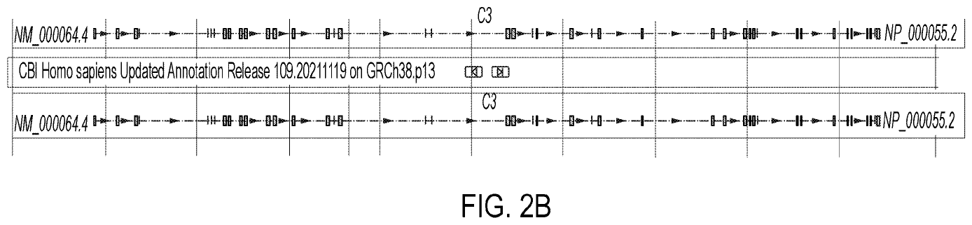

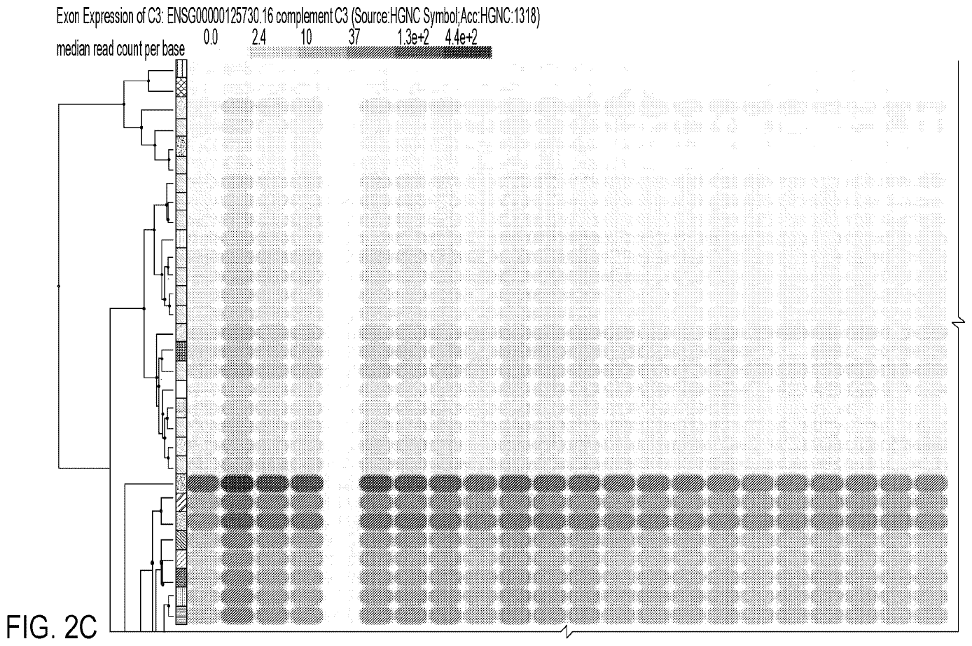

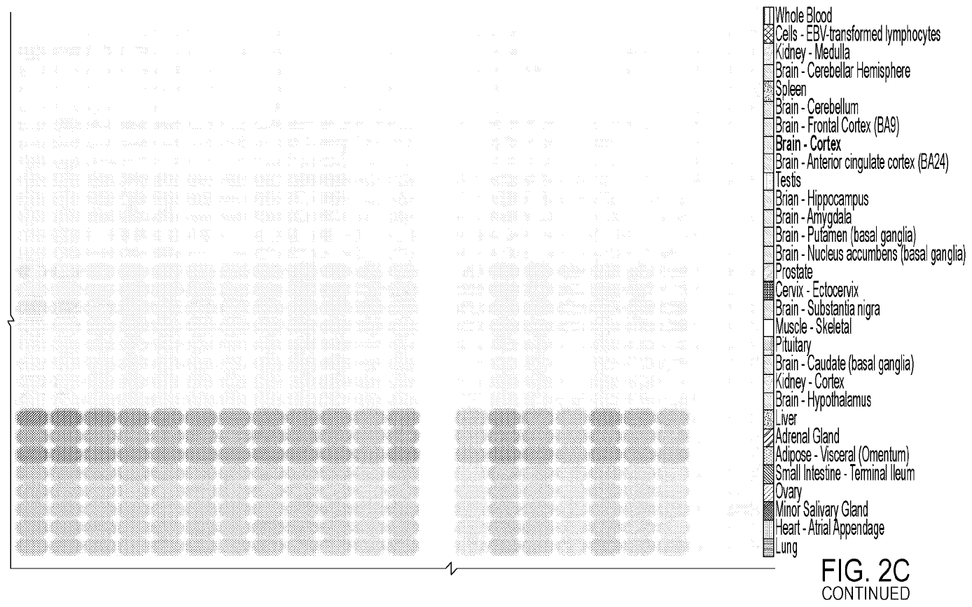

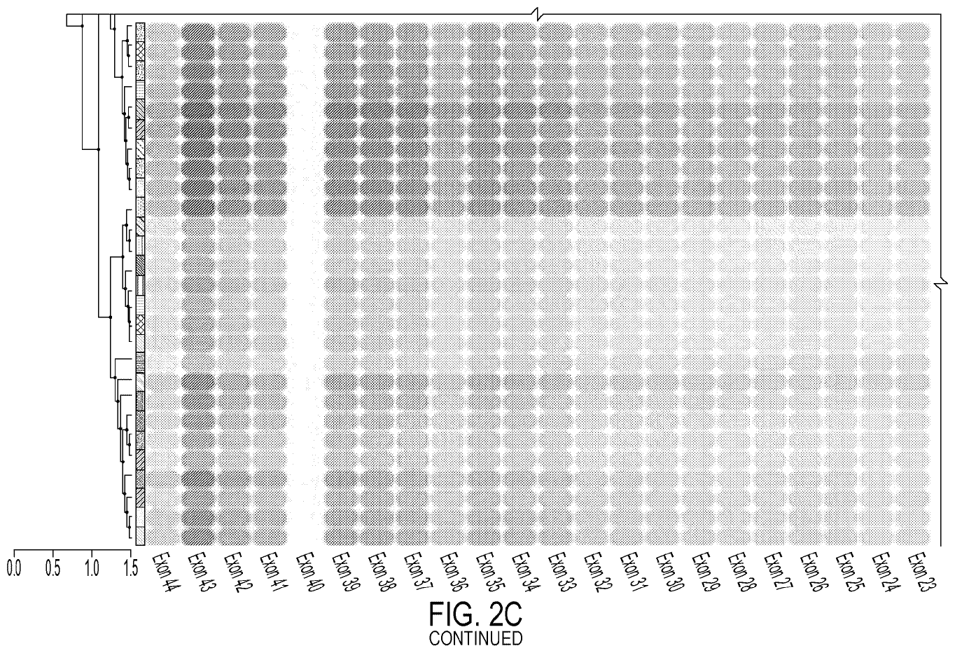

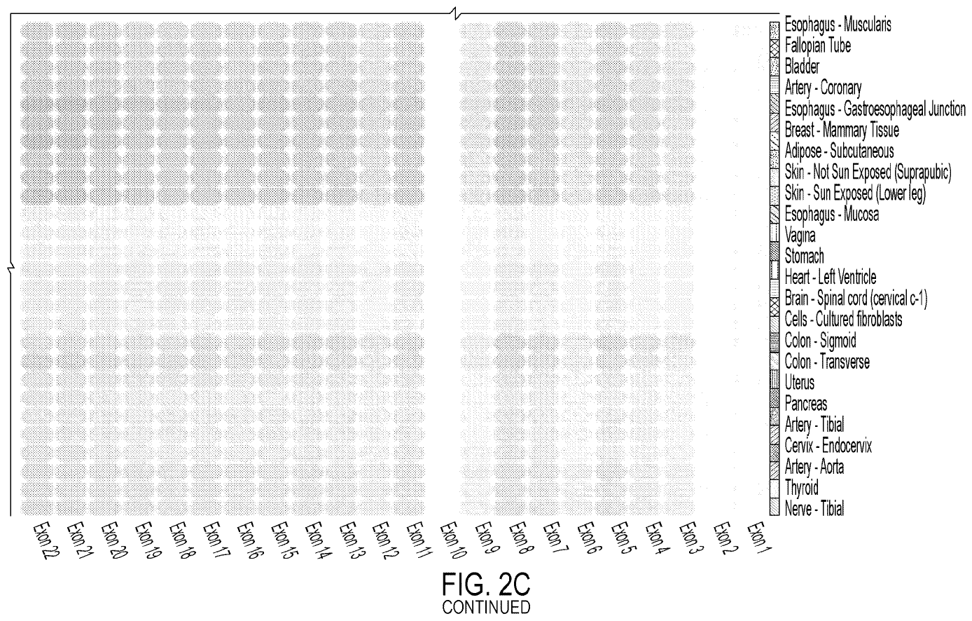

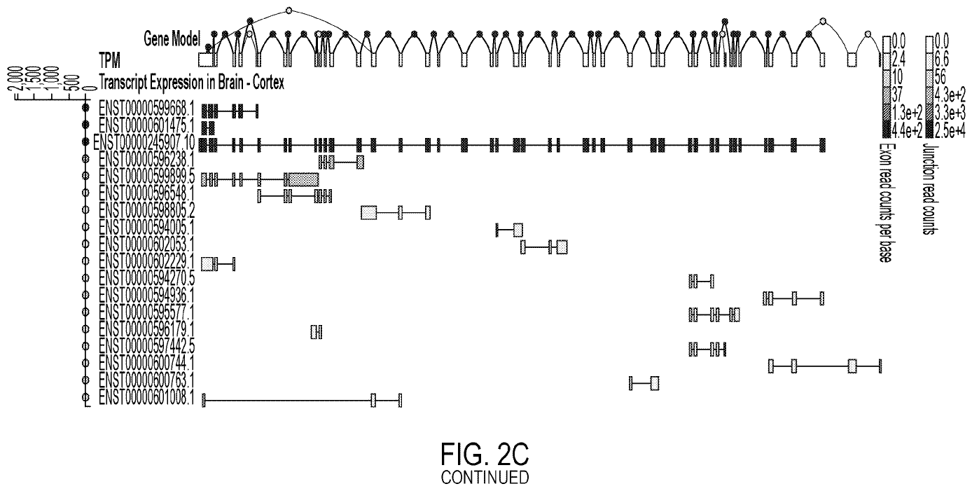

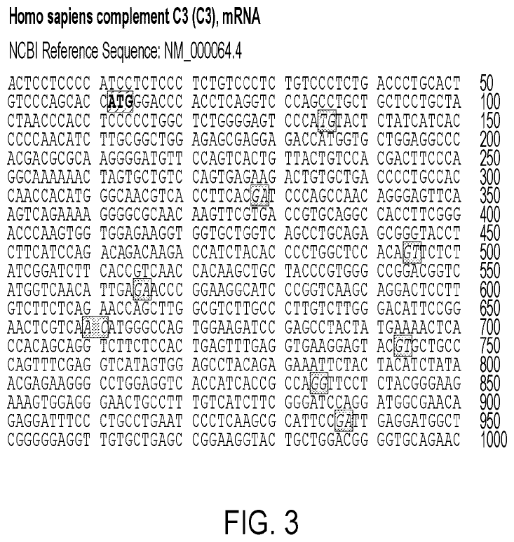

Expression profiling of C3 was performed. FIGS. 2A-2C show representative data regarding expression profiling of human complement component 3 (C3). FIG. 2A shows bulk tissue gene expression of human C3; data indicate C3 mRNA is ubiquitously expressed. FIG. 2B shows a schematic depicting exons and introns present in the C3 gene. FIG. 2C shows representative data for exon expression analysis of human C3 splice variants in tissue. FIG. 3 is a schematic depicting the primary AUG, and several exon-exon junctions of C3 mRNA transcript.

Embodiments of antisense oligonucleotides targeting human C3 complement mRNA are described in Table 1. In some embodiments, the ASOs comprise one or more chemical modifications and/or comprise a non-natural sugar-phosphate backbone (e.g., a phosphorothioate backbone). In some embodiments, the ASO has a “gapmer” structure.

### Example 4: In Vitro Screening of ASOs

Cell lines (e.g., U-188 MG cells) expressing human C3 are cultured and maintained using appropriate media (e.g., Dulbecco's Modified Eagle's Medium containing 10% fetal bovine serum). Cells carrying mutations in the C3 gene may also be used and are verified by sequencing. The assay is performed in 96 well plate format, seeding about 20,000 cells per well and treating with the ASOs at different concentrations of 5 nM and 20 nM using the Lipofectamine protocol. In parallel, cells treated with varying concentrations of C3 inhibitors optionally serve as controls for reduced C3 expression levels. Additionally, cells overexpressing C3 serve as controls for increased C3 expression levels. Cells are incubated at 37° C. in a cell culture incubator for 48 hours before isolating the total RNA. Total RNA is isolated and converted to cDNA, then Taqman gene expression assays are used to quantify the target (e.g., C3) gene modulation. Human housekeeping gene expression, such as HPRT1, is used to normalize the target transcript expressions.

Upon treatment of cells with ASOs, the levels of neuroinflammation markers profiled by methods well known in the art. For instance, immune activation products may be measured by a complement hemolytic activity assay. ASO efficacy is determined by comparing the levels of C3 mRNA levels, or C3 protein level or activity between treated and untreated cells.

To further characterize ASO-dependent changes in C3 function, cytotoxicity is measured to understand the physiological impact of changes in C3 transcript levels. Cell viability is measured by generating survival curves through manually counting Trypan blue staining of cells following ASO treatment. Alternatively, propidium iodide staining of cells followed by flow cytometry analysis is used to measure cell death.

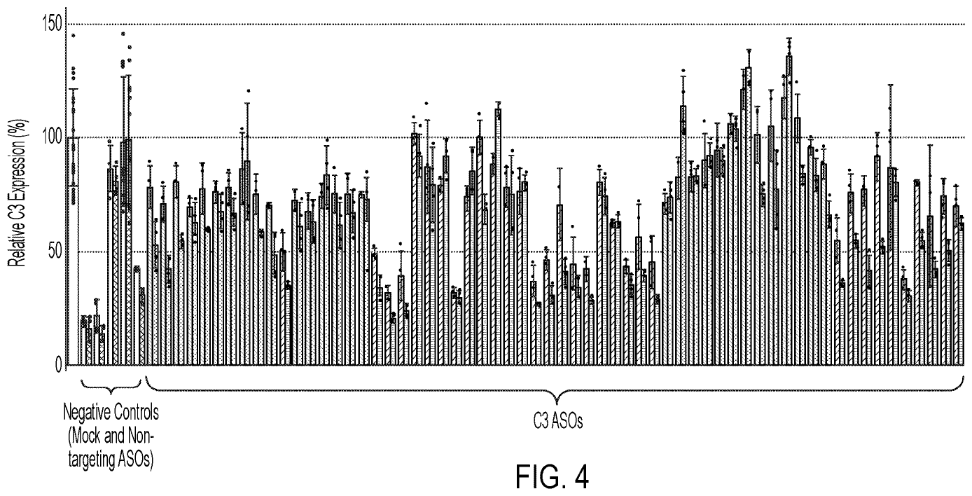

In one experiment, Hep3B cells were reverse-transfected using Lipofectamine 3000 with a panel of C3 ASOs (e.g., as set forth in Table 1) with 8 concentrations (20 and 5 nM). 48 h after transfection, RT-qPCR assay was performed to determine normalized expression levels of C3 mRNA (Taqman assay Hs00163811_m1, Thermofischer), using HPRT1 as a normalizer. FIG. 4 shows representative data for in vitro reduction of C3 mRNA in Hep3B cells in the 2-dose screening assay.

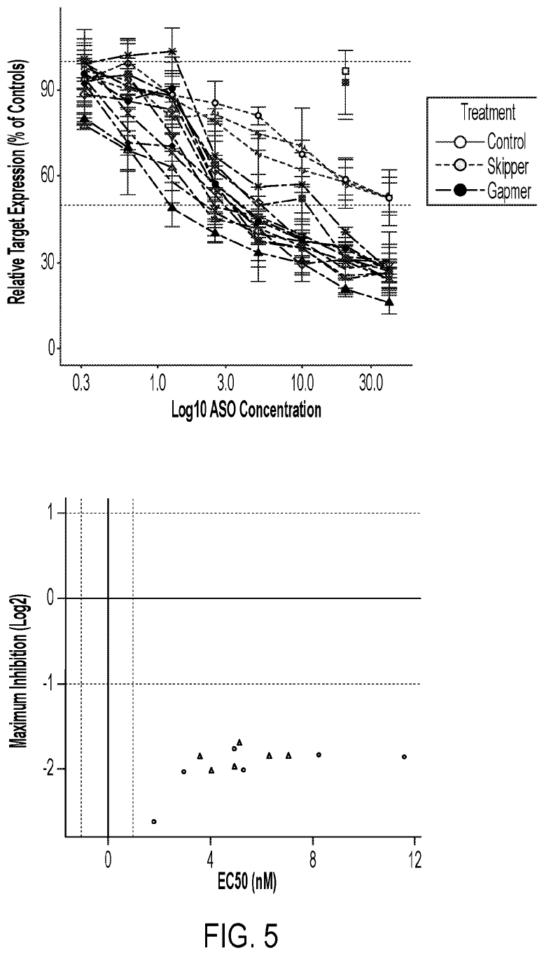

In another experiment, Hep3B cells were reverse-transfected using Lipofectamine 3000 with a panel of 16 ASOs identified in the 2-dose screen described above with 8 concentrations (40, 20, 10, 5, 2.5, 1.25, 0.625 and 0.3125 nM). 48 h after transfection, RT-qPCR assay was performed to determine normalized expression levels of C3 mRNA (Taqman assay Hs00163811_m1, Thermofischer), using HPRT1 as a normalizer. FIG. 5 shows representative data for representative data for in vitro reduction of C3 mRNA in Hep3B cells. The top panel shows C3 mRNA levels presented relative to mock-transfected controls in function of the dose of transfected ASOs for the 16 tested C3 ASOs (Skippers and Gapmers shown; means are presented, error bars are Standard Error for N=2 biological replicates by group. The bottom panel shows maximum inhibition (log 2, Y-axis) plotted in function of the observed EC50 (X-axis); the most potent ASOs are in the lower left part of the dot-plot.

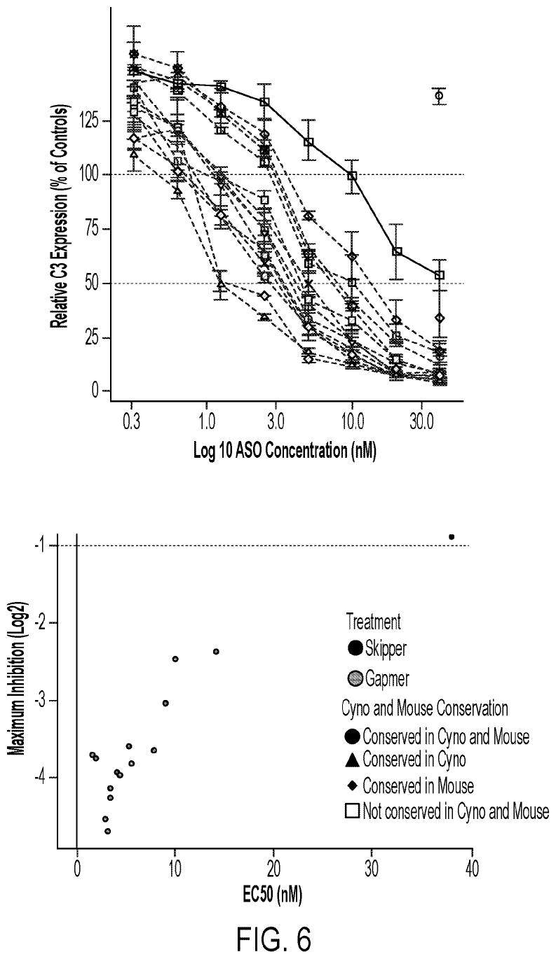

A similar experiment was also performed in Hep2G cells. The HepG2 cells were reverse-transfected using Lipofectamine 2000 with a panel of 9 ASOs (corresponding to derivatives of SEQ ID NO: 16) with 8 concentrations (40, 20, 10, 5, 2.5, 1.25, 0.625 and 0.3125 nM). 48 h after transfection, RT-qPCR assay was performed to determine normalized expression levels of C3 mRNA (Tagman assay Hs00163811_m1, Thermofischer), using HPRT1 as a normalizer. FIG. 6 shows representative data for representative data for in vitro reduction of C3 mRNA in Hep2G cells. The top panel shows C3 mRNA levels presented relative to mock-transfected controls in function of the dose of transfected ASOs for the tested C3 ASOs (Skippers in dark shading, Gapmers in light shading); means are presented, error bars are Standard Error for N=2 biological replicates by group The bottom panel shows maximum inhibition (log 2, Y-axis) plotted in function of the observed EC50 (X-axis); the most potent ASOs are in the lower left part of the dot-plot.

### Example 5: In Vivo ASO Methods

An animal model of experimental autoimmune encephalomyelitis (EAE), for example as described by Michailidou et al. Acta Neuropathologica Communications (2018) 6:36, is used as an indicator of C3 levels or activity or function. ASOs targeting C3 are administered to the animals by infusion. When multiple ASO infusions are performed, administration of the ASO is done at the same time each day to minimize changes in metabolism due to circadian rhythm. ASO infusions are either directly provided to the affected area or into the cerebral spinal fluid (CSF). Animals may be placed in the Trendelenburg position during and after the infusion to aid in distribution of the ASOs into the tissue (e.g., CNS tissue) of the animals. ASOs are solubilized in an appropriate buffer and sterilized prior to infusions. Following infusions, animals are maintained for a predetermined period of time prior to analysis. Harvested tissue samples are then assessed for EAE-induced damage by histology. Blood samples are isolated and serum levels of C3 are measured from experimental and control animals.

### Example 6: In Vivo Knockdown of C3 mRNA in Mouse Subjects

This example describes in vivo knockdown of C3 mRNA using ASOs described herein. Briefly, mice were administered once-weekly ICV injections (3 doses total) of the ASOs, and mRNA levels were quantified in the brain tissue (samples harvested from Left Cortex 1 and Left Hippocampus). Frozen tissues were lysed and homogenized in RLT buffer using beads (MP Biomedical) before RNA extraction using RNeasy Mini Kit (Qiagen) in a QIAcube station (Qiagen). RNA concentration was evaluated a nanodrop spectrometer (ThermoFisher), integrity using 2100 Bioanalyzer LabChip (Agilent). 500 ng of RNA was reverse-transcribed using SuperScript IV VILO Master Mix ezDNase (Invitrogen). qPCR was performed with TaqMan Fast Advance Master Mix (Invitrogen) in a Quantstudio thermocycler (Applied Biosystems) with 2 independent Taqman (VIC) assays for C3 (IDT TF TF Mm01232778_m1, TF Mm01232785_m1). Pgkl and PPIA levels were measured using a Tagman (FAM) assay (Mm00435617_m1, Thermofischer) for normalization using DeltaDeltaCt method.

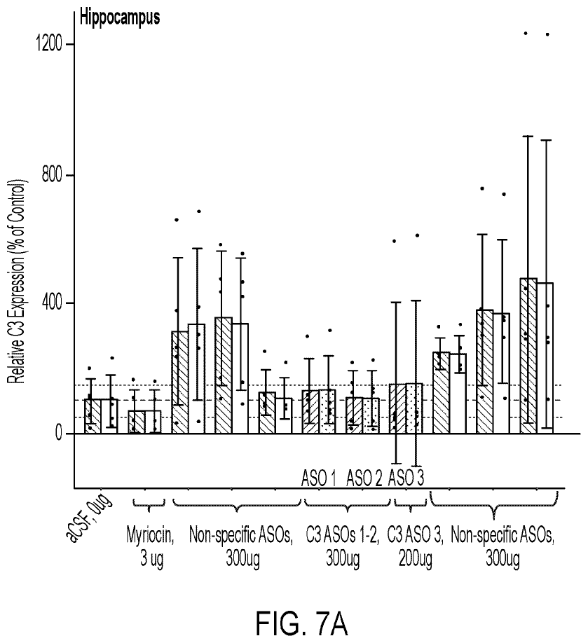

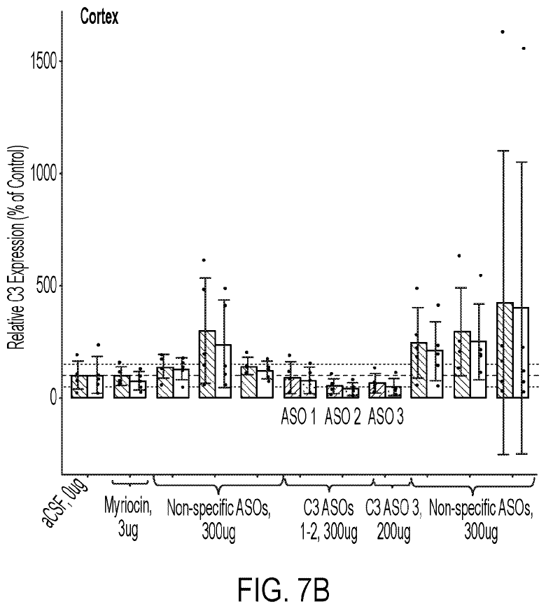

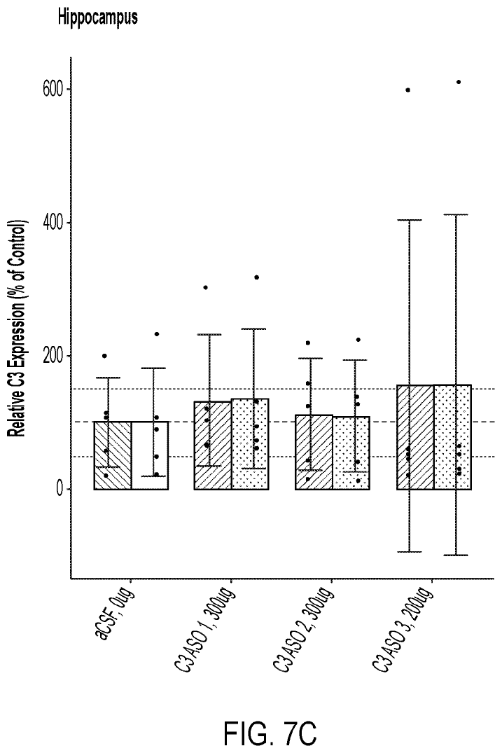

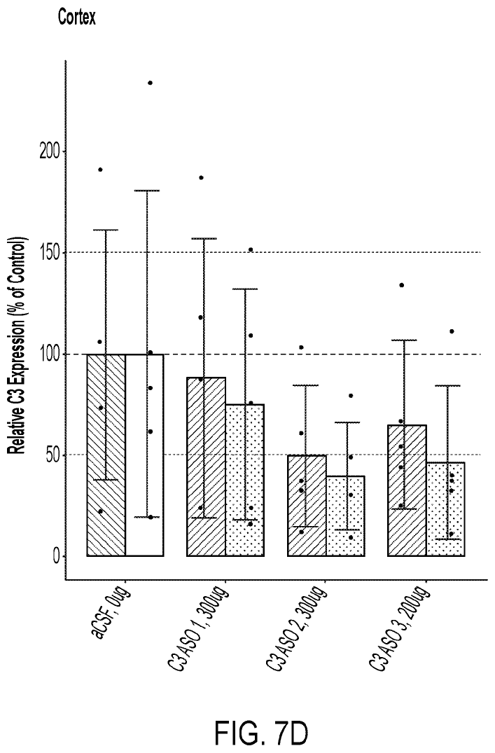

FIGS. 7A-7D show representative data for in vivo reduction of C3 mRNA levels in mouse brain. FIG. 7A shows relative C3 mRNA levels in hippocampus tissues of mouse subjects seven days after the last dose of a three-dose (e.g., 1 dose per week) series of ICV injections of vehicle (artificial CSF), 3 ug (total) of myriocin, a non-C3-specific ASO at a total dose of 300 ug (100 ug+100 ug+100 ug), C3 ASOs 1 and 2, each at a total dose of 300 ug (100 ug+100 ug+100 ug), or C3 ASO 3 at a total dose of 200 ug (100 ug+50 ug+50 ug). FIG. 7B shows relative C3 mRNA levels in cortex tissues of mouse subjects seven days after the last dose of a three-dose (e.g., 1 dose per week) series of ICV injections of vehicle (artificial CSF), 3 ug (total) of myriocin, a non-C3-specific ASO at a total dose of 300 ug (100 ug+100 ug+100 ug), C3 ASOs 1 and 2, each at a total dose of 300 ug (100 ug+100 ug+100 ug), or C3 ASO 3 at a total dose of 200 ug (100 ug+50 ug+50 ug). FIG. 7C shows relative C3 mRNA levels in hippocampus tissues of mouse subjects one week after the last of three ICV injections through a canula, with 1 week interval, of vehicle (artificial CSF), 100 ug+100 ug+100 ug of C3 ASO 1 or C3 ASO 2, or 100 ug+50 ug+50 ug of C3 ASO 3. FIG. 7D shows relative C3 mRNA levels in cortex tissues of mouse subjects one week after the last of three ICV injections through a canula, with 1 week interval, of vehicle (artificial CSF), 100 ug+100 ug+100 ug of C3 ASO 1 or C3 ASO 2, or 100 ug+50 ug+50 ug of ASO 3. “C3 ASO 1” comprises the nucleotide sequence of SEQ ID NO: 16, a gapmer structure, and the chemical modifications as set forth in Columns A and C of row 17 of Table 1. “C3 ASO 2” comprises the nucleotide sequence of SEQ ID NO: 156, a gapmer structure, and the chemical modifications as set forth in Columns A and C of row 157 of Table 1. “C3 ASO 3” comprises the nucleotide sequence of SEQ ID NO: 71, a gapmer structure, and the chemical modifications as set forth in Columns A and C of row 72 of Table 1.

In the repeated ICV injection study, some animals exhibited minor acute in-life observations post administration of myriocin, non-targeting ASO, or C3 ASOs 1-3, which were largely resolved in 1-2 hours. No major effects on life were observed in animals that received administration of C3 ASOs 1 or 3.

Repeated ICV injection of C3 ASOs 1, 2, and 3 resulted in C3 mRNA knockdown by 12-25%, 51-60%, and 35-53%, respectively, in the cortex (FIGS. 7B and 7D). No significant effects on C3 mRNA were observed in subjects that were administered control treatments of vehicle, myriocin, or non-specific ASOs.

### Example 7: ASO Administration To an In Vivo Model of Age-Related Macular Degeneration

This Example relates to the pharmacodynamic and functional effects of C3 ASOs on an in vivo mouse model of age-related macular degeneration (AMD).

Thirty-two female C57BL/6J mouse subjects at 8-10 weeks of age underwent a 21-day analysis. Following an ocular examination to obtain baseline readings on day 0, mouse subjects were split into four treatment groups (8 subjects each; N=16 eyes per group). The negative control group received intravitreal (IVT) (temporal) oculus uterque (OU) injection of vehicle on day 0. The positive control group received no treatment until day 7 when they were administered a 40 μg dose of Aflibercept, an AMD agent, via IVT OU injection. One C3 ASO treatment group received a single 50 μg dose of an ASO comprising the nucleotide sequence of SEQ ID NO: 156, a gapmer structure, and the chemical modifications as set forth in Columns A and C of row 157 of Table 1 (alternatively referred to as “C3 ASO 2” herein) via IVT (temporal) OU injection on day 0. A separate C3 ASO treatment group received a first 50 μg dose of an ASO comprising the nucleotide sequence of SEQ ID NO: 156, a gapmer structure, and the chemical modifications as set forth in Columns A and C of row 157 of Table 1 (alternatively referred to as “C3 ASO 2” herein) via IVT (temporal) OU injection on day 0 and a second 50 μg dose of the same ASO via IVT (nasal) OU injection on day 7 (total dose 100 μg).

Subject body weight was monitored weekly and subject morbidity was monitored daily during the 21-day period. All mouse subjects underwent laser-induced choroidal neovascularization (CNV) at day 14. To assess CNV lesions, fluorescein angiography imaging analyses were performed on all subjects at day 21 prior to being sacrificed. CNV lesions were also assessed by immunohistochemistry (IHC) analyses of choroid/retinal pigment epithelium (RPE) flat mounts which were prepared after sacrificing mouse subjects. IHC staining conditions comprised phalloidin, isolectin, C3, C5b-9, and CD68. The therapeutic endpoints analyzed included CNV formation, vascular leakage, safety (in-life monitoring), and target engagement.

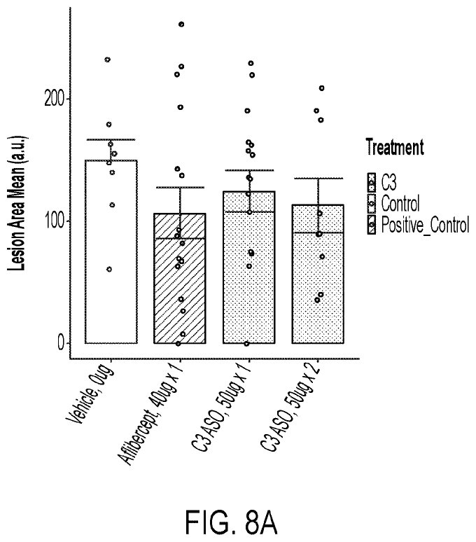

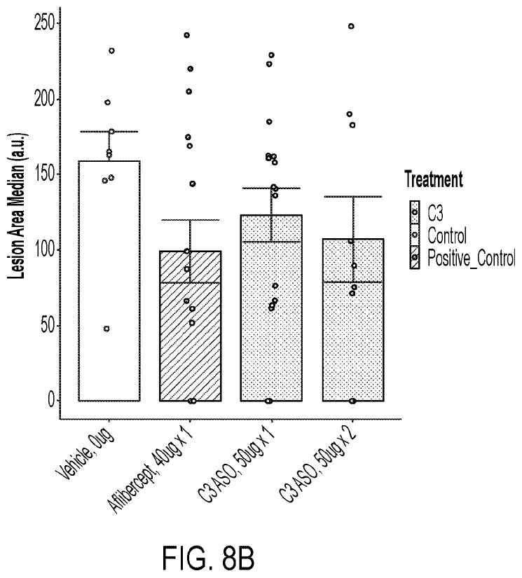

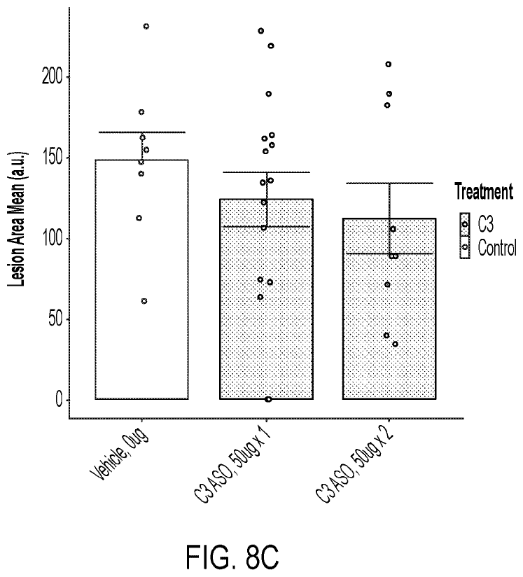

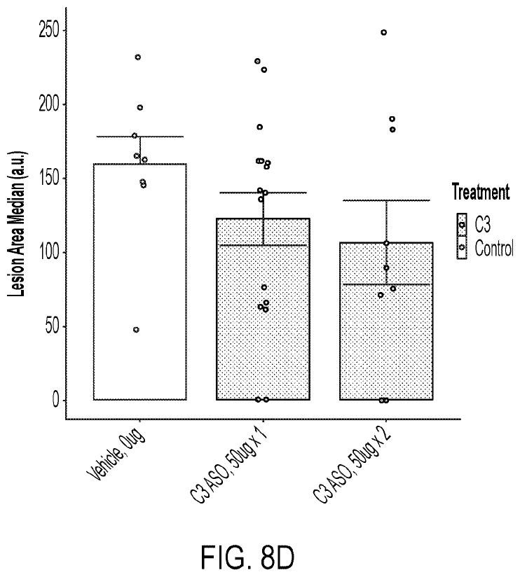

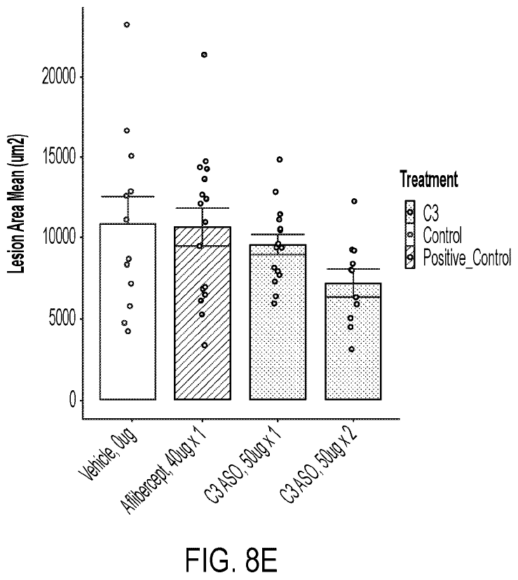

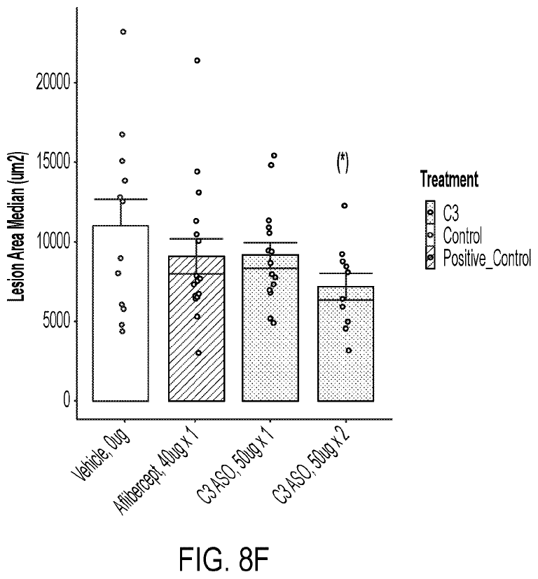

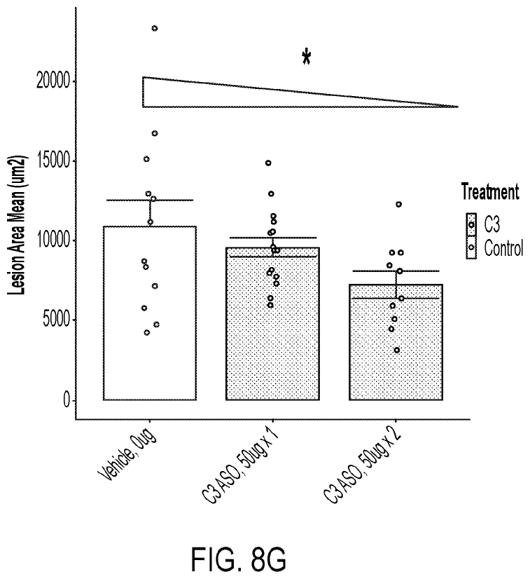

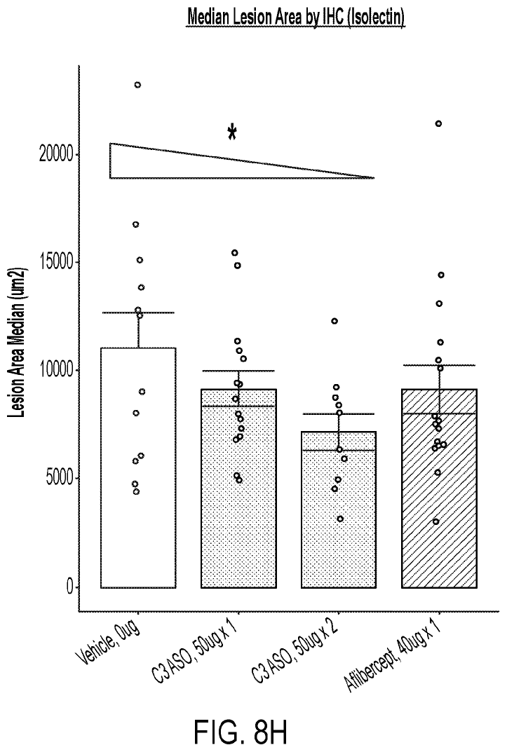

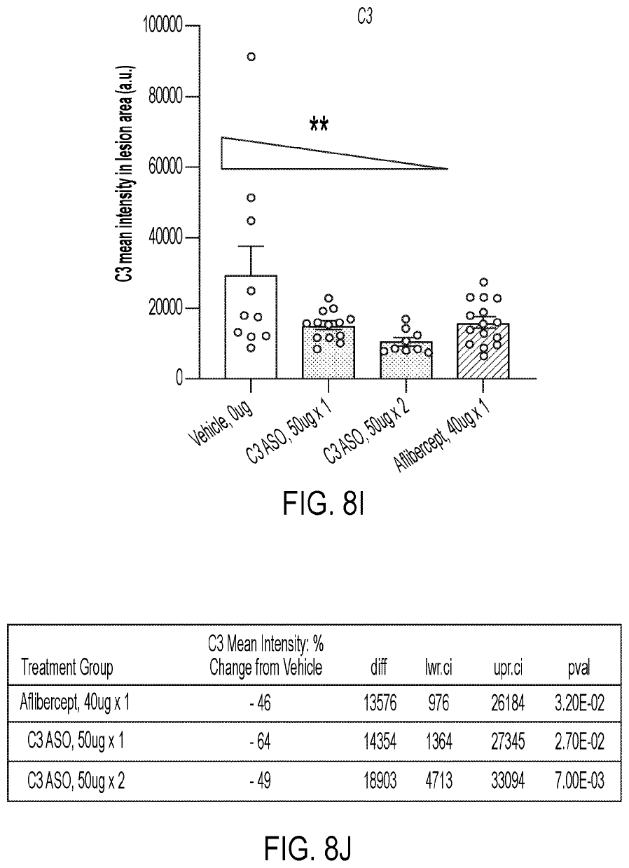

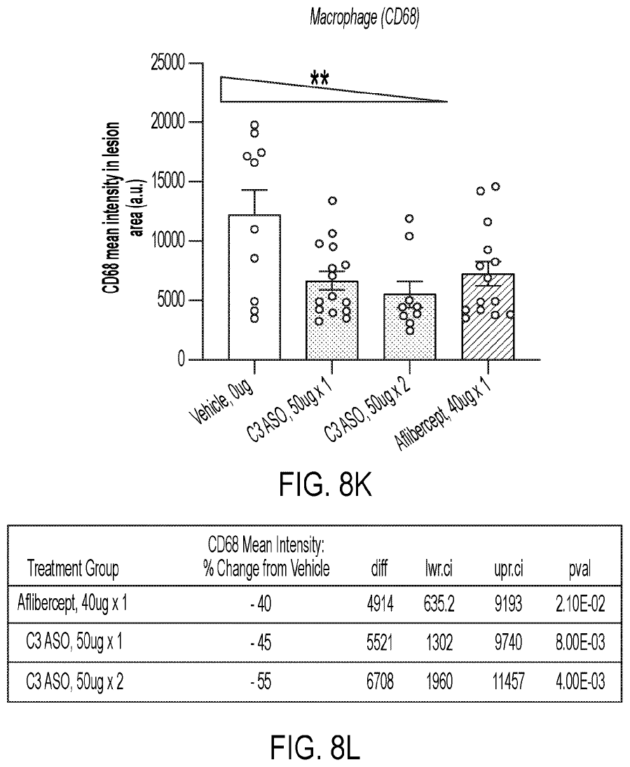

Results from fluorescein angiography analyses are shown in FIGS. 8A-8D. Mean lesion area was reduced by 28% and median lesion area was reduced by 38% in subjects that received IVT injection of Aflibercept relative to vehicle-treated subjects. Mean lesion area was reduced by 17% and median lesion area was reduced by 23% in subjects that received single IVT injection of C3 ASO relative to vehicle-treated subjects. Mean lesion area was reduced by 24% and median lesion area was reduced by 33% in subjects that received repeated IVT injection of C3 ASO relative to vehicle-treated subjects (FIGS. 8A-8D).

Results from IHC analyses of isolectin staining of RPE flat mounts are shown in FIGS. 8E-8L. Mean lesion area was reduced by 2% and median lesion area was reduced by 17% in subjects that received IVT injection of Aflibercept relative to vehicle-treated subjects. Mean lesion area was reduced by 12% and median lesion area was reduced by 17% reduction in subjects that received single IVT injection of C3 ASO relative to vehicle-treated subjects. Mean lesion area was reduced by 34% and median lesion area was reduced by 35% in subjects that received repeated IVT injection of C3 ASO relative to vehicle-treated subjects (FIGS. 8E-8H).

Statistical analyses of isolectin staining showed repeated IVT injection of C3 ASO resulted in a statistically significant decrease in mean lesion area and medial lesion area (FIGS. 8F-8H). A reduction in protein levels of C3 and CD68, a marker of macrophages/inflammation, within the CNV lesion area was also observed. Reductions in C3 and CD68 were dependent on the dose of C3 ASO that was administered (FIGS. 8I-8L). These results indicated a dose-dependent effect of C3 ASO administration on CNV-induced lesions in mouse eyes.

### Example 8: Immunostimulatory Effects of ASOs In Vitro

This Example describes analyses of immunostimulatory effects of C3 ASOs in human peripheral blood mononuclear cells (huPBMCs).

All ASOs were prepared using in vivo quality grade material in manner that was consistent with analyses performed in animal subjects as described above. huPBMCs were harvested from healthy donors and went either untreated, or were treated with a cytokine/chemokine response control agent, or treated with C3 ASO 2 (comprising the nucleotide sequence of SEQ ID NO: 156, a gapmer structure, and the chemical modifications as set forth in Columns A and C of row 157 of Table 1) at a concentration of 1 μM, 3 μM, or 10 μM for 24 hours.

Cytokine/chemokine response control agents included: XD-01024, a cholesterol-conjugated ApoB siRNA which has TLR7/8 agonist effects; CL097, a water-soluble derivative of imidazoquinoline compound R848 which is a TLR7/8 ligand; Imiquimod (R837), an immune response modifier having potent antiviral activity and also induces production of cytokines and activates TLR7; ODN2216 a 20mer oligo containing unmethylated CpG and has TLR9 agonist effects; ODN2006 having preference towards TLR9, one or more CpGs, class B; ODN2395, TLR9, CpG with palindromic motif, class C; TL8-506, a benzoazepine compound which has TLR8 agonist effects; LMW poly(l:c) which has TLR3 agonist effects; and XD-00366, a 25mer double-stranded, unmodified, blunt-ended LacZ RNA duplex which has TLR7/8 agonist effects.

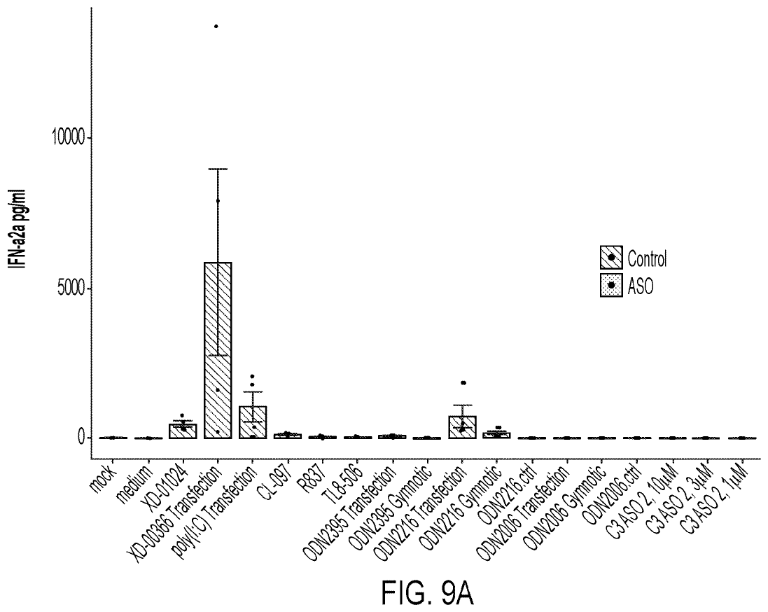

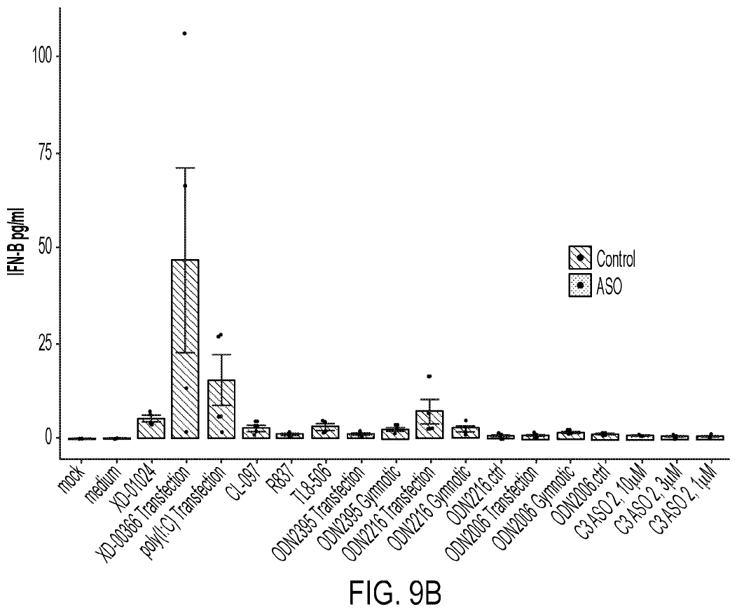

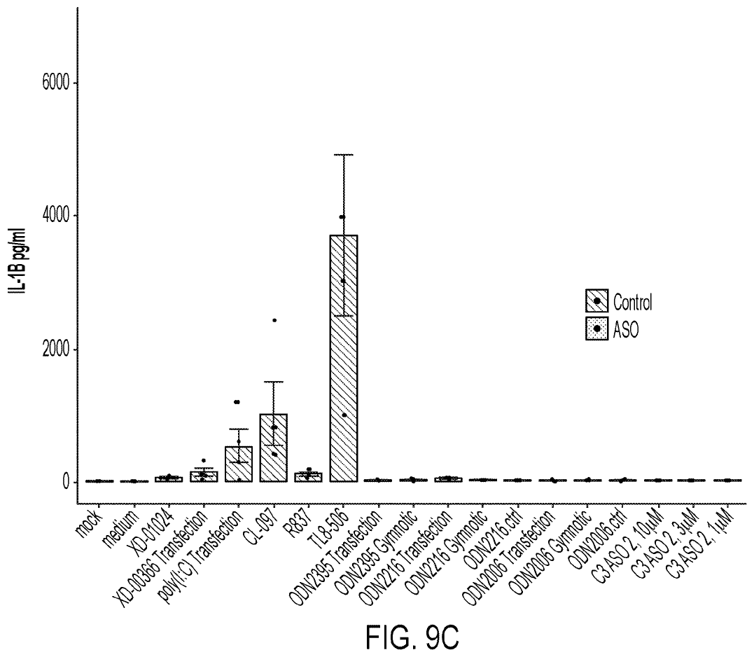

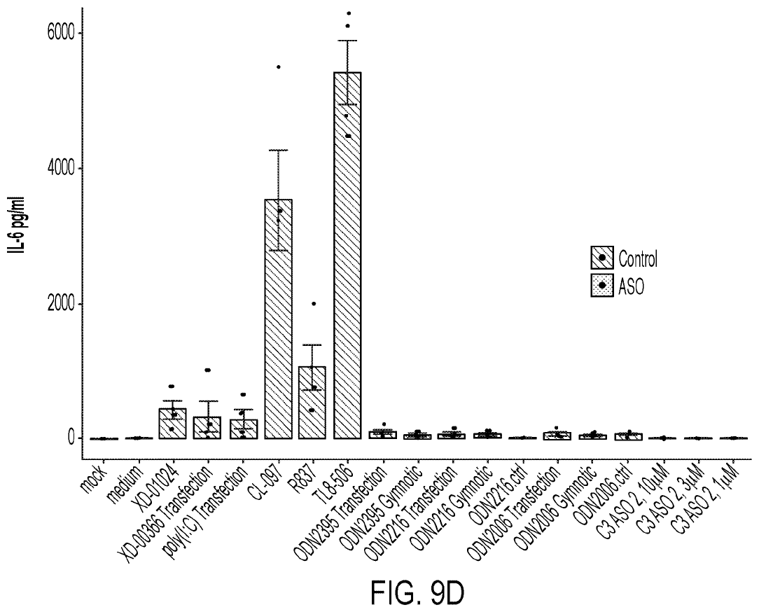

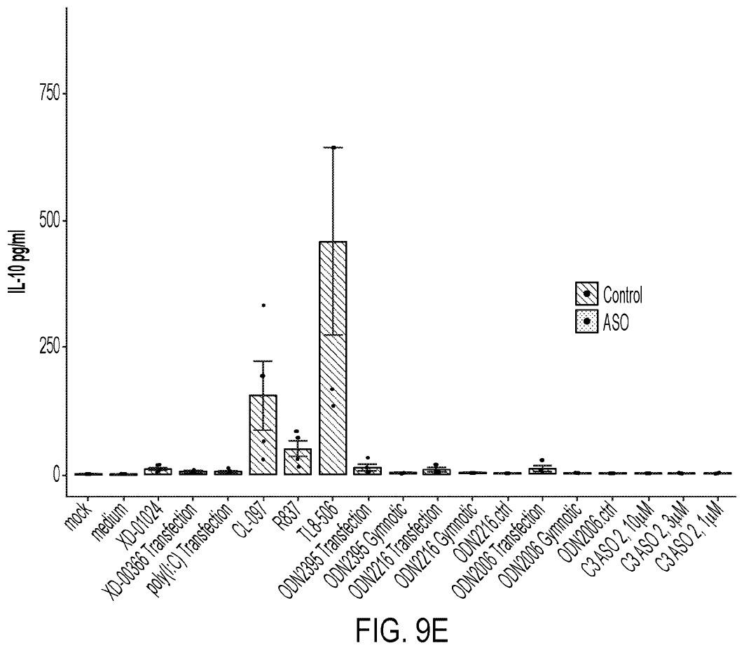

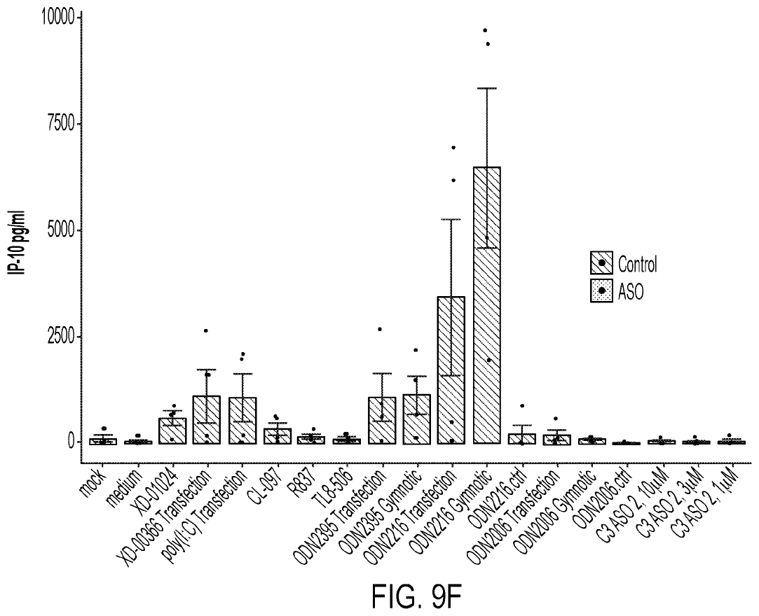

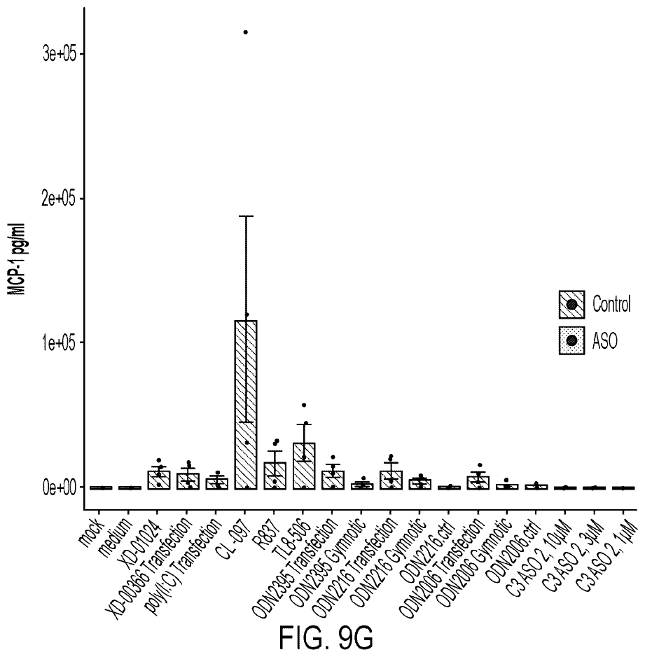

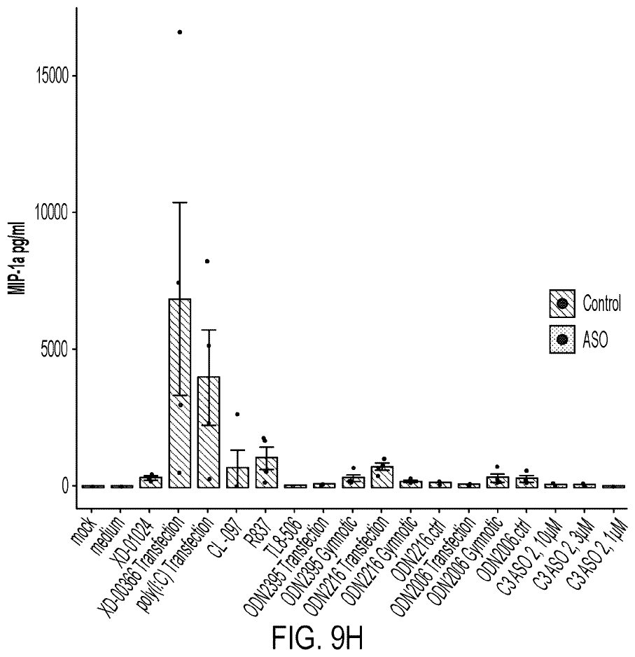

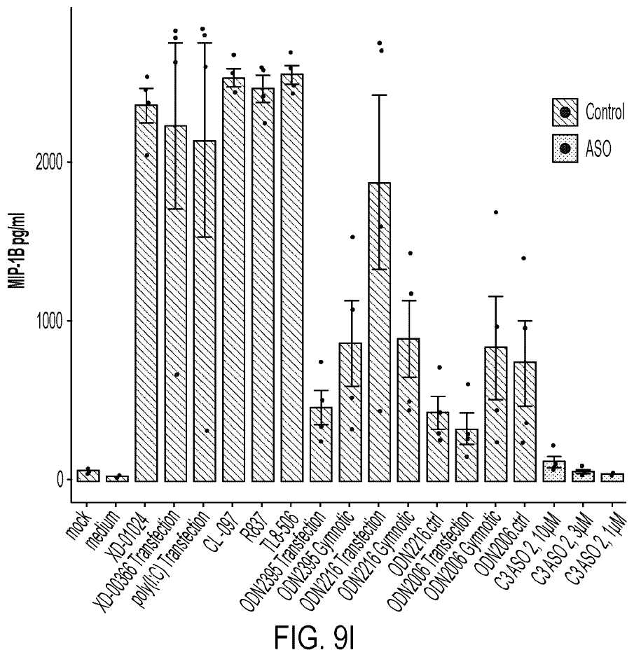

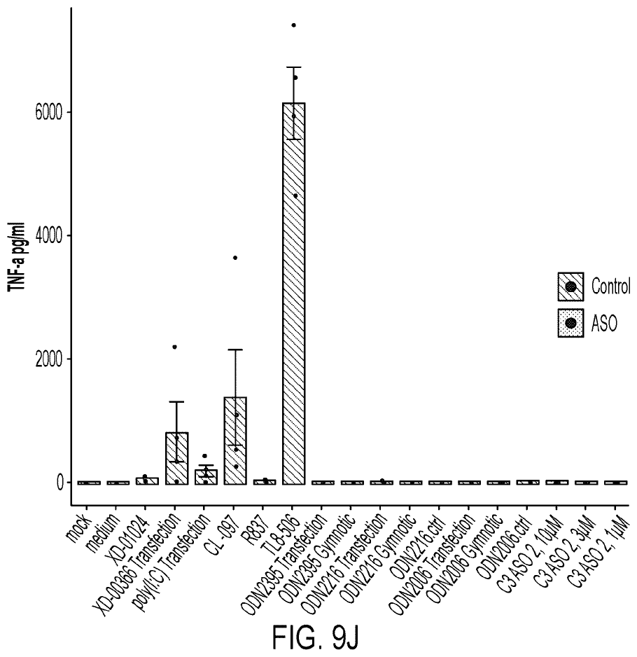

Following treatment of huPBMCs, the levels of IFN-a2a, IFN-b, IL-1B, IL-6, IL-10, IP-10, MCP-1, MIP-1a, MIP-1b and TNF-a were measured using the MSD-U-Plex platform. Representative data from these analyses are shown in FIGS. 9A-9J. Negative control cells exhibited minimal increases or no detectable increase in chemokine/cytokine levels following treatment. Treatment with TLR agonist positive controls resulted in increased chemokine/cytokine levels as expected. When compared relative to cell samples treated under negative or positive control conditions, no immunogenic responses to C3 ASO 2 detected (FIGS. 9A-9J).

### Example 9: In Vivo ASO Administration to Non-Human Primates

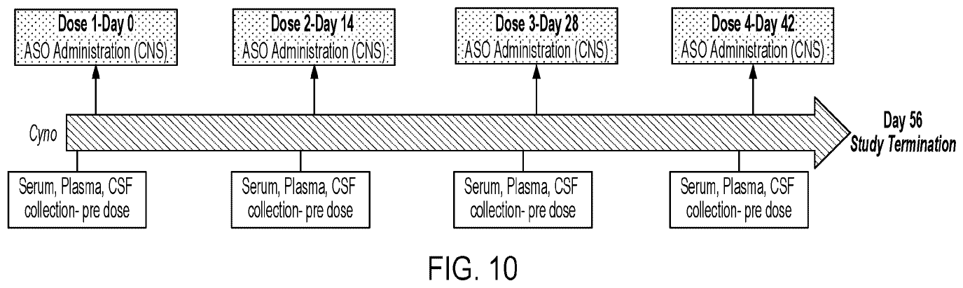

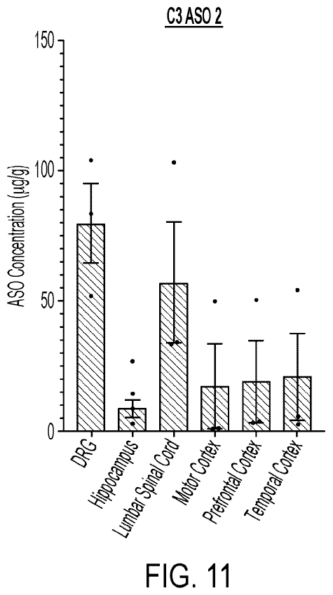

This example describes in vivo administration of C3 ASOs to cynomolgus monkey (Macaca fascicularis) subjects (also referred to as “non-human primate subjects”) and analyses of in vivo pharmacokinetics (ASO levels). Briefly, three male non-human primate subjects were administered a series of four intrathecal (“IT”) injections of either vehicle (artificial CSF) or ASO (“C3 ASO 2” which comprises the nucleotide sequence of SEQ ID NO: 156, a gapmer structure, and the chemical modifications as set forth in Columns A and C of row 157 of Table 1) at a dose of 80 mg (20 mg+20 mg+20 mg+20 mg). For all non-human primate subjects, each round of IT injection was performed two weeks apart (days 0, 14, 28, and 42). Samples of cerebrospinal fluid as well as brain (frontal cortex, sensory cortex, and hippocampus), lumbar spinal cord, dorsal root ganglion, kidney, liver, spleen, heart, stomach, and gonads tissues were collected at two weeks following the last IT injection (FIG. 10). Liquid chromatography-tandem mass spectrometry (LC-MS/MS) analysis was used to measure ASO pharmacokinetics (ASO levels) in tissue samples obtained from injected non-human primates. ASO concentrations in tissue samples were quantified to determine ASO levels as a result of IT injection (FIG. 11).

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. An isolated nucleic acid that comprises a region of complementarity with a human C3 mRNA transcript, a nucleotide sequence that is at least 60% identical to any one of the nucleotide sequences set forth in SEQ ID NOs: 1-210, and upon binding to the mRNA transcript decreases transcription, splicing, and/or translation of functional C3 protein encoded by the mRNA transcript.

2. The isolated nucleic acid of claim 1, wherein the isolated nucleic acid comprises RNA.

3. The isolated nucleic acid of claim 1 or 2, wherein the isolated nucleic acid is an antisense oligonucleotide.

4. The isolated nucleic acid of any one of claims 1 to 3, comprising or consisting of between and 40 nucleotides.

5. The isolated nucleic acid of claim 4, wherein the isolated nucleic acid comprises or consists of between 18 and 25 nucleotides.

6. The isolated nucleic acid of any one of claims 1 to 5, wherein the isolated nucleic acid comprises one or more chemical modifications.

7. The isolated nucleic acid of claim 6, wherein the one or more chemical modifications comprise one or more nucleoside modifications and/or one or more sugar-phosphate backbone modifications.

8. The isolated nucleic acid of claim 7, wherein the one or more nucleoside modifications comprises a 2′-O-methyl (2′-OMe) modification, a 2′-O-MOE modification, a 2′-O-fluoro modification, or a locked nucleic acid (LNA) modification.

9. The isolated nucleic acid of claim 7 or 8, wherein the one or more sugar-phosphate backbone modifications comprises a phosphorothioate backbone modification.

10. The isolated nucleic acid of any one of claims 1 to 9, wherein the isolated nucleic acid is fully chemically modified.

11. The isolated nucleic acid of any one of claims 1 to 10, wherein the isolated nucleic acid comprises one or more deoxyribonucleotides, optionally wherein the isolated nucleic acid is a gapmer.

12. The isolated nucleic acid of any one of claims 1 to 11, wherein the region of complementarity is located in an untranslated region (UTR) of the C3 mRNA transcript.

13. The isolated nucleic acid of claim 12, wherein the untranslated region comprises a 5′ UTR, an intron, or a 3′ UTR of the C3 mRNA transcript.

14. The isolated nucleic acid of any one of claims 1 to 11, wherein the region of complementarity is located in a protein coding region of the C3 mRNA transcript.

15. The isolated nucleic acid of any one of claims 1 to 11, wherein the region of complementarity is located on an intron-exon boundary of the C3 mRNA transcript.

16. The isolated nucleic acid of any one of claims 1 to 15, wherein the region of complementarity comprises at least 10, 11, 12, 13, 14, 15, 16, 17, 18, 19, or 20 continuous nucleotides of the sequence set forth in SEQ ID NO: 211.

17. The isolated nucleic acid of any one of claims 1 to 16, comprising the nucleotide sequence set forth in any one of the nucleotide sequences set forth in Table 1.

18. A composition comprising the isolated nucleic acid of any one of claims 1 to 19, and a pharmaceutically acceptable excipient.

19. A method for decreasing a level, transcription, splicing, and/or translation of Complement component C3 in a cell or subject, the method comprising administering the isolated nucleic acid of any one of claims 1 to 17 or the composition of claim 18 to a cell or a subject in need thereof.

20. The method of claim 19, wherein the subject is characterized as having neuroinflammation.

21. The method of claim 19 or 20, wherein the subject comprises one or more mutations in a gene that is associated with neuroinflammation, optionally wherein the gene is C3.

22. The method of any one of claims 19 to 21, wherein the cell is a human cell, optionally wherein the cell is in a subject.

23. The method of any one of claims 19 to 22, wherein the subject is a human subject.

24. The method of any one of claims 19 to 23, wherein the subject has or is suspected of having a disease or disorder associated with neuroinflammation, optionally wherein the neuroinflammation is iatrogenic neuroinflammation.

25. The method of claim 24, wherein the disease or disorder is dry age-related macular degeneration (dry AMD) (e.g., dry AMD with geographic atrophy), multiple sclerosis (MS), peripheral neuropathies (e.g., Guillain Barr syndrome (GBS), Chronic inflammatory demyelinating polyneuropathy (CIDP), etc.), neuromyelitis optica (NO), myasthenia gravis (MG), Alzheimer's disease, frontotemporal dementia (FTD), iatrogenic neuroinflammation (e.g., inflammation associated with adeno-associated virus administration, ASO administration, antibody administration (such as Amyloid-Related Imaging Abnormalities (ARIA), for example, Amyloid-Related Imaging Abnormalities edema (ARIA-E) neuroinflammation), etc.), and acute neuronal injury (e.g., traumatic brain injury (TBI), spinal cord injury, stroke, etc.).

26. The method of any one of claims 19 to 25, wherein the administration is systemic administration, optionally wherein the systemic administration comprises intravenous injection.

27. The method of any one of claims 19 to 25, wherein the administration comprises direct administration to a target tissue of the subject, optionally wherein the direct administration comprises direct injection to the central nervous system (CNS), direct injection to the peripheral nervous system, or direct administration to the eye.

28. The method of claim 27, wherein the administration comprises placing the subject in a Trendelenburg position during the administration.

29. A method for decreasing serum C3 levels in a subject, the method comprising administering the isolated nucleic acid of any one of claims 1 to 17, or the composition of claim 18, to a subject in need thereof.

30. The method of claim 29, wherein the subject is characterized as having neuroinflammation.

31. The method of claim 29 or 30, wherein the subject comprises one or more mutations in a gene that is associated with neuroinflammation, optionally wherein the gene is C3.

32. The method of any one of claims 29 to 31, wherein the cell or subject is a human cell or subject.

33. The method of any one of claims 29 to 32, wherein the subject has or is suspected of having a disease or disorder associated with neuroinflammation.

34. The method of claim 33, wherein the disease or disorder is dry age-related macular degeneration (dry AMD) (e.g., dry AMD with geographic atrophy), multiple sclerosis (MS), peripheral neuropathies (e.g., Guillain Barr syndrome (GBS), Chronic inflammatory demyelinating polyneuropathy (CIDP), etc.), neuromyelitis optica (NO), myasthenia gravis (MG), Alzheimer's disease, frontotemporal dementia (FTD), iatrogenic neuroinflammation (e.g., inflammation associated with adeno-associated virus administration, ASO administration, antibody administration (such as Amyloid-Related Imaging Abnormalities (ARIA), for example, Amyloid-Related Imaging Abnormalities edema (ARIA-E) neuroinflammation), etc.), and acute neuronal injury (e.g., traumatic brain injury (TBI), spinal cord injury, stroke, etc.).

35. The method of any one of claims 29 to 34, wherein the administration is systemic administration, optionally wherein the systemic administration comprises intravenous injection.

36. The method of any one of claims 29 to 34, wherein the administration comprises direct administration to the central nervous system (CNS), optionally wherein the direct administration comprises direct injection to the CNS.

37. The method of claim 36 wherein the administration comprises placing the subject in a Trendelenburg position during the administration.

38. A method for preventing or treating a disease or disorder associated with neuroinflammation in a subject in need thereof, the method comprising administering to the subject the isolated nucleic acid of any one of claims 1 to 17, or the composition of claim 18 to a subject in need thereof.

39. The method of claim 38, wherein the subject is a human.

40. The method of claim 38 or 39, wherein the disease or disorder is dry age-related macular degeneration (dry AMD) (e.g., dry AMD with geographic atrophy), multiple sclerosis (MS), peripheral neuropathies (e.g., Guillain Barr syndrome (GBS), Chronic inflammatory demyelinating polyneuropathy (CIDP), etc.), neuromyelitis optica (NO), myasthenia gravis (MG), Alzheimer's disease, frontotemporal dementia (FTD), iatrogenic neuroinflammation (e.g., inflammation associated with adeno-associated virus administration, ASO administration, antibody administration (such as Amyloid-Related Imaging Abnormalities (ARIA), for example, Amyloid-Related Imaging Abnormalities edema (ARIA-E) neuroinflammation), etc.), and acute neuronal injury (e.g., traumatic brain injury (TBI), spinal cord injury, stroke, etc.).

41. The method of any one of claims 38 to 40, wherein the neuroinflammation is iatrogenic neuroinflammation.

42. The method of claim 41, wherein the iatrogenic neuroinflammation results from administration of an AAV vector, an ASO, or an antibody to the subject.

43. The method of any one of claims 38 to 42, wherein the administration comprises direct administration to a target tissue of the subject, optionally wherein the direct administration comprises direct injection to the central nervous system (CNS), direct injection to the peripheral nervous system (PNS), or direct administration to the eye.


SEQUENCE LISTING (212 sequences)

SEQ ID NO: 1 - DNA, 18 bp, synthetic construct
  source 1..18: mol_type = other DNA
gaccaggtaggtgtagta

SEQ ID NO: 2 - DNA, 18 bp, synthetic construct
  source 1..18: mol_type = other DNA
tgaagtggatctggtagg

SEQ ID NO: 3 - DNA, 20 bp, synthetic construct
  source 1..20: mol_type = other DNA
tgtctggatgaagaggtacc

SEQ ID NO: 4 - DNA, 20 bp, synthetic construct
  source 1..20: mol_type = other DNA
tcagtggagaagacctgctg

SEQ ID NO: 5 - DNA, 20 bp, synthetic construct
  source 1..20: mol_type = other DNA
tagaatttctctgtaggctc

SEQ ID NO: 6 - DNA, 20 bp, synthetic construct
  source 1..20: mol_type = other DNA
ccggaatgtcccaagacaag

SEQ ID NO: 7 - DNA, 20 bp, synthetic construct
  source 1..20: mol_type = other DNA
aggtggcctgcacggtcacg

SEQ ID NO: 8 - DNA, 20 bp, synthetic construct
  source 1..20: mol_type = other DNA
acgttgcccatgtggttggt

SEQ ID NO: 9 - DNA, 20 bp, synthetic construct
  source 1..20: mol_type = other DNA
ctgtctggatgaagaggtac

SEQ ID NO: 10 - DNA, 20 bp, synthetic construct
  source 1..20: mol_type = other DNA
cctggcattgtttctggttc

SEQ ID NO: 11 - DNA, 20 bp, synthetic construct
  source 1..20: mol_type = other DNA
tcaatggccatgatgtactc

SEQ ID NO: 12 - DNA, 20 bp, synthetic construct
  source 1..20: mol_type = other DNA
agacctgctgtggtgagttt

SEQ ID NO: 13 - DNA, 18 bp, synthetic construct
  source 1..18: mol_type = other DNA
ggtgaagtggatctggta

SEQ ID NO: 14 - DNA, 18 bp, synthetic construct
  source 1..18: mol_type = other DNA
gagagaagaccttgacca

SEQ ID NO: 15 - DNA, 18 bp, synthetic construct
  source 1..18: mol_type = other DNA
tctgtctggatgaagagg

SEQ ID NO: 16 - DNA, 18 bp, synthetic construct
  source 1..18: mol_type = other DNA
tcttggtgaagtggatct

SEQ ID NO: 17 - DNA, 20 bp, synthetic construct
  source 1..20: mol_type = other DNA
tggtgagttttcatagtagg

SEQ ID NO: 18 - DNA, 18 bp, synthetic construct
  source 1..18: mol_type = other DNA
gaccaggtaggtgtagta

SEQ ID NO: 19 - DNA, 18 bp, synthetic construct
  source 1..18: mol_type = other DNA
ggtgtagatggtcttgtc

SEQ ID NO: 20 - DNA, 20 bp, synthetic construct
  source 1..20: mol_type = other DNA
ggctccactatgacctcgaa

SEQ ID NO: 21 - DNA, 20 bp, synthetic construct
  source 1..20: mol_type = other DNA
tctcctcgctctccagccgc

SEQ ID NO: 22 - DNA, 18 bp, synthetic construct
  source 1..18: mol_type = other DNA
gcaggagaattctggtct

SEQ ID NO: 23 - DNA, 18 bp, synthetic construct
  source 1..18: mol_type = other DNA
ctgtctggatgaagaggt

SEQ ID NO: 24 - DNA, 20 bp, synthetic construct
  source 1..20: mol_type = other DNA
ccctcacctttggctgtggt

SEQ ID NO: 25 - DNA, 20 bp, synthetic construct
  source 1..20: mol_type = other DNA
atgggactccccagagccag

SEQ ID NO: 26 - DNA, 20 bp, synthetic construct
  source 1..20: mol_type = other DNA
gtactccttcacctcaaact

SEQ ID NO: 27 - DNA, 20 bp, synthetic construct
  source 1..20: mol_type = other DNA
gaccagctgtgagtgtaggc

SEQ ID NO: 28 - DNA, 20 bp, synthetic construct
  source 1..20: mol_type = other DNA
ctctgcaggctgaccagcac

SEQ ID NO: 29 - DNA, 18 bp, synthetic construct
  source 1..18: mol_type = other DNA
gtcttggtgaagtggatc

SEQ ID NO: 30 - DNA, 20 bp, synthetic construct
  source 1..20: mol_type = other DNA
tttctctgtaggctccacta

SEQ ID NO: 31 - DNA, 18 bp, synthetic construct
  source 1..18: mol_type = other DNA
catgaggtcaaagggcat

SEQ ID NO: 32 - DNA, 20 bp, synthetic construct
  source 1..20: mol_type = other DNA
gtcttgtctgtctggatgaa

SEQ ID NO: 33 - DNA, 18 bp, synthetic construct
  source 1..18: mol_type = other DNA
gtgggtcccatggtgctg

SEQ ID NO: 34 - DNA, 20 bp, synthetic construct
  source 1..20: mol_type = other DNA
gtctgtctggatgaagaggt

SEQ ID NO: 35 - DNA, 20 bp, synthetic construct
  source 1..20: mol_type = other DNA
ggtcagcacagtcttctcac

SEQ ID NO: 36 - DNA, 20 bp, synthetic construct
  source 1..20: mol_type = other DNA
tatctctgttcattgagcca

SEQ ID NO: 37 - DNA, 20 bp, synthetic construct
  source 1..20: mol_type = other DNA
agcaccatggtctcctcgct

SEQ ID NO: 38 - DNA, 18 bp, synthetic construct
  source 1..18: mol_type = other DNA
ggtagatgatgagggtgt

SEQ ID NO: 39 - DNA, 20 bp, synthetic construct
  source 1..20: mol_type = other DNA
ctggcccatgctgtgaggag

SEQ ID NO: 40 - DNA, 18 bp, synthetic construct
  source 1..18: mol_type = other DNA
gtctggatgaagaggtac

SEQ ID NO: 41 - DNA, 18 bp, synthetic construct
  source 1..18: mol_type = other DNA
ctggatgaagaggtaccc

SEQ ID NO: 42 - DNA, 18 bp, synthetic construct
  source 1..18: mol_type = other DNA
tgtctggatgaagaggta

SEQ ID NO: 43 - DNA, 18 bp, synthetic construct
  source 1..18: mol_type = other DNA
cttcacctcaaactcagt

SEQ ID NO: 44 - DNA, 20 bp, synthetic construct
  source 1..20: mol_type = other DNA
cctgacatacttgacgagtt

SEQ ID NO: 45 - DNA, 18 bp, synthetic construct
  source 1..18: mol_type = other DNA
gtagatgatgagggtgtt

SEQ ID NO: 46 - DNA, 20 bp, synthetic construct
  source 1..20: mol_type = other DNA
tgtctgtctggatgaagagg

SEQ ID NO: 47 - DNA, 20 bp, synthetic construct
  source 1..20: mol_type = other DNA
aggtagatgatgagggtgtt

SEQ ID NO: 48 - DNA, 20 bp, synthetic construct
  source 1..20: mol_type = other DNA
tctgtaggttcatgtagttg

SEQ ID NO: 49 - DNA, 18 bp, synthetic construct
  source 1..18: mol_type = other DNA
tagatggtcttgtctgtc

SEQ ID NO: 50 - DNA, 20 bp, synthetic construct
  source 1..20: mol_type = other DNA
gagctgggttgtctgaaggc

SEQ ID NO: 51 - DNA, 18 bp, synthetic construct
  source 1..18: mol_type = other DNA
ggtcttgtctgtctggat

SEQ ID NO: 52 - DNA, 20 bp, synthetic construct
  source 1..20: mol_type = other DNA
ggcaagacgccaagctggtt

SEQ ID NO: 53 - DNA, 20 bp, synthetic construct
  source 1..20: mol_type = other DNA
gttctgagaagacaaggagt

SEQ ID NO: 54 - DNA, 18 bp, synthetic construct
  source 1..18: mol_type = other DNA
tcatgtagttggcttcaa

SEQ ID NO: 55 - DNA, 20 bp, synthetic construct
  source 1..20: mol_type = other DNA
cacctcaaactcagtggaga

SEQ ID NO: 56 - DNA, 20 bp, synthetic construct
  source 1..20: mol_type = other DNA
tgacgagttccggaatgtcc

SEQ ID NO: 57 - DNA, 20 bp, synthetic construct
  source 1..20: mol_type = other DNA
agtccctcacctggcggtga

SEQ ID NO: 58 - DNA, 20 bp, synthetic construct
  source 1..20: mol_type = other DNA
cttaccgtactccttcacct

SEQ ID NO: 59 - DNA, 20 bp, synthetic construct
  source 1..20: mol_type = other DNA
agagagaagaccttgaccac

SEQ ID NO: 60 - DNA, 18 bp, synthetic construct
  source 1..18: mol_type = other DNA
ttgtctgtctggatgaag

SEQ ID NO: 61 - DNA, 20 bp, synthetic construct
  source 1..20: mol_type = other DNA
gcattgtttctggttctctt

SEQ ID NO: 62 - DNA, 20 bp, synthetic construct
  source 1..20: mol_type = other DNA
gctcaatggccatgatgtac

SEQ ID NO: 63 - DNA, 18 bp, synthetic construct
  source 1..18: mol_type = other DNA
tagatgatgagggtgttc

SEQ ID NO: 64 - DNA, 18 bp, synthetic construct
  source 1..18: mol_type = other DNA
gtcttgtctgtctggatg

SEQ ID NO: 65 - DNA, 20 bp, synthetic construct
  source 1..20: mol_type = other DNA
ttgacggtgaagatccgata

SEQ ID NO: 66 - DNA, 20 bp, synthetic construct
  source 1..20: mol_type = other DNA
gagcttgttcagctttccat

SEQ ID NO: 67 - DNA, 20 bp, synthetic construct
  source 1..20: mol_type = other DNA
gcttggctgggtgactgtac

SEQ ID NO: 68 - DNA, 20 bp, synthetic construct
  source 1..20: mol_type = other DNA
acgaacttgttgcgcccctt

SEQ ID NO: 69 - DNA, 18 bp, synthetic construct
  source 1..18: mol_type = other DNA
gtgtagatggtcttgtct

SEQ ID NO: 70 - DNA, 20 bp, synthetic construct
  source 1..20: mol_type = other DNA
ttggtgaagtggatctggta

SEQ ID NO: 71 - DNA, 20 bp, synthetic construct
  source 1..20: mol_type = other DNA
gtcttggtgaagtggatctg

SEQ ID NO: 72 - DNA, 18 bp, synthetic construct
  source 1..18: mol_type = other DNA
ccatgaggtcaaagggca

SEQ ID NO: 73 - DNA, 20 bp, synthetic construct
  source 1..20: mol_type = other DNA
tgggcagttggagggacaca

SEQ ID NO: 74 - DNA, 18 bp, synthetic construct
  source 1..18: mol_type = other DNA
cttgtctgtctggatgaa

SEQ ID NO: 75 - DNA, 20 bp, synthetic construct
  source 1..20: mol_type = other DNA
ctccagccgcaagatgttgg

SEQ ID NO: 76 - DNA, 18 bp, synthetic construct
  source 1..18: mol_type = other DNA
ttggtgaagtggatctgg

SEQ ID NO: 77 - DNA, 18 bp, synthetic construct
  source 1..18: mol_type = other DNA
tctggatgaagaggtacc

SEQ ID NO: 78 - DNA, 20 bp, synthetic construct
  source 1..20: mol_type = other DNA
caggcccttctcgttataga

SEQ ID NO: 79 - DNA, 20 bp, synthetic construct
  source 1..20: mol_type = other DNA
caaggagtcctgcttgaccg

SEQ ID NO: 80 - DNA, 20 bp, synthetic construct
  source 1..20: mol_type = other DNA
aagtctcctgctttagtgat

SEQ ID NO: 81 - DNA, 20 bp, synthetic construct
  source 1..20: mol_type = other DNA
cgtgaaggtgacgttgccca

SEQ ID NO: 82 - DNA, 20 bp, synthetic construct
  source 1..20: mol_type = other DNA
acagtgtaggatctctgtag

SEQ ID NO: 83 - DNA, 18 bp, synthetic construct
  source 1..18: mol_type = other DNA
tgtctgtctggatgaaga

SEQ ID NO: 84 - DNA, 20 bp, synthetic construct
  source 1..20: mol_type = other DNA
acagtgtaggatctctgtag

SEQ ID NO: 85 - DNA, 20 bp, synthetic construct
  source 1..20: mol_type = other DNA
tccactatgacctcgaaact

SEQ ID NO: 86 - DNA, 20 bp, synthetic construct
  source 1..20: mol_type = other DNA
tagaatttctctgtaggctc

SEQ ID NO: 87 - DNA, 20 bp, synthetic construct
  source 1..20: mol_type = other DNA
tatctctgttcattgagcca

SEQ ID NO: 88 - DNA, 20 bp, synthetic construct
  source 1..20: mol_type = other DNA
ctcaatgttgaccatgaccg

SEQ ID NO: 89 - DNA, 20 bp, synthetic construct
  source 1..20: mol_type = other DNA
tcgttatagatgtagtagaa

SEQ ID NO: 90 - DNA, 18 bp, synthetic construct
  source 1..18: mol_type = other DNA
ggtcaccctctatcttca

SEQ ID NO: 91 - DNA, 20 bp, synthetic construct
  source 1..20: mol_type = other DNA
tggctggcacctcaatgttg

SEQ ID NO: 92 - DNA, 20 bp, synthetic construct
  source 1..20: mol_type = other DNA
cgtactccttcacctcaaac

SEQ ID NO: 93 - DNA, 20 bp, synthetic construct
  source 1..20: mol_type = other DNA
tgtctggatgaagaggtacc

SEQ ID NO: 94 - DNA, 18 bp, synthetic construct
  source 1..18: mol_type = other DNA
tggtcttgtctgtctgga

SEQ ID NO: 95 - DNA, 18 bp, synthetic construct
  source 1..18: mol_type = other DNA
gtctggatgaagaggtac

SEQ ID NO: 96 - DNA, 18 bp, synthetic construct
  source 1..18: mol_type = other DNA
gagagaagaccttgacca

SEQ ID NO: 97 - DNA, 18 bp, synthetic construct
  source 1..18: mol_type = other DNA
tgtagatggtcttgtctg

SEQ ID NO: 98 - DNA, 18 bp, synthetic construct
  source 1..18: mol_type = other DNA
gggtgtagatggtcttgt

SEQ ID NO: 99 - DNA, 20 bp, synthetic construct
  source 1..20: mol_type = other DNA
tgaccagcaccaccttctcc

SEQ ID NO: 100 - DNA, 20 bp, synthetic construct
  source 1..20: mol_type = other DNA
ctcctcttaccgtactcctt

SEQ ID NO: 101 - DNA, 18 bp, synthetic construct
  source 1..18: mol_type = other DNA
tctgtctggatgaagagg

SEQ ID NO: 102 - DNA, 18 bp, synthetic construct
  source 1..18: mol_type = other DNA
atggtcttgtctgtctgg

SEQ ID NO: 103 - DNA, 18 bp, synthetic construct
  source 1..18: mol_type = other DNA
tgtaggatctctgtaggt

SEQ ID NO: 104 - DNA, 20 bp, synthetic construct
  source 1..20: mol_type = other DNA
aagatccgatagagaactgg

SEQ ID NO: 105 - DNA, 20 bp, synthetic construct
  source 1..20: mol_type = other DNA
ttgacgagttccggaatgtc

SEQ ID NO: 106 - DNA, 20 bp, synthetic construct
  source 1..20: mol_type = other DNA
tgacctcgaaactgggcagc

SEQ ID NO: 107 - DNA, 18 bp, synthetic construct
  source 1..18: mol_type = other DNA
gaccagctgtgagtgtag

SEQ ID NO: 108 - DNA, 18 bp, synthetic construct
  source 1..18: mol_type = other DNA
ggctggattgtggagtag

SEQ ID NO: 109 - DNA, 20 bp, synthetic construct
  source 1..20: mol_type = other DNA
cagcttgtggttgacggtga

SEQ ID NO: 110 - DNA, 20 bp, synthetic construct
  source 1..20: mol_type = other DNA
ctcacctggcggtgatggtg

SEQ ID NO: 111 - DNA, 20 bp, synthetic construct
  source 1..20: mol_type = other DNA
tattgagccaaggcttggaa

SEQ ID NO: 112 - DNA, 18 bp, synthetic construct
  source 1..18: mol_type = other DNA
ggaccaggtaggtgtagt

SEQ ID NO: 113 - DNA, 18 bp, synthetic construct
  source 1..18: mol_type = other DNA
tcttgtctgtctggatga

SEQ ID NO: 114 - DNA, 20 bp, synthetic construct
  source 1..20: mol_type = other DNA
ggtgtagatggtcttgtctg

SEQ ID NO: 115 - DNA, 20 bp, synthetic construct
  source 1..20: mol_type = other DNA
ttgaactccctgttggctgg

SEQ ID NO: 116 - DNA, 20 bp, synthetic construct
  source 1..20: mol_type = other DNA
accatgaccgtccggcccac

SEQ ID NO: 117 - DNA, 16 bp, synthetic construct
  source 1..16: mol_type = other DNA
gaggagtgagcagcgc

SEQ ID NO: 118 - DNA, 20 bp, synthetic construct
  source 1..20: mol_type = other DNA
caagctggttctgagaagac

SEQ ID NO: 119 - DNA, 18 bp, synthetic construct
  source 1..18: mol_type = other DNA
ccacgggtagcagcttgt

SEQ ID NO: 120 - DNA, 20 bp, synthetic construct
  source 1..20: mol_type = other DNA
ggcacctcaatgttgaccat

SEQ ID NO: 121 - DNA, 18 bp, synthetic construct
  source 1..18: mol_type = other DNA
ggctctatgagaaagagg

SEQ ID NO: 122 - DNA, 20 bp, synthetic construct
  source 1..20: mol_type = other DNA
tgtctggatgaagaggtacc

SEQ ID NO: 123 - DNA, 20 bp, synthetic construct
  source 1..20: mol_type = other DNA
gatagagtacctgtcggagt

SEQ ID NO: 124 - DNA, 20 bp, synthetic construct
  source 1..20: mol_type = other DNA
cagctgtgactgtgaaaccc

SEQ ID NO: 125 - DNA, 20 bp, synthetic construct
  source 1..20: mol_type = other DNA
gtgatgatagagtacctgtc

SEQ ID NO: 126 - DNA, 20 bp, synthetic construct
  source 1..20: mol_type = other DNA
gttggctgggatctaggcgt

SEQ ID NO: 127 - DNA, 18 bp, synthetic construct
  source 1..18: mol_type = other DNA
tctggatgaagaggtacc

SEQ ID NO: 128 - DNA, 20 bp, synthetic construct
  source 1..20: mol_type = other DNA
gcttgaccgggatgccttcc

SEQ ID NO: 129 - DNA, 20 bp, synthetic construct
  source 1..20: mol_type = other DNA
gaagatgacaaaggcagttc

SEQ ID NO: 130 - DNA, 18 bp, synthetic construct
  source 1..18: mol_type = other DNA
gtctgtctggatgaagag

SEQ ID NO: 131 - DNA, 20 bp, synthetic construct
  source 1..20: mol_type = other DNA
ccaagacaagggcaagacgc

SEQ ID NO: 132 - DNA, 18 bp, synthetic construct
  source 1..18: mol_type = other DNA
aggtagatgatgagggtg

SEQ ID NO: 133 - DNA, 20 bp, synthetic construct
  source 1..20: mol_type = other DNA
tcaatggccatgatgtactc

SEQ ID NO: 134 - DNA, 20 bp, synthetic construct
  source 1..20: mol_type = other DNA
ctgcactcaccgtgaaggtg

SEQ ID NO: 135 - DNA, 18 bp, synthetic construct
  source 1..18: mol_type = other DNA
gtagatggtcttgtctgt

SEQ ID NO: 136 - DNA, 18 bp, synthetic construct
  source 1..18: mol_type = other DNA
gtctgtctggatgaagag

SEQ ID NO: 137 - DNA, 18 bp, synthetic construct
  source 1..18: mol_type = other DNA
gtcttggtgaagtggatc

SEQ ID NO: 138 - DNA, 20 bp, synthetic construct
  source 1..20: mol_type = other DNA
cttggtgaagtggatctggt

SEQ ID NO: 139 - DNA, 20 bp, synthetic construct
  source 1..20: mol_type = other DNA
tctgtaggttcatgtagttg

SEQ ID NO: 140 - DNA, 20 bp, synthetic construct
  source 1..20: mol_type = other DNA
aggcacaaagtcaaagtctt

SEQ ID NO: 141 - DNA, 20 bp, synthetic construct
  source 1..20: mol_type = other DNA
ctcacatgggactccccaga

SEQ ID NO: 142 - DNA, 20 bp, synthetic construct
  source 1..20: mol_type = other DNA
ctgagaagacaaggagtcct

SEQ ID NO: 143 - DNA, 18 bp, synthetic construct
  source 1..18: mol_type = other DNA
tcttggtgaagtggatct

SEQ ID NO: 144 - DNA, 18 bp, synthetic construct
  source 1..18: mol_type = other DNA
gtagcagcttgtggttga

SEQ ID NO: 145 - DNA, 20 bp, synthetic construct
  source 1..20: mol_type = other DNA
tgtagtagaatttctctgta

SEQ ID NO: 146 - DNA, 18 bp, synthetic construct
  source 1..18: mol_type = other DNA
ggtgaagtggatctggta

SEQ ID NO: 147 - DNA, 20 bp, synthetic construct
  source 1..20: mol_type = other DNA
cttttctgacttgaactccc

SEQ ID NO: 148 - DNA, 20 bp, synthetic construct
  source 1..20: mol_type = other DNA
gtgtagatggtcttgtctgt

SEQ ID NO: 149 - DNA, 20 bp, synthetic construct
  source 1..20: mol_type = other DNA
gggacccaccttgagctctt

SEQ ID NO: 150 - DNA, 20 bp, synthetic construct
  source 1..20: mol_type = other DNA
catacttgacgagttccgga

SEQ ID NO: 151 - DNA, 20 bp, synthetic construct
  source 1..20: mol_type = other DNA
aaggagtcctgcttgaccgg

SEQ ID NO: 152 - DNA, 20 bp, synthetic construct
  source 1..20: mol_type = other DNA
ctcggatcttccactggccc

SEQ ID NO: 153 - DNA, 18 bp, synthetic construct
  source 1..18: mol_type = other DNA
ttatagatgtagtagaat

SEQ ID NO: 154 - DNA, 20 bp, synthetic construct
  source 1..20: mol_type = other DNA
gcattgtttctggttctctt

SEQ ID NO: 155 - DNA, 18 bp, synthetic construct
  source 1..18: mol_type = other DNA
agtagaatttctctgtag

SEQ ID NO: 156 - DNA, 20 bp, synthetic construct
  source 1..20: mol_type = other DNA
gtctggatgaagaggtaccc

SEQ ID NO: 157 - DNA, 20 bp, synthetic construct
  source 1..20: mol_type = other DNA
gtcttctcactggacagcac

SEQ ID NO: 158 - DNA, 20 bp, synthetic construct
  source 1..20: mol_type = other DNA
gcagaaagaagggcattggg

SEQ ID NO: 159 - DNA, 20 bp, synthetic construct
  source 1..20: mol_type = other DNA
tcatagtaggctcggatctt

SEQ ID NO: 160 - DNA, 20 bp, synthetic construct
  source 1..20: mol_type = other DNA
acagtgtaggatctctgtag

SEQ ID NO: 161 - DNA, 18 bp, synthetic construct
  source 1..18: mol_type = other DNA
ctggatgaagaggtaccc

SEQ ID NO: 162 - DNA, 20 bp, synthetic construct
  source 1..20: mol_type = other DNA
tagatggtcttgtctgtctg

SEQ ID NO: 163 - DNA, 18 bp, synthetic construct
  source 1..18: mol_type = other DNA
cttggtgaagtggatctg

SEQ ID NO: 164 - DNA, 20 bp, synthetic construct
  source 1..20: mol_type = other DNA
accatgaggtcaaagggcat

SEQ ID NO: 165 - DNA, 20 bp, synthetic construct
  source 1..20: mol_type = other DNA
atgccttccgggttctgtgg

SEQ ID NO: 166 - DNA, 20 bp, synthetic construct
  source 1..20: mol_type = other DNA
tcttggtgaagtggatctgg

SEQ ID NO: 167 - DNA, 20 bp, synthetic construct
  source 1..20: mol_type = other DNA
tggtgacctccaggcccttc

SEQ ID NO: 168 - DNA, 20 bp, synthetic construct
  source 1..20: mol_type = other DNA
gctcaagggcttctggctgg

SEQ ID NO: 169 - DNA, 20 bp, synthetic construct
  source 1..20: mol_type = other DNA
ctggcggtgatggtgacctc

SEQ ID NO: 170 - DNA, 20 bp, synthetic construct
  source 1..20: mol_type = other DNA
tctgtctggatgaagaggta

SEQ ID NO: 171 - DNA, 20 bp, synthetic construct
  source 1..20: mol_type = other DNA
gaagatgacaaaggcagttc

SEQ ID NO: 172 - DNA, 20 bp, synthetic construct
  source 1..20: mol_type = other DNA
gaggtacccgctctgcaggc

SEQ ID NO: 173 - DNA, 20 bp, synthetic construct
  source 1..20: mol_type = other DNA
tctggatgaagaggtacccg

SEQ ID NO: 174 - DNA, 20 bp, synthetic construct
  source 1..20: mol_type = other DNA
ctcaccgtgaaggtgacgtt

SEQ ID NO: 175 - DNA, 20 bp, synthetic construct
  source 1..20: mol_type = other DNA
atgaaggtggcctagaaccc

SEQ ID NO: 176 - DNA, 20 bp, synthetic construct
  source 1..20: mol_type = other DNA
ccagcctcacctgtggagcc

SEQ ID NO: 177 - DNA, 18 bp, synthetic construct
  source 1..18: mol_type = other DNA
tggtgaagtggatctggt

SEQ ID NO: 178 - DNA, 18 bp, synthetic construct
  source 1..18: mol_type = other DNA
ttggtgaagtggatctgg

SEQ ID NO: 179 - DNA, 20 bp, synthetic construct
  source 1..20: mol_type = other DNA
aaccactcacatgggactcc

SEQ ID NO: 180 - DNA, 18 bp, synthetic construct
  source 1..18: mol_type = other DNA
tggtgaagtggatctggt

SEQ ID NO: 181 - DNA, 20 bp, synthetic construct
  source 1..20: mol_type = other DNA
gattcaggcagggaaatcct

SEQ ID NO: 182 - DNA, 18 bp, synthetic construct
  source 1..18: mol_type = other DNA
taggttcatgtagttggc

SEQ ID NO: 183 - DNA, 18 bp, synthetic construct
  source 1..18: mol_type = other DNA
tggatgaagaggtacccg

SEQ ID NO: 184 - DNA, 20 bp, synthetic construct
  source 1..20: mol_type = other DNA
gtcccgaaggtggcctgcac

SEQ ID NO: 185 - DNA, 20 bp, synthetic construct
  source 1..20: mol_type = other DNA
tctcctcgctctccagccgc

SEQ ID NO: 186 - DNA, 20 bp, synthetic construct
  source 1..20: mol_type = other DNA
ggaactcaccctcagcacag

SEQ ID NO: 187 - DNA, 20 bp, synthetic construct
  source 1..20: mol_type = other DNA
ttcccgatgatgtagctgag

SEQ ID NO: 188 - DNA, 20 bp, synthetic construct
  source 1..20: mol_type = other DNA
tcgaaactgggcagcactgg

SEQ ID NO: 189 - DNA, 20 bp, synthetic construct
  source 1..20: mol_type = other DNA
tggtcttgtctgtctggatg

SEQ ID NO: 190 - DNA, 20 bp, synthetic construct
  source 1..20: mol_type = other DNA
gttctgagaagacaaggagt

SEQ ID NO: 191 - DNA, 18 bp, synthetic construct
  source 1..18: mol_type = other DNA
cttggtgaagtggatctg

SEQ ID NO: 192 - DNA, 20 bp, synthetic construct
  source 1..20: mol_type = other DNA
gtgggaacaggtgaggtttc

SEQ ID NO: 193 - DNA, 20 bp, synthetic construct
  source 1..20: mol_type = other DNA
cccacatgaggtagtgtttc

SEQ ID NO: 194 - DNA, 20 bp, synthetic construct
  source 1..20: mol_type = other DNA
ttgtatgaagcaattctctg

SEQ ID NO: 195 - DNA, 20 bp, synthetic construct
  source 1..20: mol_type = other DNA
gctggataagctctacatta

SEQ ID NO: 196 - DNA, 20 bp, synthetic construct
  source 1..20: mol_type = other DNA
gccagtcatcatggatatgt

SEQ ID NO: 197 - DNA, 20 bp, synthetic construct
  source 1..20: mol_type = other DNA
gatcatagtgttcttggcat

SEQ ID NO: 198 - DNA, 20 bp, synthetic construct
  source 1..20: mol_type = other DNA
gccttagcatggtacattgt

SEQ ID NO: 199 - DNA, 20 bp, synthetic construct
  source 1..20: mol_type = other DNA
gtgagggccatgtctttctc

SEQ ID NO: 200 - DNA, 20 bp, synthetic construct
  source 1..20: mol_type = other DNA
ccacccttcttgatgagctc

SEQ ID NO: 201 - DNA, 20 bp, synthetic construct
  source 1..20: mol_type = other DNA
gagaattctggtctcagact

SEQ ID NO: 202 - DNA, 20 bp, synthetic construct
  source 1..20: mol_type = other DNA
gctggattgtggagtagttc

SEQ ID NO: 203 - DNA, 20 bp, synthetic construct
  source 1..20: mol_type = other DNA
gtggtgatggagtctttcaa

SEQ ID NO: 204 - DNA, 20 bp, synthetic construct
  source 1..20: mol_type = other DNA
gtggtcaccctctatcttca

SEQ ID NO: 205 - DNA, 20 bp, synthetic construct
  source 1..20: mol_type = other DNA
gaccaggtaggtgtagtagc

SEQ ID NO: 206 - DNA, 20 bp, synthetic construct
  source 1..20: mol_type = other DNA
cacctcaaactcagtggaga

SEQ ID NO: 207 - DNA, 20 bp, synthetic construct
  source 1..20: mol_type = other DNA
ggtagcagcttgtggttgac

SEQ ID NO: 208 - DNA, 20 bp, synthetic construct
  source 1..20: mol_type = other DNA
gtcttctcactggacagcac

SEQ ID NO: 209 - DNA, 20 bp, synthetic construct
  source 1..20: mol_type = other DNA
cggaccaggtaggtgtagta

SEQ ID NO: 210 - DNA, 18 bp, synthetic construct
  source 1..18: mol_type = other DNA
atataactgaagctttat

SEQ ID NO: 211 - DNA, 5231 bp, Homo sapiens
  source 1..5231: mol_type = genomic DNA
actcctccccatcctctccctctgtccctctgtccctctgaccctgcactgtcccagcaccatgggacccacctcaggtcccagcctgctgctcctgctactaacccacctccccctggctctggggagtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaacccccgagcagaagacctggtggggaagtctttgtacgtgtctgccaccgtcatcttgcactcaggcagtgacatggtgcaggcagagcgcagcgggatccccatcgtgacctctccctaccagatccacttcaccaagacacccaagtacttcaaaccaggaatgccctttgacctcatggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctgatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcgctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaagatctgggacgtggtggagaaggcagacatcggctgcaccccgggcagtgggaaggattacgccggtgtcttctccgacgcagggctgaccttcacgagcagcagtggccagcagaccgcccagagggcagaacttcagtgcccgcagccagccgcccgccgacgccgttccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccaggagtaacctggatgaggacatcattgcagaagagaacatcgtttcccgaagtgagttcccagagagctggctgtggaacgttgaggacttgaaagagccaccgaaaaatggaatctctacgaagctcatgaatatatttttgaaagactccatcaccacgtgggagattctggctgtgagcatgtcggacaagaaagggatctgtgtggcagaccccttcgaggtcacagtaatgcaggacttcttcatcgacctgcggctaccctactctgttgttcgaaacgagcaggtggaaatccgagccgttctctacaattaccggcagaaccaagagctcaaggtgagggtggaactactccacaatccagccttctgcagcctggccaccaccaagaggcgtcaccagcagaccgtaaccatcccccccaagtcctcgttgtccgttccatatgtcatcgtgccgctaaagaccggcctgcaggaagtggaagtcaaggctgctgtctaccatcatttcatcagtgacggtgtcaggaagtccctgaaggtcgtgccggaaggaatcagaatgaacaaaactgtggctgttcgcaccctggatccagaacgcctgggccgtgaaggagtgcagaaagaggacatcccacctgcagacctcagtgaccaagtcccggacaccgagtctgagaccagaattctcctgcaagggaccccagtggcccagatgacagaggatgccgtcgacgcggaacggctgaagcacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagagaccaaggaaaatgagggtttcacagtcacagctgaaggaaaaggccaaggcaccttgtcggtggtgacaatgtaccatgctaaggccaaagatcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatattggacatatccatgatgactggctttgctccagacacagatgacctgaagcagctggccaatggtgttgacagatacatctccaagtatgagctggacaaagccttctccgataggaacaccctcatcatctacctggacaaggtctcacactctgaggatgactgtctagctttcaaagttcaccaatactttaatgtagagcttatccagcctggagcagtcaaggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcccgaggaggacgaatgccaagacgaagagaaccagaaacaatgccaggacctcggcgccttcaccgagagcatggttgtctttgggtgccccaactgaccacacccccattcccccactccagataaagcttcagttatatctcacgtgtctggagttctttgccaagagggagaggctgaaatccccagccgcctcacctgcagctcagctccatcctacttgaaacctcacctgttcccaccgcattttctcctggcgttcgcctgctagtgtg

SEQ ID NO: 212 - DNA, 1000 bp, Homo sapiens
  source 1..1000: mol_type = genomic DNA
actcctccccatcctctccctctgtccctctgtccctctgaccctgcactgtcccagcaccatgggacccacctcaggtcccagcctgctgctcctgctactaacccacctccccctggctctggggagtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaac